(19) United States
(12) Patent Application Publication
(10) Pub. No.: US 2023/0152316 A1
(43) Pub. Date: May 18, 2023

(54) SARS-COV2 SPIKE PROTEIN BINDING PEPTIDES

(71) Applicant: Massachusetts Institute of Technology, Cambridge, MA (US)

(72) Inventors: Bradley L. Pentelute, Cambridge, MA (US); Genwei Zhang, Cambridge, MA (US); Sebastian Pomplun, Cambridge, MA (US); Alexander R. Loftis, Cambridge, MA (US); Andrei Loas, Cambridge, MA (US)

(21) Appl. No.: 17/906,400

(22) PCT Filed: Mar. 19, 2021
(86) PCT No.: PCT/US21/23262

§ 371 Date: Sep. 15, 2022

Related U.S. Application Data
(60) Provisional application No. 62/991,886, filed on Mar. 19, 2020.
(60) Provisional application No. 63/016,925, filed on Apr. 28, 2020.

(51) Int. Cl. G01N 33/569

(52) U.S. Cl. CPC G01N 33/56983; G01N 2333/165

(57) ABSTRACT

The disclosure provides peptides that are useful for the prevention of infection of coronaviruses, especially SARS-CoV-2. Also described is a method reducing probability of viral infection in a human subject, comprising administering a therapeutically effective amount of the pharmaceutical composition described herein.

### CROSS-REFERENCE TO RELATED APPLICATIONS

This application claims the benefit of U.S. Provisional Application Ser. No. 62/991,886, filed Mar. 19, 2020, and U.S. Provisional Application Ser. No. 63/016,925, filed Apr. 28, 2020, the entire disclosures of which are incorporated herein by reference.

### SEQUENCE LISTING

The instant application contains a Sequence Listing which has been submitted electronically in ASCII format and is hereby incorporated by reference in its entirety. Said ASCII copy, created on Mar. 19, 2021, is named 713414_083474-013PC_SL.txt and is 68,614 bytes in size.

### BACKGROUND

Coronavirus disease 19 (COVID-19) is an emerging global health crisis with over 121 million cases to date. As the COVID-19 pandemic continues to expand, intense efforts from both academia and industry are focused on the development of vaccines or treatments to ameliorate symptoms and eventually, stop the virus transmission. At the present time, there are few effective treatments available in the clinic.

Many studies are underway to understand how COVID-19 infection occurs in an individual and also how it spreads throughout a population. It has been found that the spike protein of coronaviruses first binds to a host cell's surface receptor (ACE2 receptor) for viral attachment, then the virus enters into endosomes, and eventually the viral membrane fuses with the cell's membrane so that the viral RNA is released into the nucleus of the cell, where replication occurs and more viral particles are produced.

Recently, a cryo-EM structure revealed that the binding of SARS-CoV-2 spike protein (S) receptor binding domain (RBD) and the human ACE2 receptor occurs over an extended interface, such that the loop regions of the RBD span the arch-shaped at helix of the ACE2 extracellular peptidase domain (ACE2-PD) and extend to the α2 helix and the loop that connects the β3 and β4 strands. This extended binding interface between the viral spike protein RBD and the human ACE2-PD domain suggests challenges in developing small molecule inhibitors to block this protein-protein interaction leading to a therapeutic antiviral effect, which the present disclosure addresses.

### SUMMARY

This disclosure provides a composition comprising one or more peptides that specifically bind to a spike protein of a coronavirus. In some embodiments, one or more peptides specifically bind to a receptor binding domain (RBD) of the spike protein. In some embodiments, the RBD specifically binds to human ACE2 receptor. In some embodiments, the coronavirus is selected from the group consisting of human coronavirus 229E (HCoV-229E), human coronavirus OC43 (HCoV-OC43), severe acute respiratory syndrome coronavirus (SARS-CoV), human coronavirus NL63 (HCoV-NL63), human coronavirus HKU1, Middle East respiratory syndrome-related coronavirus (MERS-CoV) and severe acute respiratory syndrome coronavirus 2 (SARS-CoV-2).

In some embodiments, the coronavirus is SARS-CoV-2.

In some embodiments, the one or more peptides are 6-100, 6-70 or 6-25 amino acids in length. In some embodiments, the one or more peptides comprise an amino acid sequence at least 60% identical to a sequence selected from the group consisting of IEEQAKTFLDKFNHEAEDLFYQS (SEQ ID NO: 3), TFLDKFNHEAED (SEQ ID NO: 15), IEEQAKTFLDKFNHEAE (SEQ ID NO: 40), TFLDKFNHEAEDLFYQS (SEQ ID NO: 14), and TCLDKCNH (SEQ ID NO: 41) or derivatives thereof. In some embodiments, the one or more peptides comprise an amino acid sequence selected from the group consisting of IEEQAKTFLDKFNHEAEDLFYQS (SEQ ID NO: 3), TFLDKFNHEAED (SEQ ID NO: 15), IEEQAKTFLDKFNHEAE (SEQ ID NO: 40), TFLDKFNHEAEDLFYQS (SEQ ID NO: 14), and TCLDKCNH (SEQ ID NO: 41) or derivatives thereof.

In some embodiments, the one or more peptides comprise the formula: X₁X₂X₃X₄X₅X₆X₇X₈LDX₁₁X₁₂NHEX₁₆EDX₁₉X₂₀X₂₁X₂₂X₂₃ (SEQ ID NO: 133); wherein: X₁ is I or absent; X₂ is E or absent; X₃ is E or absent; X₄ is Q or absent; X₅ is A, Aib, absent, or X₅ taken together with X₈ or X₁₂ is represented by Formula (I) below:

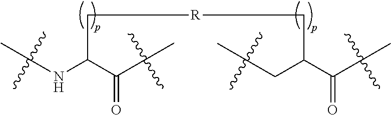

X₆ is K, K*, A, S, Q, H, T, or absent; X₇ is T or Aib; X₈ is F, S, T, H, N, D, or X₈ taken together with X₅, X₁₂, or X₁₆ is represented by Formula (I); X₁₁ is K or Q; X₁₂ is F, Aib, or X₁₂ taken together with X₅, X₈, or X₁₆ is represented by Formula (I); X₁₆ is A, Aib, or X₁₆ taken together with X₈, X₁₂, or X₂₀ is represented by Formula (I); X₁₉ is L or absent; X₂₀ is F, Aib, absent, or X₂₀ taken together with X₁₆ is represented by Formula (I); X₂₁ is Y or absent; X₂₂ is Q or absent; X₂₃ is S or absent; wherein when X₂, X₃, X₄, X₅, or X₆ are absent, all previous residues are also absent; wherein when X₁₉, X₂₀, X₂₁, or X₂₂ are absent, all subsequent residues are also absent; wherein K* represents a lysine residue covalently bound at its ζ-nitrogen with C(O)(CH2)mCH₃ or the formula below:

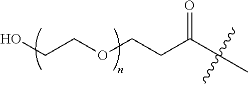

wherein the N-terminal amino group of the peptide is optionally substituted with C(O)(CH₂)ₘCH₃ or the formula below:

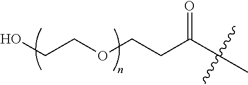

wherein the C-terminus of the peptide comprises a C-terminal acid group or a C-terminal amide group; wherein R is —NHC(O)—, —S—S—, —S-(aryl)-S—, —S-(aryl)-(aryl)-S—, —S-(perfluoroaryl)-S—, —S-(perfluoroaryl)-(perfluoroaryl)-S— —S—(C₁₋₆ alkyl)-S—, —S—CH₂—C(O)—CH₂—S—, —NH-(aryl)-NH—, —NHC(O)-(aryl)-C(O)NH—, —NHC(O)-(aryl)-(aryl)-C(O)NH—, —NHC(O)-(perfluoroaryl)-C(O)NH—, —NHC(O)-(perfluoroaryl)-(perfluoroaryl)-C(O)NH—, —NHC(O)—(C₁₋₆ alkyl)-C(O)NH—, —NHC(O)—CH₂—C(O)—CH₂—C(O)NH—, C₂₋₆ alkenyl, or heteroaryl; m is for each occurrence independently 0-16; n is for each occurrence independently 4-1000; and p is for each occurrence independently 1-6. In some embodiments, m is 0. In some other embodiments, m is 14.

In some embodiments, the formula below:

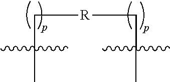

is selected from the group consisting of the formulas below:

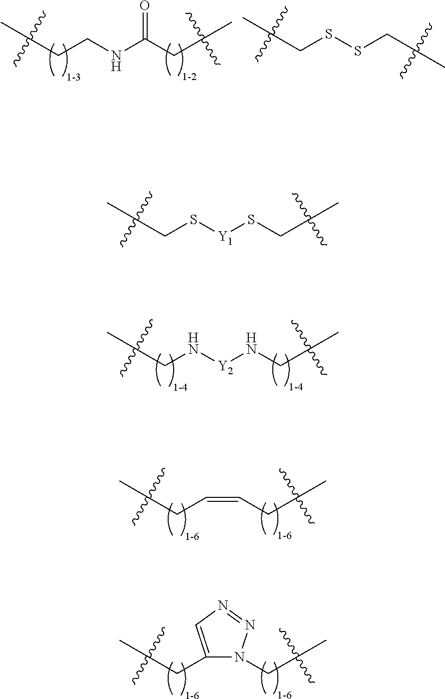

and wherein: Y1 is aryl, -aryl-aryl-, perfluoroaryl, -perfluoroaryl-perfluoroaryl-, C₁₋₆ alkyl, or —CH2-C(O)—CH2-; and Y2 is aryl or —C(O)—Y1-C(O)—.

In some embodiments, formula below:

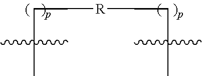

is

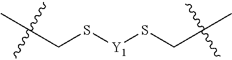

and Y1 is perfluoroaryl or -perfluoroaryl-perfluoroaryl-.
In some embodiments, Y1 is the formula below:

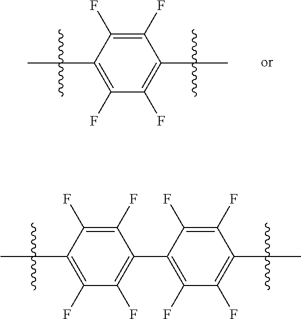 [SMILES: *c1c(F)c(F)c(*)c(F)c1F ; *c1c(F)c(F)c(-c2c(F)c(F)c(*)c(F)c2F)c(F)c1F]

In some embodiments, the one or more peptides comprise an amino acid sequence selected from the group consisting of: IEEQAKTFLDKFNHEAEDLFYQS (SEQ ID NO: 3), IEEQAKTFLDKFNHEAEDLFYQ (SEQ ID NO: 4), IEEQAKTFLDKFNHEAEDLFY (SEQ ID NO: 5), IEEQAKTFLDKFNHEAEDLF (SEQ ID NO: 6), IEEQAKTFLDKFNHEAEDL (SEQ ID NO: 7), IEEQAKTFLDKFNHEAED (SEQ ID NO: 8), EEQAKTFLDKFNHEAEDLFYQS (SEQ ID NO: 9), EQAKTFLDKFNHEAEDLFYQS (SEQ ID NO: 10), QAKTFLDKFNHEAEDLFYQS (SEQ ID NO: 11), AKTFLDKFNHEAEDLFYQS (SEQ ID NO: 12), KTFLDKFNHEAEDLFYQS (SEQ ID NO: 13), TFLDKFNHEAEDLFYQS (SEQ ID NO: 14), IEEQAK*TFLDKFNHEAEDLFYQS (SEQ ID NO: 17), IEEQAATFLDKFNHEAEDLFYQS (SEQ ID NO: 18), IEEQASTFLDKFNHEAEDLFYQS (SEQ ID NO: 19), IEEQAQTFLDKFNHEAEDLFYQS (SEQ ID NO: 20), IEEQAHTFLDKFNHEAEDLFYQS (SEQ ID NO: 21), IEEQATTFLDKFNHEAEDLFYQS (SEQ ID NO: 22), IEEQAKTSLDKFNHEAEDLFYQS (SEQ ID NO: 23), IEEQAKTTLDKFNHEAEDLFYQS (SEQ ID NO: 24), IEEQAKTHLDKFNHEAEDLFYQS (SEQ ID NO: 25), IEEQAKTNLDKFNHEAEDLFYQS (SEQ ID NO: 26), IEEQAKTDLDKFNHEAEDLFYQS (SEQ ID NO: 27), IEEQAKTFLDQFNHEAEDLFYQS (SEQ ID NO: 28), IEEQAibKTFLDKFNHEAEDLFYQS (SEQ ID NO: 29), IEEQAKAibFLDKFNHEAEDLFYQS (SEQ ID NO: 30), IEEQAKTFLDKAibNHEAEDLFYQS (SEQ ID NO: 31), IEEQAKTFLDKFNHEAibEDLFYQS (SEQ ID NO: 32), IEEQAKTFLDKFNHEAEDLAibYQS (SEQ ID NO: 33), STIEEQAKTFLDKFNHEAEDLFYQSSLASWNYNTNITEENVQNMNNAGDKWSAFLK EQSTLAQMYPLQEIQ (SEQ ID NO: 16), and STIEEQAKTFLDKFNHEAEDLFYQSSLASWNYNTNITEENVQNMNNAGDKWSAFLK EQSTLAQMYPLQEIQNLTVKLQLQALQQN (SEQ ID NO: 42).

In some embodiments, one of the one or more peptides comprises the amino acid sequence of IEEQAKTFLDKFNHEAEDLFYQS (SEQ IN NO 3).

In some embodiments, X₅ and X₈ are taken together to form a group represented by Formula (I); X₅ and X₁₂ are taken together to form a group represented by Formula (I); X₈ and X₁₂ are taken together to form a group represented by Formula (I); X₈ and X₁₆ are taken together to form a group represented by Formula (I), X₁₂ and X₁₆ are taken together to form a group represented by Formula (I); or X₁₆ and X₂₀ are taken together to form a group represented by Formula (I). In some embodiments, the one or more peptides comprise the formula:

(SEQ ID NO: 134)
X₀X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂
X₂₃X₂₄;

wherein:
X₀ is QST, AQMYPLQEG (SEQ ID NO: 135), AQMYPLQEGG (SEQ ID NO: 136), or absent;
X₁ is I or absent;
X₂ is E or absent;
X₃ is E or absent;
X₄ is Q or absent;
X₅ is A, K, or 2-amino isobutyric acid (A/b), absent, or X₅ taken together with X₈ or X₁₂ is represented by Formula (I):

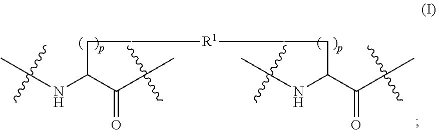

X₆ is K, K*, A, S, Q, H, T, or absent;
X₇ is T, L, D, Y, A, Aib, absent or is represented by Formula (II):

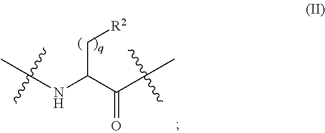

X₈ is F, S, T, H, N, D, E, A, Aib, absent, or X₈ taken together with X₅, X₁₂, or X₁₆ is represented by Formula (I);
X₉ is L, E, A, or absent;
X₁₀ is D, E, A, cysteic acid (Cya), or absent;
X₁₁ is K, Q, W, A, absent, or is represented by Formula (II):
X₁₂ is F, K, A. Aib, absent, or X₁₂ taken together with X₅, X₈, or X₁₆ is represented by Formula (I);
X₁₃ is N, K, H, D, A, or absent;
X₁₄ is H, E, V, A, Aib, 4-aminopiperidine-4-carboxylic acid (Pip), 4-fluorophenylalanine (Ff), or absent;
X₁₅ is E, A, or absent;
X₁₆ is A, E, Aib, Pip, absent, or X₁₆ taken together with X₈, X₁₂, or X₂₀ is represented by Formula (I);
X₁₇ is E, A, or absent;
X₁₈ is D, A, or absent;
X₁₉ is L, K, A, Aib, omithine (Orn), Pip or absent;
X₂₀ is F, K, A, Aib, absent, or X₂₀ taken together with X₁₆ is represented by Formula (I);
X₂₁ is Y, A, absent, or is represented by Formula (II);
X₂₂ is Q, K, or absent;
X₂₃ is S, Aib or absent;
X₂₄ is S, SLAS (SEQ ID NO: 137), SGGLGKGDFR (SEQ ID NO: 138), SpLGKGDFR (SEQ ID NO: 139), SkLGKGDFR (SEQ ID NO: 140), SGLGKGDFR (SEQ ID NO: 141), SGLGKGCyaFR (SEQ ID NO: 142), or absent;

wherein when any of X₁ through X₁₂ are absent, all residues prior to the absent residue are also absent;

wherein when any of X₁₃ through X₂₃ are absent, all residues subsequent to the absent residue are also absent;

wherein at least 12 consecutive residues are not absent;

wherein K* represents a lysine residue covalently bound at its ζ-nitrogen with C(O)(CH₂)ₘCH₃;

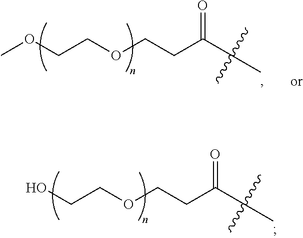

wherein the N-terminal amino group of the peptide is optionally substituted with C(O)(CH₂)ₘCH₃,

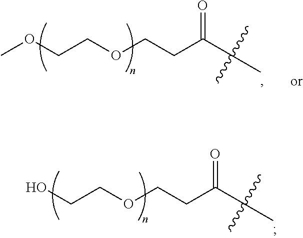

wherein the C-terminus of the peptide comprises a C-terminal acid group or a C-terminal amide group;

wherein R¹ is —NHC(O)—, —S—S—, —S-(aryl)-S—, —S-(aryl)-(aryl)-S—, —S-(perfluoroaryl)-S—, —S-(perfluoroaryl)-(perfluoroaryl)-S— —S—(C₁₋₆ alkyl)-S—, —S—CH₂—C(O)—CH₂—S—, —NH-(aryl)-NH—, —NHC(O)-(aryl)-C(O)NH—, —NHC(O)-(aryl)-(aryl)-C(O)NH—, —NHC(O)-(perfluoroaryl)-C(O)NH—, —NHC(O)-(perfluoroaryl)-(perfluoroaryl)-C(O)NH—, —NHC(O)—(C₁₋₆ alkyl)-C(O)NH—, —NHC(O)—CH₂—C(O)—CH₂—C(O)NH—, C₂₋₆ alkenyl, or heteroaryl;

wherein R² is aryl, heteroaryl, or C₃₋₈ cycloalkyl, each of which is optionally substituted with 1, 2, or substituents independently selected from the group consisting of C₁₋₆ alkyl, aryl, nitro, halo, cyano, amino, hydroxy, and C₁₋₃ alkylamino;
m is for each occurrence independently 0-16;
n is for each occurrence independently 4-1000; and
p is for each occurrence independently 1-6;
q is 1-3.

In some embodiments,
X₇ is T, L, D, Y, A, and Aib;
X₈ is F, S, T, H, N, D, E, A, Aib, or X₈ taken together with X₅, X₁₂, or X₁₆ is represented by Formula (I);
X₉ is L, E, or A;
X₁₀ is D, E, A, or Cya;
X₁₁ is K, Q, W, A, or is represented by Formula (II):

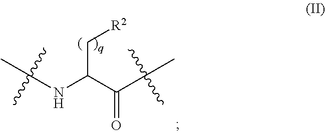

X₁₂ is F, K, A, Aib, or X₁₂ taken together with X₅, X₈, or X₁₆ is represented by Formula (I);
X₁₃ is N, K, H, D, or A;
X₁₄ is H, E, V, A, Aib, Pip, or Ff;
X₁₅ is E or A;
X₁₆ is A, E, Aib, Pip, or X₁₆ taken together with X₈, X₁₂, or X₂₀ is represented by Formula (I);
X₁₇ is E or A;
X₁₈ is D or A;

wherein when X₁, X₂, X₃, X₄, X₅, or X₆ are absent, all previous residues are also absent; and

wherein when X₁₉, X₂₀, X₂₁, X₂₂, or X₂₃ are absent, all subsequent residues are also absent;

In some embodiments, at least 15 consecutive residues are not absent.

In some embodiments, R₂ is selected from the group consisting of

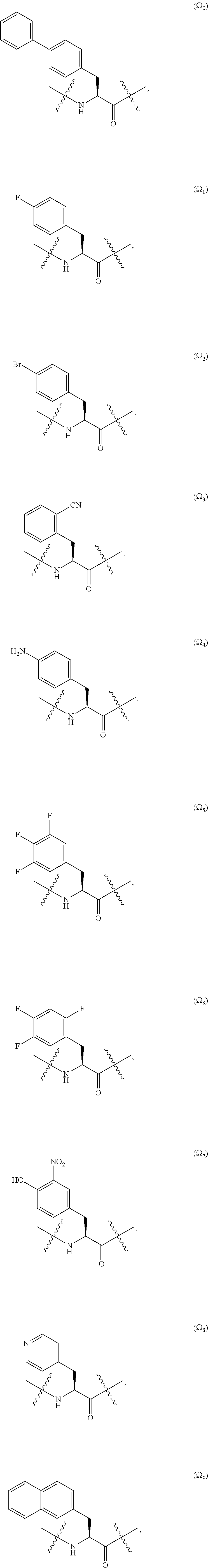

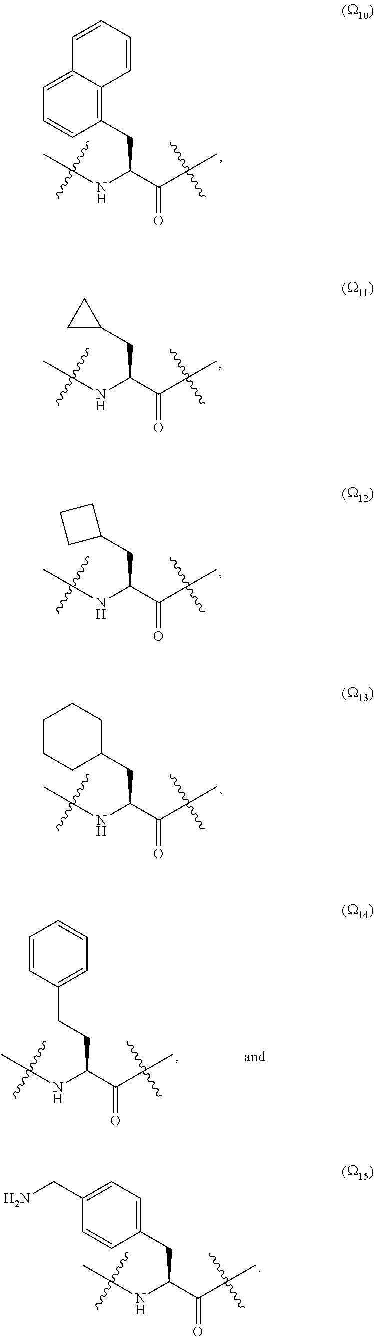 [SMILES: *N[C@@H](Cc1cccc2ccccc12)C(*)=O ; *N[C@@H](CC1CC1)C(*)=O ; *N[C@@H](CC1CCC1)C(*)=O ; *N[C@@H](CC1CCCCC1)C(*)=O ; *N[C@@H](CCc1ccccc1)C(*)=O ; *N[C@@H](Cc1ccc(CN)cc1)C(*)=O]

In some embodiments, the one or more peptides comprise a sequence selected from the group consisting of

(SEQ ID NO: 3)
IEEQAKTFLDKFNHEAEDLFYQS;
(SEQ ID NO: 45)
IEEQAKTFLDKFNHEAEDLFYQSSLAS;
(SEQ ID NO: 46)
QSTIEEQAKTFLDKF;
(SEQ ID NO: 47)
IEEQAKTFLDKFNHE;
(SEQ ID NO: 48)
QAKTFLDKFNHEAED;
(SEQ ID NO: 49)
TFLDKFNHEAEDLFY;
(SEQ ID NO: 50)
DKFNHEAEDLFYQSS;
(SEQ ID NO: 51)
NHEAEDLFYQSSLAS;
(SEQ ID NO: 52)
QSTIEEQAKTFLDKFNHEAEDLFYQSSLASWNYNTNITEENVQNMNNAGDK
WSAFLKEQSTLAQMYPLQEIQNLTVKLQLQALQQNGS;
(SEQ ID NO: 53)
IEEQAKLFLDKFNHEAEDLFYQS;
(SEQ ID NO: 54)
IEEQAKDFLDKFNHEAEDLFYQS;
(SEQ ID NO: 55)
IEEQAKYFLDKFNHEAEDLFYQS;
(SEQ ID NO: 56)
IEEQAKTFLDWFNHEAEDLFYQS;
(SEQ ID NO: 57)
IEEQAKTFLEKFNHEAEDLFYQS;
(SEQ ID NO: 58)
IEEQKKTFEDKFNHEAEDLFYQS;
(SEQ ID NO: 59)
IEEQAKTELDKKNHEAEDLFYQS;
(SEQ ID NO: 60)
IEEQAKTFEDKFKHEAEDLFYQS;
(SEQ ID NO: 61)
IEEQAKTFLDKFKHEEEDKKYQS;
(SEQ ID NO: 62)
IEEQKKTEEDKKKHEEEDKKYQS;
(SEQ ID NO: 29)
IEEQAibKTFLDKFNHEAEDLFYQS;
(SEQ ID NO: 32)
IEEQAKTFLDKFNHEAibEDLFYQS;
(SEQ ID NO: 63)
IEEQAKTAibLDKFNHEAEDLFYQS;
(SEQ ID NO: 31)
IEEQAKTFLDKAIbNHEAEDLFYQS;
(SEQ ID NO: 64)
IEEQAKTFLDKFNHEAEDAibFYQS;
(SEQ ID NOD 33)
IEEQAKTFLDKFNHEAEDLAibYQS;
(SEQ ID NO: 65)
IEEQAKTFLDKFNHEAEDLFYQAib;
(SEQ ID NO: 66)
FLDWFNHEAEDLFY;
(SEQ ID NO: 67)
LDWFNHEAEDLFY;
(SEQ ID NO: 68)
DWFNHEAEDLFY;
(SEQ ID NO: 69)
TFLDWFNHEAEDLF;
(SEQ ID NO: 70)
TFLDWFNHEAEDL;
(SEQ ID NO: 71)
TFLDWFNHEAEDLFY;
(SEQ ID NO: 72)
TFLCyaWFHEEAEDLFY;
(SEQ ID NO: 73)
TFLDWFNHEAibEDLFY;
(SEQ ID NO: 74)
TFLCyaWFNHEAibEDLFY;
(SEQ ID NO: 75)
TFLDWFNVEAEDLFY;
(SEQ ID NO: 76)
TFLDWFNFfEAEDLFY;
(SEQ ID NO: 77)
TFLDWFDHEAEDLFY;
(SEQ ID NO: 78)
AFLDKFNHEAEDLFY;
(SEQ ID NO: 79)
TALDKFNHEAEDLFY;
(SEQ ID NO: 80)
TFADKFNHEAEDLFY;
(SEQ ID NO: 81)
TFLAKFNHEAEDLFY;
(SEQ ID NO: 82)
TFLDAFNHEAEDLFY;
(SEQ ID NO: 83)
TFLDKANHEAEDLFY;
(SEQ ID NO: 84)
TFLDKFAHEAEDLFY;
(SEQ ID NO: 85)
TFLDKFNAEAEDLFY;
(SEQ ID NO: 86)
TFLDKFNHAAEDLFY;
(SEQ ID NO: 87)
TFLDKFNHEAADLFY;
(SEQ ID NO: 88)
TFLDKFNHEAEALFY;
(SEQ ID NO: 89)
TFLDKFNHEAEDAFY;
(SEQ ID NO: 90)
TFLDKFNHEAEDLAY;
(SEQ ID NO: 91)
TFLDKFNHEAEDLFA;
(SEQ ID NO: 92)
EEQAKTFLDKFNHEAEDLFYQSSGGLGKGDFR;
(SEQ ID NO: 93)
EEQAKTFLDKFNHEAEDLFYQSSpLGKGDFR;
(SEQ ID NO: 94)
EEQAKTFLDKFNHEAEDLFYQSSkLGKGDFR;
(SEQ ID NO: 95)
EEQAKTFLCyaKFNHEAEDLFYQSSGLGKGDFR;
(SEQ ID NO: 96)
EEQAKTFLDKFNHEAEDLFYQSSGLGKGCyaFR;
(SEQ ID NO: 97)
EEQAKTFLDKFNFfEAEDLFYQSSGLGKGDFR;
(SEQ ID NO: 98)
EEQAKTFLDKFNVEAEDLFYQSSGLGKGDFR;
(SEQ ID NO: 101)
AQMYPLQEGIEEQAKTFLDKFNHEAEDLFYQSSGLGKGDFR;
(SEQ ID NO: 102)
AQMYPLQEGGIEEQAKTFLDKFNHEAEDLFYQSSGLGKGDFR;
(SEQ ID NO: 103)
AQMYPLQEGIEEQAKTFLDKFNHEAEDLFYQSS;
(SEQ ID NO: 104)
YFLDWFNHEAEDLFY;
(SEQ ID NO: 105)
TFLDWFNHEAEDKFY;
(SEQ ID NO: 106)
TFLDWFNAEAEDLFY;
(SEQ ID NO: 107)
TFLDWFNAibEAEDLFY;
(SEQ ID NO: 108)
TFLDWFNHEAEDOrnFY;
(SEQ ID NO: 109)
TFLDWFNHEAEDPipFY;
(SEQ ID NO: 110)
TFLDWFNHEPipEDLFY;
(SEQ ID NO: 111)
TFLDWFNPipEAEDLFY;
(SEQ ID NO: 112)
TFLDWFNHEAEDLFYK;
(SEQ ID NO: 113)
SLDQINVTFLDLEYEMKKLEEAIKKLEESYIDLKELGSGSGSGSGSTF
LDWFNHEAEDLFY;
(SEQ ID NO: 114)
SLDQINVTFLDLEYEMKKLEEAIKKLEESYIDLKELGSGRRARSGSTF
LDWFNHEAEDLFY;
(SEQ ID NO: 115)
TFLDΩ₀FNHEAEDLFY;
(SEQ ID NO: 116)
TFLDΩ₁FNHEAEDLFY;
(SEQ ID NO: 117)
TFLDΩ₂FNHEAEDLFY;
(SEQ ID NO: 118)
TFLDΩ₃FNHEAEDLFY;
(SEQ ID NO: 119)
TFLDΩ₄FNHEAEDLFY;
(SEQ ID NO: 120)
TFLDΩ₅FNHEAEDLFY;
(SEQ ID NO: 121)
TFLDΩ₆FNHEAEDLFY;
(SEQ ID NO: 122)
TFLDΩ₇FNHEAEDLFY;
(SEQ ID NO: 123)
TFLDΩ₈FNHEAEDLFY;
(SEQ ID NO: 124)
TFLDΩ₉FNHEAEDLFY;
(SEQ ID NO: 125)
TFLDΩ₁₀FNHEAEDLFY;
(SEQ ID NO: 126)
TFLDΩ₁₁FNHEAEDLFY;
(SEQ ID NO: 127)
TFLDΩ₁₂FNHEAEDLFY;
(SEQ ID NO: 128)
TFLDΩ₁₃FNHEAEDLFY;
(SEQ ID NO: 129)
TFLDΩ₁₄FNHEAEDLFY;
and
(SEQ ID NO: 130)
TFLDΩ₁₅FNHEAEDLFY.

In some embodiments, the one or more peptides comprise a sequence selected from the group consisting of

(SEQ ID NO: 99)
GLGKGDFR;
and
(SEQ ID NO: 100)
AQMYPLQEG.

This disclosure further provides a multivalent CoV-2 spike binding peptide have a structure according to Formula (III):

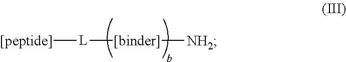

wherein L is an alkyl or heteroalkyl linker between 4 Å and 50 Å in length or L is absent;
[binder] for each occurrence is independently selected from the group consisting of

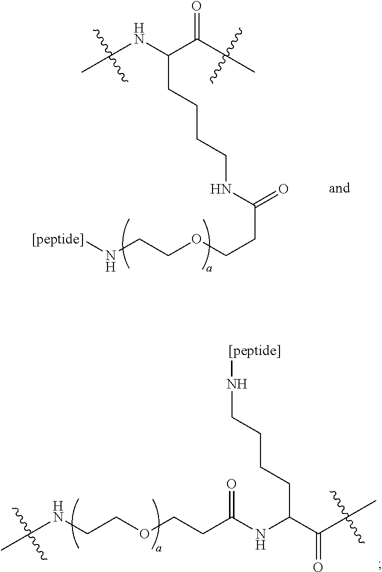

a is for each occurrence independently 1-12;
b is 1-9; and
[peptide], for each occurrence, independently comprises the formula:

(SEQ ID NO: 143)
X₀X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂
X₂₃X₂₄;

wherein:
X₀ is QST, AQMYPLQEG (SEQ ID NO: 135), AQMYPLQEGG (SEQ ID NO: 136), or absent;
X₁ is I or absent;
X₂ is E or absent;
X₃ is E or absent;
X₄ is Q or absent;
X₅ is A, K, or 2-amino isobutyric acid (Aib), absent, or X₅ taken together with X₈ or X₁₂ is represented by Formula (I):

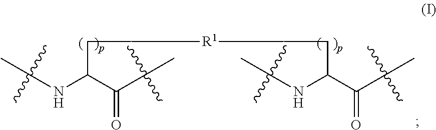

X₆ is K, K*, A, S, Q, H, T, or absent;
X₇ is T, L, D, Y, A, Aib, or absent;
X₈ is F, S, T, H, N, D, E, A, Aib, absent, or X₈ taken together with X₅, X₁₂, or X₁₆ is represented by Formula (I);
X₉ is L, E, A, or absent;
X₁₀ is D, E, A, cysteic acid (Cya), or absent;
X₁₁ is K, Q, W, A, absent, or is represented by Formula (II):

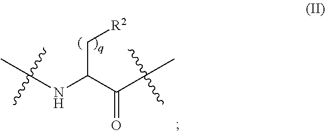

X₁₂ is F, K, A, Aib, absent, or X₁₂ taken together with X₅, X₈, or X₁₆ is represented by Formula (I);
X₃ is N, K, H, D, A, or absent;
X₁₄ is H, E, V, A, Aib, 4-aminopiperidine-4-carboxylic acid (Pip), 4-fluorophenylalanine (Ff), or absent;
X₁₅ is E, A, or absent;
X₁₆ is A, E, Aib, Pip, absent, or X₁₆ taken together with X₈, X₁₂, or X₂₀ is represented by Formula (I);
X₁₇ is E, A, or absent;
X₁₈ is D, A, or absent;
X₁₉ is L, K, A, Aib, omithine (Orn), Pip or absent;
X₂₀ is F, K, A, Aib, absent, or X₂₀ taken together with X₁₆ is represented by Formula (I);
X₂₁ is Y, A, or absent;
X₂₂ is Q, K, or absent;
X₂₃ is S, Aib or absent;
X₂₄ is S, SLAS (SEQ ID NO: 137), SGGLGKGDFR (SEQ ID NO: 138), SpLGKGDFR (SEQ ID NO: 139), SkLGKGDFR (SEQ ID NO: 140), SGLGKGDFR (SEQ ID NO: 141), SGLGKGCyaFR (SEQ ID NO: 142), or absent;
wherein when any of X₁ through X₁₂ are absent, all residues prior to the absent residue are also absent;
wherein when any of X₁₃ through X₂₃ are absent, all residues subsequent to the absent residue are also absent;
wherein at least 12 consecutive residues are not absent;
wherein K* represents a lysine residue covalently bound at its ζ-nitrogen with C(O)(CH₂)ₘCH₃ or

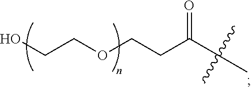

wherein the N-terminal amino group of the peptide is optionally substituted with C(O)(CH₂)ₘCH₃ or

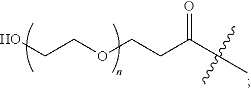

wherein the C-terminus of the peptide forms an amide bond with an amino group of L or [binder] or wherein the C-terminus of the peptide is covalently attached to [binder] via a heteroaryl group;

wherein R¹ is —NHC(O)—, —S—S—, —S-(aryl)-S—, —S-(aryl)-(aryl)-S—, —S-(perfluoroaryl)-S—, —S-(perfluoroaryl)-(perfluoroaryl)-S— —S—(C₁₋₆ alkyl)-S—, —S—CH₂—C(O)—CH₂—S—, —NH-(aryl)-NH—, —NHC(O)-(aryl)-C(O)NH—, —NHC(O)-(aryl)-(aryl)-C(O)NH—, —NHC(O)-(perfluoroaryl)-C(O)NH—, —NHC(O)-(perfluoroaryl)-(perfluoroaryl)-C(O)NH—, —NHC(O)—(C₁₋₆ alkyl)-C(O)NH—, —NHC(O)—CH₂—C(O)—CH₂—C(O)NH—, C₂₋₆ alkenyl, or heteroaryl; wherein R² is aryl, heteroaryl, or C₃₋ₛ cycloalkyl, each of which is optionally substituted with 1, 2, or substituents independently selected from the group consisting of C₁₋₆ alkyl, aryl, nitro, halo, cyano, amino, hydroxy, and C₁₋₃ alkylamino;
m is for each occurrence independently 0-16;
n is for each occurrence independently 4-1000; and
p is for each occurrence independently 1-6;
q is 1-3.

In some embodiments, L is

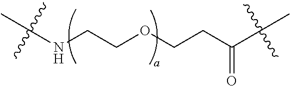

In some embodiments, the C-terminus of the peptide forms an amide bond with an amino group of L or [binder].

In some embodiments, the C-terminus of the peptide is covalently attached to [binder] via a 1,2,3-triazole.

In some embodiments, the multivalent CoV-2 spike binding peptide has a structure according to Formula (IV):

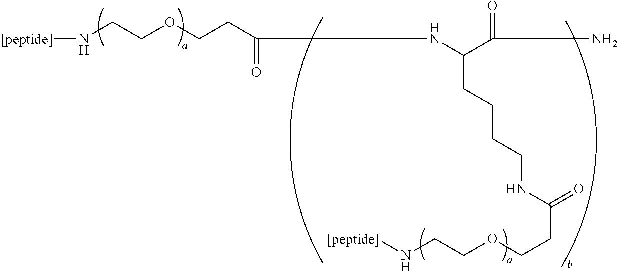

wherein, a, b, and [peptide] are as defined previously

In some embodiments, the multivalent Cov-2 spike binding protein has the following structure:

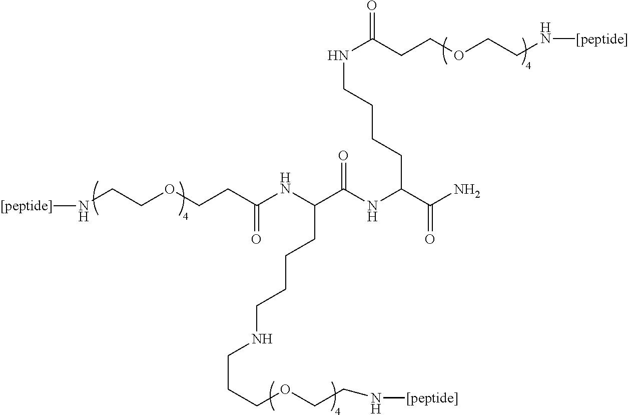

In some embodiments, the multivalent CoV-2 spike binding peptide has a structure according to Formula (V):

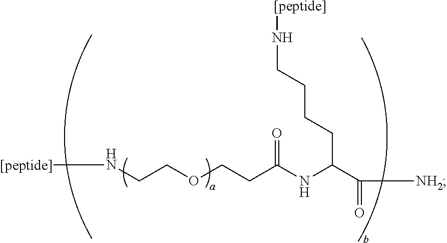

wherein, a, b, and [peptide] are as defined previously

In some embodiments, the multivalent Cov-2 spike binding protein has a structure selected from the group consisting of:

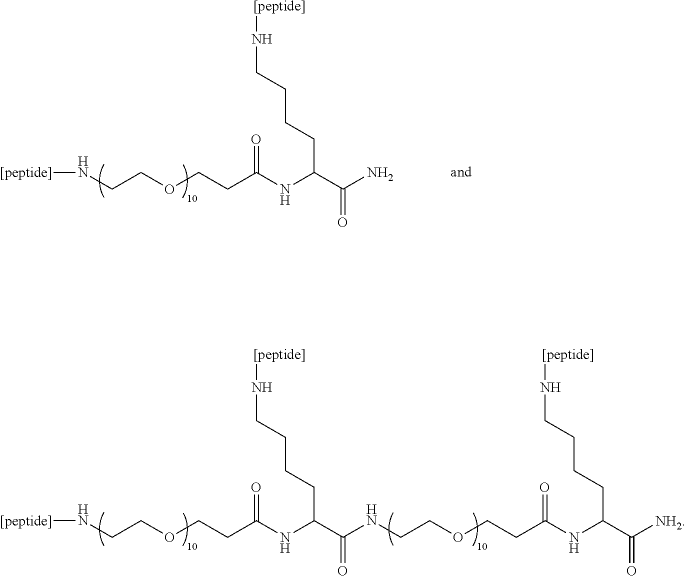

In some embodiments, the [peptide] is selected from the group consisting of

(SEQ ID NO: 3)
IEEQAKTFLDKFNHEAEDLFYQS,
(SEQ ID NO: 4)
IEEQAKTFLDKFNHEAEDLFYQ,
(SEQ ID NO: 5)
IEEQAKTFLDKFNHEAEDLFY,
(SEQ ID NO: 6)
IEEQAKTFLDKFNHEAEDLF,
(SEQ ID NO: 7)
IEEQAKTFLDKFNHEAEDL,
(SEQ ID NO: 8)
IEEQAKTFLDKFNHEAED,
(SEQ ID NO: 9)
EEQAKTFLDKFNHEAEDLFYQS,
(SEQ ID NO: 10)
EQAKTFLDKFNHEAEDLFYQS,
(SEQ ID NO: 11)
QAKTFLDKFNHEAEDLFYQS,
(SEQ ID NO: 12)
AKTFLDKFNHEAEDLFYQS,
(SEQ ID NO: 13)
KTFLDKFNHEAEDLFYQS,
(SEQ ID NO: 14)
TFLDKFNHEAEDLFYQS,
(SEQ ID NO: 17)
IEEQAK*TFLDKFNHEAEDLFYQS,
(SEQ ID NO: 18)
IEEQAATFLDKFNHEAEDLFYQS,
(SEQ ID NO: 19)
IEEQASTFLDKFNHEAEDLFYQS,
(SEQ ID NO: 20)
IEEQAQTFLDKFNHEAEDLFYQS,
(SEQ ID NO: 21)
IEEQAKTFLDKFNHEAEDLFYQS,
(SEQ ID NO: 22)
IEEQATTFLDKFNHEAEDLFYQS,
(SEQ ID NO: 23)
IEEQAKTSLDKFNHEAEDLFYQS,
(SEQ ID NO: 24)
IEEQAKTTLDKFNHEAEDLFYQS,
(SEQ ID NO: 25)
IEEQAKTHLDKFNHEAEDLFYQS,
(SEQ ID NO: 26)
IEEQAKTNLDKFNHEAEDLFYQS,
(SEQ ID NO: 27)
IEEQAKTDLDKFNHEAEDLFYQS,
(SEQ ID NO: 28)
IEEQAKTFLDΩFNHEAEDLFYQS,
(SEQ ID NO: 29)
IEEQAibKTFLDKFNHEAEDLFYQS,
(SEQ ID NO: 30)
IEEQAKAibFLDKFNHEAEDLFYQS,
(SEQ ID NO: 31)
IEEQAKTFLDKAibNHEAEDLFYQS,
(SEQ ID NO: 32)
IEEQAKTFLDKFNHEAibEDLFYQS,
(SEQ ID NO: 33)
IEEQAKTFLDKFNHEAEDLAibYQS,
(SEQ ID NO: 16)
STIEEQAKTFLDKFNHEAEDLFYQSSLASWNYNTNITEENVQNMNNAGDKW
SAFLKEQSTLAQMYPLQEIQ,
(SEQ ID NO: 42)
STIEEQAKTFLDKFNHEAEDLFYQSSLASWNYNTNITEENVQNMNNAGDKW
SAFLKEQSTLAQMYPLQEIQNLTVKLQLQALQQN;
(SEQ ID NO: 131)
IEEQAKTFLDKFNHEAEDLFYQSS
(SEQ ID NO: 45)
IEEQAKTFLDKFNHEAEDLFYQSSLAS;
(SEQ ID NO: 46)
QSTIEEQAKTFLDKF;
(SEQ ID NO: 47)
IEEQAKTFLDKFNHE;
(SEQ ID NO: 48)
QAKTFLDKFNHEAED;
(SEQ ID NO: 49)
TFLDKFNHEAEDLFY;
(SEQ ID NO: 50)
DKFNHEAEDLFYQSS;
(SEQ ID NO: 51)
NHEAEDLFYQSSLAS;
(SEQ ID NO: 52)
QSTIEEQAKTFLDKFNHEAEDLFYQSSLASWNYNTNITEENVQNMNNAGDK
WSAFLKEQSTLAQMYPLQEIQNLTVKLQLQALQQNGS;
(SEQ ID NO: 53)
IEEQAKLFLDKFNHEAEDLFYQS;
(SEQ ID NO: 54)
IEEQAKDFLDKFNHEAEDLFYQS;
(SEQ ID NO: 55)
IEEQAKYFLDKFNHEAEDLFYQS;
(SEQ ID NO: 56)
IEEQAKTFLDWFNHEAEDLFYQS;
(SEQ ID NO: 57)
IEEQAKTFLEKFNHEAEDLFYQS;
(SEQ ID NO: 58)
IEEQKKTFEDKFNHEAEDLFYQS;
(SEQ ID NO: 59)
IEEQAKTELDKKNHEAEDLFYQS;
(SEQ ID NO: 60)
IEEQAKTFEDKFKHEAEDLFYQS;
(SEQ ID NO: 61)
IEEQAKTFLDKFKHEEEDKKYQS;
(SEQ ID NO: 62)
IEEQKKTEEDKKKHEEEDKKYQS;
(SEQ ID NO: 63)
IEEQAKTAibLDKFNHEAEDLFYQS;
(SEQ ID NO: 64)
IEEQAKTFLDKFNHEAEDAibFYQS;
(SEQ ID NO: 65)
IEEQAKTFLDKFNHEAEDLFYQAib;
(SEQ ID NO: 66)
FLDWFNHEAEDLFY;
(SEQ ID NO: 67)
LDWFNHEAEDLFY;
(SEQ ID NO: 68)
DWFNHEAEDLFY;
(SEQ ID NO: 69)
TFLDWFNHEAEDLF;
(SEQ ID NO: 70)
TFLDWFNHEAEDL;
(SEQ ID NO: 71)
TFLDWFNHEAEDLFY;
(SEQ ID NO: 72)
TFLCyaWFHEEAEDLFY;
(SEQ ID NO: 73)
TFLDWFNHEAibEDLFY;
(SEQ ID NO: 74)
TFLCyaWFNHEAibEDLFY;
(SEQ ID NO: 75)
TFLDWFNVEAEDLFY;
(SEQ ID NO: 76)
TFLDWFNFfEAEDLFY;
(SEQ ID NO: 77)
TFLDWFDHEAEDLFY;
(SEQ ID NO: 78)
AFLDKENHEAEDLFY;
(SEQ ID NO: 79)
TALDKFNHEAEDLFY;
(SEQ ID NO: 80)
TFADKENHEAEDLFY;
(SEQ ID NO: 81)
TFLAKFNHEAEDLFY;
(SEQ ID NO: 82)
TFLDAFNHEAEDLFY;
(SEQ ID NO: 83)
TFLDKANHEAEDLFY;
(SEQ ID NO: 84)
TFLDKFAHEAEDLFY;
(SEQ ID NO: 85)
TFLDKFNAEAEDLFY;
(SEQ ID NO: 86)
TFLDKFNHAAEDLFY;
(SEQ ID NO: 87)
TFLDKFNHEAADLFY;
(SEQ ID NO: 88)
TFLDKFNHEAEALFY;
(SEQ ID NO: 89)
TFLDKFNHEAEDAFY;
(SEQ ID NO: 90)
TFLDKFNHEAEDLAY;
(SEQ ID NO: 91)
TFLDKFNHEAEDLFA;
(SEQ ID NO: 92)
EEQAKTFLDKFNHEAEDLFYQSSGGLGKGDFR;
(SEQ ID NO: 93)
EEQAKTFLDKFNHEAEDLFYQSSpLGKGDFR;
(SEQ ID NO: 94)
EEQAKTFLDKFNHEAEDLFYQSSkLGKGDFR;
(SEQ ID NO: 95)
EEQAKTFLCyaKFNHEAEDLFYQSSGLGKGDFR;
(SEQ ID NO: 96)
EEQAKTFLDKFNHEAEDLFYQSSGLGKGCyaGFR;
(SEQ ID NO: 97)
EEQAKTFLDKFNFfEAEDLFYQSSGLGKGDFR;
(SEQ ID NO: 98)
EEQAKTFLDKFNVEAEDLFYQSSGLGKGDFR;
(SEQ ID NO: 101)
AQMYPLQEGIEEQAKTFLDKFNHEAEDLFYQSSGLGKGDFR;
(SEQ ID NO: 102)
AQMYPLQEGGIEEQAKTFLDKFNHEAEDLFYQSSGLGKGDFR;
(SEQ ID NO: 103)
AQMYPLQEGIEEQAKTFLDKFNHEAEDLFYQSS;
(SEQ ID NO: 104)
YFLDWFNHEAEDLFY;
(SEQ ID NO: 105)
TFLDWFNHEAEDKFY;
(SEQ ID NO: 106)
TFLDWFNAEAEDLFY;
(SEQ ID NO: 107)
TFLDWFNAibEAEDLFY;
(SEQ ID NO: 108)
TFLDWFNHEAEDOrnFY;
(SEQ ID NO: 109)
TFLDWFNHEAEDPipFY;
(SEQ ID NO: 110)
TFLDWFNHEPipEDLFY;
(SEQ ID NO: 111)
TFLDWFNPipEAEDLFY;
(SEQ ID NO: 112)
TFLDWFNHEAEDLFYK;
(SEQ ID NO: 113)
SLDQINVTFLDLEYEMKKLEEAIKKLEESYIDLKELGSGSGSGSGSTFLDWFN
HEAEDLFY;
(SEQ ID NO: 114)
SLDQINVTFLDLEYEMKKLEEAIKKLEESYIDLKELGSGRRARSGSTFLDWFN
HEAEDLFY;
(SEQ ID NO: 115)
TFLDΩ₀FNHEAEDLFY;
(SEQ ID NO: 116)
TFLDΩ₁FNHEAEDLFY;
(SEQ ID NO: 117)
TFLDΩ₂FNHEAEDLFY;
(SEQ ID NO: 118)
TFLDΩ₃FNHEAEDLFY;
(SEQ ID NO: 119)
TFLDΩ₄FNHEAEDLFY;
(SEQ ID NO: 120)
TFLDΩ₅FNHEAEDLFY;
(SEQ ID NO: 121)
TFLDΩ₆FNHEAEDLFY;
(SEQ ID NO: 122)
TFLDΩ₇FNHEAEDLFY;
(SEQ ID NO: 123)
TFLDΩ₈FNHEAEDLFY;
(SEQ ID NO: 124)
TFLDΩ₉FNHEAEDLFY;
(SEQ ID NO: 125)
TFLDΩ₁₀FNHEAEDLFY;
(SEQ ID NO: 126)
TFLDΩ₁₁FNHEAEDLFY;
(SEQ ID NO: 127)
TFLDΩ₁₂FNHEAEDLFY;
(SEQ ID NO: 128)
TFLDΩ₁₃FNHEAEDLFY;
(SEQ ID NO: 129)
TFLDΩ₁₄FNHEAEDLFY;
and
(SEQ ID NO: 130)
TFLDΩ₁₅FNHEAEDLFY.
In some embodiments, [peptide] is selected from the group consisting of
(SEQ ID NO: 131)
IEEQAKTFLDKFNHEAEDLFYQSS
and
(SEQ ID NO: 132)
TFLDWFNHEAEDLFY.

In some embodiments, [peptide] is selected from the group consisting of IEEQAKTFLDKFNHEAEDLFYQSS (SEQ ID NO: 131) and TFLDWFNHEAEDLFY (SEQ ID NO: 132).

In some embodiments, a composition comprises the multivalent Cov-2 spike binding protein. In some embodiments, the multivalent Cov-2 spike binding protein specifically binds to a receptor binding domain (RBD) of the spike protein. In some embodiments, the multivalent Cov-2 spike binding protein specifically binds to human ACE2 receptor. In some embodiments, the coronavirus is selected from the group consisting of human coronavirus 229E (HCoV-229E), human coronavirus OC43 (HCoV-OC43), severe acute respiratory syndrome coronavirus (SARS-CoV), human coronavirus NL63 (HCoV-NL63), human coronavirus HKU1, Middle East respiratory syndrome-related coronavirus (MERS-CoV) and severe acute respiratory syndrome coronavirus 2 (SARS-CoV-2). The composition of claim 39, wherein the coronavirus is SARS-CoV-2.

In some embodiments, the peptide binds the spike protein receptor binding domain of the coronavirus with a KD of less than about 300 nM.

Further provided is a method reducing probability of viral infection in a human subject, comprising administering a therapeutically effective amount of the pharmaceutical composition described herein.

In some embodiments, the viral infection is caused by a coronavirus. In some embodiments, the coronavirus is selected from the group consisting of HCoV-229E, HCoV-OC43, SARS-CoV, HCoV-NL63, human coronavirus HKU1, MERS-CoV and SARS-CoV-2.

In some embodiments, the coronavirus is SARS-CoV-2.

In some embodiments, the administration of the pharmaceutical composition comprises intradermal, intramuscular, intraperitoneal, intravenous, subcutaneous, intranasal, epidural, sublingual, intracerebral, intravaginal, transdermal, rectal, by inhalation, or topical administration.

In some embodiments, the human subject is at least 50 years of age.

The present disclosure further provides a method of detecting the presence of a viral infection in a human subject in need thereof, comprising contacting a sample from the subject with the composition of any one of claims 1-24 or 35-40, detecting specific binding between the composition and an antibody that specifically binds the infecting virus, wherein specific binding between the antibody and the composition indicates the presence of viral infection in the subject.

In some embodiments, the composition is covalently bound to a solid phase substrate. In some embodiments, wherein the detection of the detecting specific binding between the composition and the virus is performed using an immunoassay. In some embodiments the immunoassay is an enzyme-linked immunosorbent assay (ELISA).

In some embodiments, the viral infection is caused by a coronavirus. In some embodiments, the coronavirus is selected from the group consisting of HCoV-229E, HCoV-OC43, SARS-CoV, HCoV-NL63, human coronavirus HKU1, MERS-CoV and SARS-CoV-2. In some embodiments, the coronavirus is SARS-CoV-2. In some embodiments, the subject may not have a symptom, or may have a symptom associated with viral infection selected from the group consisting of fever, cough, shortness of breath, pain or pressure in the chest, confusion, bluish lips or face, pneumonia, bronchitis, runny nose, sneezing, chills, exacerbated asthma, acute respiratory distress syndrome (ARDS), RNAaemia, acute cardiac injury, shock, myalgia, fatigue, sputum production, rusty colored sputum, bloody sputum, swelling of lymph nodes, middle ear infection, joint pain, wheezing, headache, hemoptysis, diarrhea, dyspnea, redness, swelling or edema, pain, loss of function, organ dysfunction, multi-organ system failure, acute kidney injury, malnutrition, sepsis, hypotension, hypertension, hypothermia, hypoxemia, leukocytosis, leukopenia, lymphopenia, thrombocytopenia, nasal congestion, sore throat, unwillingness to drink, convulsions, ongoing vomiting, abdominal pain, secondary infection, cytokine release syndrome, and multi-organ failure. In some embodiments, the human subject is at least 50 years of age.

The present disclosure further provides a kit comprising any of the compositions described herein and a solid phase substrate. In some embodiments, the kit also includes a monoclonal antibody that specifically binds to a human Ig constant domain. In some embodiments, the monoclonal antibody comprises a detectable marker.

### BRIEF DESCRIPTION OF THE DRAWINGS

FIG. 1 is the total ion chromatogram (TIC) of purified biotinylated (A) ACE2-h1 and (B) ACE2-h2 peptides.

FIG. 2 is a graphic representation of the results of a 200 ns molecular dynamics simulation of human ACE2-PD domain α-helix 1 and the SARS-CoV-2 S protein receptor binding domain (RBD) complex. FIG. 2A is the root-mean-square deviation (RMSD) from residue 1 to 23; FIG. 2B is the per residue heatmap along 200 ns simulation trajectory;

FIG. 2C is the binding interface between SARS-CoV-2-S and ACE2-h1 peptide after 200 ns simulation.

FIG. 3 is graphic representation of bio-layer interferometry testing the interaction of lead peptides and SARS-CoV-2-RBD. FIG. 3A is the assay result of ACE2-h1 peptide (SEQ ID NO. 3) binding to SARS-CoV-2-RBD; FIG. 3B is the assay result of ACE2-h2 peptide (SEQ ID NO: 15) binding to SARS-CoV-2-RBD.

FIG. 4 is graphic representation of bio-layer interferometry testing the interaction of lead peptides and an unrelated human protein. FIG. 4A is the assay result of ACE2-h1 peptide (SEQ ID NO: 3) binding to an unrelated human protein; FIG. 4B is the assay result of ACE2-h2 peptide (SEQ ID NO: 15) binding to an unrelated human protein.

FIG. 5 is a schematic representation of the structures of t1SBP1 and t2SBP1. Figure discloses “IEEQAKTFLDKFNHEAEDLFYQSS” as SEQ ID NO: 131.

### DETAILED DESCRIPTION

The novel coronavirus SARS-CoV-2 spike protein binds to the human ACE2 protein on the cell's surface as a mechanism to enter cells and to cause severe respiratory diseases in most instances. The disclosure herein provides peptides, which can specifically bind the spike protein (S) of the SARS-CoV-2 coronavirus, thereby blocking the potential interaction of the coronavirus spike protein with the human ACE2 receptor. Blocking this interaction should reduce the ability of the coronavirus to infect host cells.

Also provided herein is a method of reducing the probability of having viral infection in a human subject by administrating the novel peptides described herein. In some embodiments, the peptides are effective in treating or preventing the disease caused by severe acute respiratory syndrome coronavirus 2 (SARS-CoV-2).

Coronaviruses (CoVs) are the largest group of viruses belonging to the Nidovirales order, which includes Coronaviridae, Arteriviridae, and Roniviridae families. Coronaviruses are further subdivided into four groups, the alpha, beta, gamma and delta coronaviruses. The viruses were initially sorted into these groups based on serology but are now divided by phylogenetic clustering (Fehr et al., Methods Mol Biol. 2015; 1282: 1-23).

Coronaviruses are enveloped, non-segmented positive-sense RNA viruses, which contain approximately 30 kilobase (kb) genomes. Other features of coronaviruses include: i) a highly conserved genomic organization, with a large replicase gene preceding structural and accessory genes; ii) expression of many nonstructural genes by ribosomal frameshifting; iii) several unique or unusual enzymatic activities encoded within the large replicase-transcriptase polyprotein; and iv) expression of downstream genes by synthesis of 3′ nested sub-genomic mRNAs.

Coronavirus virions are spherical with diameters of approximately 125 nm (Barcena M et al., PNAS, 2009; 106(2):582-587; Neuman et al., J Virol. 2006; 80(16):7918-7928). The most prominent feature of coronaviruses is the club-shape spike projections emanating from the surface of the virion. These spikes are a defining feature of the virion and give them the appearance of a solar corona, prompting the name coronaviruses. Within the envelope of the virion is the nucleocapsid. Coronaviruses have helically symmetrical nucleocapsids, which are uncommon among positive-sense RNA viruses, but far more common for negative-sense RNA viruses. Coronavirus virus particles contain four main structural proteins. These are the spike (S), membrane (M), envelope (E), and nucleocapsid (N) proteins, all of which are encoded within the 3′ end of the viral genome. The S protein (˜150 kDa), utilizes an N-terminal signal sequence to gain access to the ER, and is heavily N-linked glycosylated. Homotrimers of the virus encoded S protein make up the distinctive spike structure on the surface of the virus (Beniac et al., Nat Struct Mol Biol., 2006; 13(8):751-752; Delmas et al., J Virol. 1990; 64(11):5367-5375). The trimeric S glycoprotein is a class I fusion protein (Bosch et al., J Virol. 2003; 77(16):8801-8811.) and mediates attachment to the host receptor (Collins et al., Virology. 1982; 119(2):358-371]. In most, but not all, coronaviruses, S is cleaved by a host cell furin-like protease into two separate polypeptides noted S1 and S2 (Abraham et al., Virology. 1990; 176(1):296-301.; Luytjes et al., Virology. 1987; 161(2):479-487.). S1 contains the large receptor binding domain (RBD) of the S protein while S2 forms the stalk of the spike molecule (De Groot et al., J Mol Biol. 1987, 196(4):963-966).

In the case of SARS-CoV, the spike glycoprotein (S protein) on the virion surface mediates receptor recognition and membrane fusion. During viral infection, the trimeric S protein is cleaved into S1 and S2 subunits and S1 subunits are released in the transition to the post-fusion conformation. S1 contains the receptor binding domain (RBD), which directly binds to the peptidase domain (PD) of ACE2, while S2 is responsible for membrane fusion. When S1 binds to the host receptor ACE2, another cleavage site on S2 is exposed and is cleaved by host proteases, a process that is critical for viral infection. The S protein of SARS-CoV-2 may also exploit ACE2 for host infection. A recent publication reported the structure of the S protein of SARS-CoV-2 and showed that the ectodomain of the SARS-CoV-2 S protein binds to the PD of ACE2 with a dissociation constant (K_{D}) of ˜15 nM. The sequence of SARS-CoV-2 RBD (SEQ ID NO: 1) is listed below:

(SEQ ID NO: 1)
RVQPTESIVREPNITNLCPFGEVFNATREASVYAW
NRKRISNCVADYSVLYNSASFSTFKCYGVSPTKLN
DLCETNVYADSFVIRGDEVRQIAPGQTGKIADYNY
KLPDDETGCVIAWNSNNLDSKVGGNYNYLYRLFRK
SNLKPFERDISTEIYQAGSTPCNGVEGPNCYFPLQ
SYGTQPTNGVGYQPYRVVVLSFELLHAPATVCGPK
KSTNLVKNKCVNE

Seven strains of human coronaviruses are known: human coronavirus 229E (HCoV-229E); human coronavirus OC43 (HCoV-OC43); severe acute respiratory syndrome coronavirus (SARS-CoV); human coronavirus NL63 (HCoV-NL63, New Haven coronavirus); human coronavirus HKU1; middle East respiratory syndrome-related coronavirus (MERS-CoV, previously known as novel coronavirus 2012 and HCoV-EMC); and SARS-CoV-2, previously known as 2019-nCoV or “novel coronavirus 2019”.

In humans, coronaviruses cause respiratory tract infections that can be mild, such as some cases of the common cold and others that can be lethal, such as SARS, MERS, and COVID-19.

Coronavirus disease 2019 (COVID-19) is an infectious disease caused by SARS-CoV-2. Common symptoms include fever, cough and shortness of breath. Muscle pain, sputum production and sore throat are less common. While the majority of cases result in mild symptoms, some progress to severe pneumonia and multi-organ failure. The rate of deaths per number of diagnosed cases is on average 3.4%, ranging from 0.2% in those less than 20 to approximately 15% in those over 80 years old.

Angiotensin-converting enzyme 2 (ACE2) belongs to the angiotensin-converting enzyme family of dipeptidyl carboxydipeptidases and has considerable homology to human angiotensin 1 converting enzyme. The primary physiological role of ACE2 is the maturation of angiotensin, a peptide hormone that controls vasoconstriction and blood pressure. ACE2 catalyzes the cleavage of angiotensin I into angiotensin 1-9, and angiotensin II into the vasodilator angiotensin 1-7. The organ- and cell-specific expression of this gene suggests that it may play a role in the regulation of cardiovascular and renal function, as well as fertility. ACE2 is a type I membrane protein expressed in lungs, heart, kidneys and intestine. Decreased expression of ACE2 is associated with cardiovascular diseases. Full-length ACE2 consists of an N-terminal PD and a C-terminal Collectrin-like domain (CLD) that ends with a single transmembrane helix and a ˜40-residue intracellular segment (15, 21). The PD of ACE2 cleaves angiotensin (Ang) I to produce Ang-(1-9), which is then processed by other enzymes to become Ang-(1-7). ACE2 can also directly process Ang II to give Ang-(1-7). ACE2 is a functional receptor for the spike glycoprotein of the human coronavirus HCoV-NL63 and the human severe acute respiratory syndrome coronaviruses, SARS-CoV and SARS-CoV-2 (COVID-19 virus). It interacts with the RBD domain of coronavirus spike protein through its N terminus PD domain. The sequence of ACE2 (SEQ ID NO: 2) is listed below:

(SEQ ID NO: 2)
MSSSSWLLLSLVAVTAAQSTIEEQAKTFLDKFNHE
AEDLEYQSSLASWNYNTNITEENVQNMNNAGDKWS
AFLKEQSTLAQMYPLQEIQNLTVKLQLQALQQNGS
SVLSEDKSKRLNTILNTMSTIYSTGKVCNPDNPQE
CLLLEPGLNEIMANSLDYNERLWAWESWRSEVGKQ
LRPLYEEYVVLKNEMARANHYEDYGDYWRGDYEVN
GVDGYDYSRGQLIEDVEHTFEEIKPLYEHLHAYVR
AKLMNAYPSYISPIGCLPAHLLGDMWGRFWTNLYS
LTVPFGQKPNIDVTDAMVDQAWDAQRIFKEAEKFF
VSVGLPNMTQGFWENSMLTDPGNVQKAVCHPTAWD
LGKGDFRILMCTKVTMDDFLTAHHEMGHIQYDMAY
AAQPFLLRNGANEGFHEAVGEIMSLSAATPKHLKS
IGLLSPDFQEDNETEINFLLKQALTIVGTLPFTYM
LEKWRWMVFKGEIPKDQWMKKWWEMKREIVGVVEP
VPHDETYCDPASLFHVSNDYSFIRYYTRTLYQFQE
QEALCQAAKHEGPLHKCDISNSTEAGQKLFNMLRL
GKSEPWTLALENVVGAKNMNVRPLLNYFEPLETWL
KDQNKNSFVGWSTDWSPYADQSIKVRISLKSALGD
KAYEWNDNEMYLFRSSVAYAMRQYFLKVKNQMILF
GEEDVRVANIKPRISFNFFVTAPKNVSDIIPRTEV
EKAIRMSRSRINDAFRLNDNSLEFLGIQPTLGPPN
QPPVSIWLIVFGVVMGVIVVGIVILIFTGIRDRKK
KNKARSGENPYASIDISKGENNPGFONTDDVQTSE

### Definitions

As used herein, the term “subject” refers to a mammal. As used, herein, the term, “mammal” includes humans and both domestic animals such as laboratory animals and household pets (e.g., cats, dogs, swine, cattle, sheep, goats, horses, rabbits), and non-domestic animals such as wildlife and the like.

The terms “peptide,” “polypeptide,” and “protein” are used interchangeably herein and refer to a molecule comprising a chain of two or more amino acids (e.g., most typically L-amino acids, but also including, e.g., D-amino acids, modified amino acids, amino acid analogs, and amino acid mimetics).

Naturally-occurring L-amino acids are represented herein by capitalized one-letter amino acid designations, i.e., A, L, M, F, W, K, Q, E, S, P, V, I, C, Y, H, R, N, D, and T. D-amino acids are represented herein by lower-case one-letter amino acid designations, i.e., a, 1, m, f w, k, q, e, s, p, v, i, c, y, h, r, n, d, and t.

Certain non-naturally occurring amino acids used herein are as follows: Aib is α-amino isobutyric acid; Cya is cysteic acid, Ff is 4-fluorophenylalanine, Pip is -aminopiperidine-4-carboxylic acid, and Orn is omithine.

As used herein, the term “alkyl,” means a straight or branched chain hydrocarbon having the number of carbon atoms designated (i.e., C₁-C₆ alkyl means an alkyl having one to six carbon atoms) and includes straight and branched chains. Examples include methyl, ethyl, propyl, isopropyl, butyl, isobutyl, tert butyl, pentyl, neopentyl, and hexyl.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl” means an aromatic carbocyclic system containing 1, 2 or 3 rings, wherein such rings may be fused. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydronaphthalenyl. In one embodiment, “aryl” means phenyl. In some embodiments, aryl groups have 6 carbon atoms. In some embodiments, aryl groups have from six to ten carbon atoms. In some embodiments, aryl groups have from six to sixteen carbon atoms.

As used herein, the term “perfluoroaryl” means an aryl group wherein all hydrogen atoms directly attached to a carbon atom are replaced with fluorine atoms. The term “perfluoroaryl” includes, but is not limited to pentafluorophenyl, divalent tetrafluorophenyl, and heptafluoronaphthyl. In one embodiment, “perfluoroaryl” means pentafluorophenyl. In another embodiment, “perfluoroaryl” means divalent tetrafluorophenyl. In some embodiments, perfluoroaryl groups have from six to ten carbon atoms. In some embodiments, perfluoroaryl groups have from six to sixteen carbon atoms.

As used herein, the term “alkenyl” refers to a hydrocarbon moiety containing, in certain embodiments, from two to six, or two to eight carbon atoms having at least one carbon-carbon double bond. The alkenyl group may or may not be the point of attachment to another group. The term “alkenyl” includes, but is not limited to, ethenyl, 1-propenyl, 1-butenyl, heptenyl, octenyl and the like.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. If the rings are fused, one of the rings must be fully unsaturated and the fused ring(s) may be fully saturated, partially unsaturated or fully unsaturated. The term “heteroaryl” includes, but is not limited to, furanyl, thienyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with an additional group selected from the groups described herein.

As used, herein, the term, “optional” or “optionally” means that the subsequently described event of circumstances may or may not occur, and that the description includes instances where said event or circumstance occurs and instances in which it does not.

As used, herein, the term, “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

As used, herein, the term, “pharmaceutically acceptable salt” includes both acid and base addition salts.

As used, herein, the term, “pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecyl sulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

### DESCRIPTION OF EXEMPLARY EMBODIMENTS

In certain embodiments, the present disclosure relates to isolated peptides that specifically bind to a spike protein of a coronavirus.

In some embodiments, a peptide of the disclosure comprises an amino acid sequence selected from the group consisting of amino acid sequences represented by the consensus sequence of SEQ ID NO: 133:

X₁X₂X₃X₄X₅X₆X₇X₈LDX₁₁X₁₂NHEX₁₆EDX₁₉X₂₀X₂₁X₂₂X₂₃;
wherein:
X₁ is I or absent;
X₂ is E or absent;
X₃ is E or absent;
X₄ is Q or absent;
X₅ is A, Aib, absent, or X₅ taken together with X₈ or X₁₂ is represented by Formula (I):

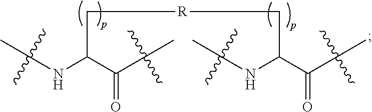

X₆ is K, K*, A, S, Q, H, T, or absent;
X₇ is T or Aib;
X₈ is F, S, T, H, N, D, or X₈ taken together with X₅, X₁₂, or X₁₆ is represented by Formula (I);
X₁₁ is K or Q;
X₁₂ is F, Aib, or X₁₂ taken together with X₅, X₈, or X₁₆ is represented by Formula (I);
X₁₆ is A, Aib, or X₁₆ taken together with X₈, X₁₂, or X₂₀ is represented by Formula (I);
X₁₉ is L or absent;
X₂₀ is F, Aib, absent, or X₂₀ taken together with X₁₆ is represented by Formula (I);
X₂₁ is Y or absent;
X₂₂ is Q or absent;
X₂₃ is S or absent;

wherein when X₂, X₃, X₄, X₅, or X₆ are absent, all previous residues are also absent;

wherein when X₁₉, X₂₀, X₂₁, or X₂₂ are absent, all subsequent residues are also absent;

wherein K* represents a lysine residue covalently bound at its ζ-nitrogen with C(O)(CH₂)ₘCH₃ or

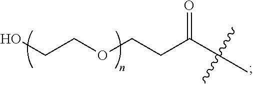

wherein the N-terminal amino group of the peptide is optionally substituted with C(O)(CH₂)ₘCH₃ or

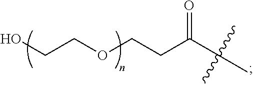

wherein the C-terminus of the peptide comprises a C-terminal acid group or a C-terminal amide group;

wherein R is —NHC(O)—, —S—S—, —S-(aryl)-S—, —S-(aryl)-(aryl)-S—, —S-(perfluoroaryl)-S—, —S-(perfluoroaryl)-(perfluoroaryl)-S— —S—(C₁₋₆ alkyl)-S—, —S—CH₂—C(O)—CH₂—S—, —NH-(aryl)-NH—, —NHC(O)-(aryl)-C(O)NH—, —NHC(O)-(aryl)-(aryl)-C(O)NH—, —NHC(O)-(perfluoroaryl)-C(O)NH—, —NHC(O)-(perfluoroaryl)-(perfluoroaryl)-C(O)NH—, —NHC(O)—(C₁₋₆ alkyl)-C(O)NH—, —NHC(O)—CH₂—C(O)—CH₂—C(O)NH—, C₂₋₆ alkenyl, or heteroaryl;

m is for each occurrence independently 0-16;

n is for each occurrence independently 4-1000; and

p is for each occurrence independently 1-6.

In some embodiments, X₁ is I. In some embodiments, X₁ is absent.

In some embodiments, X₂ is E. In some embodiments, X₂ is absent.

In some embodiments, X₃ is E. In some embodiments X₃ is absent.

In some embodiments, X₄ is Q. In some embodiments, X₄ is absent.

In some embodiments, X₅ is A. In some embodiments, X₅ is Aib. In some embodiments, X₈ is absent. In some embodiments, X₈ taken together with X₈ is represented by Formula (I).

In some embodiments, X₈ taken together with X₂ is represented by Formula (I).

In some embodiments, X₆ is K. In some embodiments, X₆ is K*. In some embodiments, X₆ is A. In some embodiments, X₆ is S. In some embodiments, X₆ is Q. In some embodiments, X₆ is H. In some embodiments, X₆ is T. In some embodiments, X₆ is absent.

In some embodiments, X₇ is T. In some embodiments X₇ is Aib.

In some embodiments, X₈ is F. In some embodiments, X₈ is S. In some embodiments, X₈ is T. In some embodiments, X₈ is H. In some embodiments, X₈ is N. In some embodiments, X₈ is D. In some embodiments, X₈ taken together with X₅ is represented by Formula (I). In some embodiments, X₈ taken together with Xu is represented by Formula (I). In some embodiments, X₈ taken together with X₁₆ is represented by Formula (I).

In some embodiments, X₁₁ is K. In some embodiments, X₁₁ is Q.

In some embodiments, X₁₂ is F. In some embodiments, X₁₂ is Aib. In some embodiments, X₁₂ taken together with X₈ is represented by Formula (I). In some embodiments, X₁₂ taken together with X₈ is represented by Formula (I). In some embodiments, X₁₂ taken together with X₁₆ is represented by Formula (I).

In some embodiments, X₁₆ is A. In some embodiments, X₁₆ is Aib. In some embodiments, X₁₆ taken together with X₈ is represented by Formula (I). In some embodiments, X₁₆ taken together with X₁₂ is represented by Formula (I). In some embodiments, X₁₆ taken together with X₂₀ is represented by Formula (I).

In some embodiments, X₁₉ is L. In some embodiments, X₁₉ is absent.

In some embodiments, X₂₀ is F. In some embodiments, X₂₀ is Aib. In some embodiments, X₂₀ is absent. In some embodiments, X₂₀ taken together with X₁₆ is represented by Formula (I).

In some embodiments, X₂₁ is Y. In some embodiments, X₂₁ is absent.

In some embodiments, X₂₂ is Q. In some embodiments, X₂₂ is absent.

In some embodiments, X₂₃ is S. In some embodiments, X₂₃ is absent.

In some embodiments, m is 0. In some embodiments, m is 12. In some embodiments, m is 14. In some embodiments, m is 16.

In some embodiments, X₁ is I, and X₆ is K. In some embodiments, X₁ is I, and X₇ is T. In some embodiments, X₁ is I, and X₈ is F. In some embodiments, X₁ is I, and X₁₁ is K. In some embodiments, X₁ is I, and X₁₉ is L. In some embodiments, X₁ is I, and X₂₁ is Y. In some embodiments, X₁ is I, and X₂₃ is S.

In some embodiments, X₁ is I, and X₅ taken together with X₈ is represented by Formula (I). In some embodiments, X₁ is I, and X₅ taken together with X₁₂ is represented by Formula (I). In some embodiments, X₁ is I, and X₈ taken together with X₁₂ is represented by Formula (I). In some embodiments, X₁ is I, and X₈ taken together with X₁₆ is represented by Formula (I). In some embodiments, X₁ is I, and X₁₂ taken together with X₁₆ is represented by Formula (I). In some embodiments, X₁ is I, and X₁₆ taken together with X₂₀ is represented by Formula (I).

In some embodiments, X₃ is E, and X₆ is K. In some embodiments, X₃ is E, and X₇ is T. In some embodiments, X₃ is E, and X₈ is F. In some embodiments, X₃ is E, and X₁₁ is K. In some embodiments, X₃ is E, and X₁₉ is L. In some embodiments, X₃ is E, and X₂₁ is Y. In some embodiments, X₃ is E, and X₂₃ is S.

In some embodiments, X₃ is E, and X₅ taken together with X₈ is represented by Formula (I). In some embodiments, X₃ is E, and X₅ taken together with X₁₂ is represented by Formula (I). In some embodiments, X₃ is E, and X₈ taken together with X₁₂ is represented by Formula (I). In some embodiments, X₃ is E, and X₈ taken together with X₁₆ is represented by Formula (I). In some embodiments, X₃ is E, and X₁₂ taken together with X₁₆ is represented by Formula (I). In some embodiments, X₃ is E, and X₁₆ taken together with X₂₀ is represented by Formula (I).

In some embodiments, X₅ is A, and X₆ is K. In some embodiments, X₅ is A, and X₇ is T. In some embodiments, X₅ is A, and X₈ is F. In some embodiments, X₅ is A, and X₁₁ is K. In some embodiments, X₅ is A, and X₁₉ is L. In some embodiments, X₅ is A, and X₂₁ is Y. In some embodiments, X₅ is A, and X₂₃ is S.

In some embodiments, X₆ is K, and X₇ is T. In some embodiments, X₆ is K, and X₈ is F. In some embodiments, X₆ is K, and X₁₁ is K. In some embodiments, X₆ is K, and X₁₉ is L. In some embodiments, X₆ is K, and X₂₁ is Y. In some embodiments, X₆ is K, and X₂₃ is S.

In some embodiments, X₇ is T, and X₈ is F. In some embodiments, X₇ is T, and X₁₁ is K. In some embodiments, X₇ is T, and X₁₉ is L. In some embodiments, X₇ is T, and X₂₁ is Y. In some embodiments, X₇ is T, and X₂₃ is S.

In some embodiments, X₈ is F, and X₁₁ is K. In some embodiments, X₈ is F, and X₁₉ is L. In some embodiments, X₈ is F, and X₂₁ is Y. In some embodiments, X₈ is F, and X₂₃ is S.

In some embodiments, X₁₁ is K, and X₁₉ is L. In some embodiments, X₁₁ is K, and X₂₁ is Y. In some embodiments, X₁₁ is K, and X₂₃ is S.

In some embodiments, X₂₁ is Y, and X₅ taken together with X₈ is represented by Formula (I). In some embodiments, X₂₁ is Y, and X₅ taken together with X₂ is represented by Formula (I). In some embodiments, X₂₁ is Y, and X₈ taken together with X₁₂ is represented by Formula (I). In some embodiments, X₂₁ is Y, and X₈ taken together with X₁₆ is represented by Formula (I). In some embodiments, X₂₁ is Y, and X₂ taken together with X₁₆ is represented by Formula (I). In some embodiments, X₂₁ is Y, and X₁₆ taken together with X₂₀ is represented by Formula (I).

In some embodiments, X₂₃ is S, and X₅ taken together with X₈ is represented by Formula (I). In some embodiments, X₂₃ is S, and X₅ taken together with Xu is represented by Formula (I). In some embodiments, X₂₃ is S, and X₈ taken together with Xu is represented by Formula (I). In some embodiments, X₂₃ is S, and X₈ taken together with X₁₆ is represented by Formula (I). In some embodiments, X₂₃ is S, and Xu taken together with X₁₆ is represented by Formula (I). In some embodiments, X₂₃ is S, and X₁₆ taken together with X₂₀ is represented by Formula (I).

In some embodiments, X₅ and X₈ are taken together to form a group represented by Formula (I); X₅ and X₂ are taken together to form a group represented by Formula (I); X₈ and X₁₂ are taken together to form a group represented by Formula (I); X₈ and X₁₆ are taken together to form a group represented by Formula (I), X₁₂ and X₁₆ are taken together to form a group represented by Formula (I); or X₁₆ and X₂₀ are taken together to form a group represented by Formula (I).

In some embodiments, the group

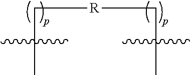

of Formula (I) is selected from the group consisting of

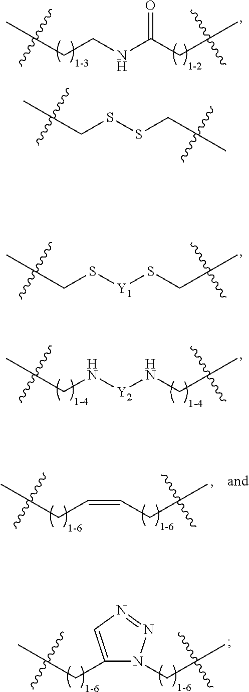

wherein:
Y₁ is aryl, -aryl-aryl-, perfluoroaryl, -perfluoroaryl-perfluoroaryl-, C₁₋₆ alkyl, or —CH₂—C(O)—CH₂—; and
Y₂ is aryl or —C(O)—Y₁—C(O)—.

In some embodiments, the group

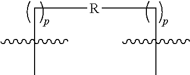

### of Formula (I) is

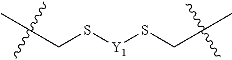

and Y₁ is perfluoroaryl or -perfluoroaryl-perfluoroaryl-.

In some embodiments, Y₁ is

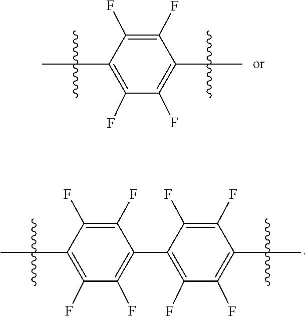 [SMILES: *c1c(F)c(F)c(*)c(F)c1F ; *c1c(F)c(F)c(-c2c(F)c(F)c(*)c(F)c2F)c(F)c1F]

In some embodiments, a peptide of the disclosure comprises an amino acid sequence selected from the group consisting of amino acid sequences represented by the consensus sequence of SEQ ID NO: 134:

X₀X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄;
wherein:
X₀ is QST, AQMYPLQEG, AQMYPLQEGG, or absent;
X₁ is I or absent;
X₂ is E or absent;
X₃ is E or absent;
X₄ is Q or absent;
X₅ is A, K, or 2-amino isobutyric acid (Aib), absent, or X₅ taken together with X₈ or X₁₂ is represented by Formula (I):

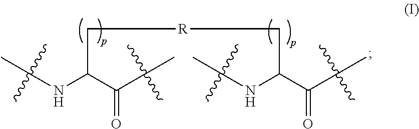

X₆ is K, K*, A, S, Q, H, T, or absent;
X₇ is T, L, D, Y, A, Aib, absent or is represented by Formula (II):

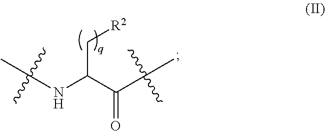

X₈ is F, S, T, H, N, D, E, A, Aib, absent, or X₈ taken together with X₅, X₁₂, or X₁₆ is represented by Formula (I);
X₉ is L, E, A, or absent;
X₁₀ is D, E, A, cysteic acid (Cya), or absent;
X₁₁ is K, Q, W, A, absent, or is represented by Formula (II):
X₁₂ is F, K, A. Aib, absent, or X₁₂ taken together with X₅, X₈, or X₁₆ is represented by Formula (I);
X₁₃ is N, K, H, D, A, or absent;
X₁₄ is H, E, V, A, Aib, 4-aminopiperidine-4-carboxylic acid (Pip), 4-fluorophenylalanine (Ff), or absent;
X₁₅ is E, A, or absent;
X₁₆ is A, E, Aib, Pip, absent, or X₁₆ taken together with X₅, X₁₂, or X₂₀ is represented by Formula (I);
X₁₇ is E, A, or absent;
X₁₈ is D, A, or absent;
X₁₉ is L, K, A, Aib, omithine (Orn), Pip or absent;
X₂₀ is F, K, A. Aib, absent, or X₂₀ taken together with X₁₆ is represented by Formula (I);
X₂₁ is Y, A, absent, or is represented by Formula (II);
X₂₂ is Q, K, or absent;
X₂₃ is S, Aib or absent;
X₂₄ is S, SLAS, SGGLGKGDFR, SpLGKGDFR, SkLGKGDFR, SGLGKGDFR, SGLGKGCyaFR, or absent;

wherein when any of X₁ through X₁₂ are absent, all residues prior to the absent residue are also absent;

wherein when any of X₁₃ through X₂₃ are absent, all residues subsequent to the absent residue are also absent;

wherein at least 12 consecutive residues are not absent;

wherein K* represents a lysine residue covalently bound at its ζ-nitrogen with C(O)(CH₂)ₘCH₃;

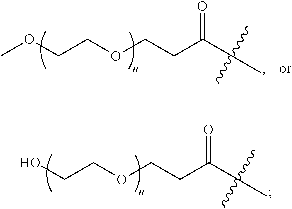

wherein the N-terminal amino group of the peptide is optionally substituted with C(O)(CH₂)ₘCH₃,

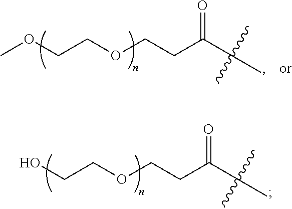

wherein the C-terminus of the peptide comprises a C-terminal acid group or a C-terminal amide group;

wherein R¹ is —NHC(O)—, —S—S—, —S-(aryl)-S—, —S-(aryl)-(aryl)-S—, —S-(perfluoroaryl)-S—, —S-(perfluoroaryl)-(perfluoroaryl)-S— —S—(C₁₋₆ alkyl)-S—, —S—CH₂—C(O)—CH₂—S—, —NH-(aryl)-NH—, —NHC(O)-(aryl)-C(O)NH—, —NHC(O)-(aryl)-(aryl)-C(O)NH—, —NHC(O)-(perfluoroaryl)-C(O)NH—, —NHC(O)-(perfluoroaryl)-(perfluoroaryl)-C(O)NH—, —NHC(O)—(C₁₋₆ alkyl)-C(O)NH—, —NHC(O)—CH₂—C(O)—CH₂—C(O)NH—, C₂₋₆ alkenyl, or heteroaryl;

wherein R² is aryl, heteroaryl, or C₃₋₈ cycloalkyl, each of which is optionally substituted with 1, 2, or substituents independently selected from the group consisting of C₁₋₆ alkyl, aryl, nitro, halo, cyano, amino, hydroxy, and C₁₋₃ alkylamino;

m is for each occurrence independently 0-16;

n is for each occurrence independently 4-1000; and

p is for each occurrence independently 1-6;

q is 1-3.

In some embodiments, X₀ is QST. In some embodiments, X₀ is AQMYPLQEG. In some embodiments, X₀ is absent.

In some embodiments, X₁ is I. In some embodiments, X₁ is absent.

In some embodiments, X₂ is E. In some embodiments, X₂ is absent.

In some embodiments, X₃ is E. In some embodiments, X₃ is absent.

In some embodiments, X₄ is Q. In some embodiments, X₄ is absent.

In some embodiments, X₅ is A. In some embodiments, X₅ is K. In some embodiments, X₅ is Aib. In some embodiments, X₅ is absent. In some embodiments, X₅ taken together with X₈ is represented by Formula (I). In some embodiments, X₅ taken together with X₁₂ is represented by Formula (I).

In some embodiments, X₆ is K. In some embodiments, X₁ is K*. In some embodiments, X₆ is A. In some embodiments, X₆ is S. In some embodiments, X₆ is Q. In some embodiments, X₆ is H. In some embodiments, X₆ is T. In some embodiments, X₆ is absent.

In some embodiments, X₇ is T. In some embodiments, X₇ is L. In some embodiments, X₇ is D. In some embodiments, X₇ is Y. In some embodiments, X₇ is A. In some embodiments, X₇ is Aib. In some embodiments, X₇ is absent. In some embodiments, X₇ is represented by Formula (II).

In some embodiments, X₈ is F. In some embodiments, X₈ is S. In some embodiments, X₈ is T. In some embodiments, X₈ is H. In some embodiments, X₈ is N. In some embodiments, X₈ is D. In some embodiments, X₈ is E. In some embodiments, X₈ is A. In some embodiments, X₈ is Aib. In some embodiments, X₈ is absent. In some embodiments, X₈ taken together with X₅ is represented by Formula (I). In some embodiments, X₈ taken together with Xu is represented by Formula (I). In some embodiments, X₈ taken together with X₁₆ is represented by Formula (I).

In some embodiments, X₉ is L. In some embodiments, X₉ is E. In some embodiments, X₉ is A. In some embodiments, X₉ is absent.

In some embodiments, X₁₀ is D. In some embodiments, X₁₀ is E. In some embodiments, X₁₀ is A. In some embodiments, X₁₀ is Cya. In some embodiments, X₁₀ is absent.

In some embodiments, X₁₁ is K. In some embodiments, X₁₁ is Q. In some embodiments, X₁₁ is W. In some embodiments, X₁₁ is A. In some embodiments, X₁₁ is absent. In some embodiments, X₁₁ is represented by Formula (II).

In some embodiments, X₁₂ is F. In some embodiments, X₁₂ is K. In some embodiments, X₁₂ is A. In some embodiments, X₁₂ is Aib. In some embodiments, Xu is absent. In some embodiments, X₁₂ taken together with X₈ is represented by Formula (I). In some embodiments, X₂ taken together with X₈ is represented by Formula (I). In some embodiments, X₁₂ taken together with X₁₆ is represented by Formula (I).

In some embodiments, X₁₂ is N. In some embodiments, X₁₃ is K. In some embodiments, X₁₃ is H. In some embodiments, X₁₃ is D. In some embodiments, X₁₃ is A. In some embodiments, X₁₃ is absent.

In some embodiments, X₁₄ is H. In some embodiments, X₁₄ is E. In some embodiments, X₁₄ is V. In some embodiments, X₁₄ is A. In some embodiments, X₁₄ is Aib. In some embodiments, X₁₄ is Pip. In some embodiments, X₁₄ is Ff. In some embodiments, X₁₄ is absent.

In some embodiments, X₁₅ is E. In some embodiments, X₁₅ is A. In some embodiments, X₁₅ is absent.

In some embodiments, X₁₆ is A. In some embodiments, X₁₆ is E. In some embodiments, X₁₆ is Aib. In some embodiments, X₁₆ is Pip. In some embodiments, X₁₆ is absent. In some embodiments, X₁₆ taken together with X₈ is represented by Formula (I). In some embodiments, X₁₆ taken together with X₁₂ is represented by Formula (I). In some embodiments, X₁₆ taken together with X₂₀ is represented by Formula (I).

In some embodiments, X₁₇ is E. In some embodiments, X₁₇ is A. In some embodiments, X₁₇ is absent.

In some embodiments, X₁₈ is D. In some embodiments, X₁₈ is A. In some embodiments, X₁₈ is absent.

In some embodiments, X₁₉ is L. In some embodiments, X₁₉ is K. In some embodiments, X₁₉ is A. In some embodiments, X₁₉ is Aib. In some embodiments, X₁₉ is Om. In some embodiments, X₁₉ is Pip. In some embodiments, X₁₉ is absent.

In some embodiments, X₂₀ is F. In some embodiments, X₂₀ is K. In some embodiments, X₂₀ is A. In some embodiments, X₂₀ is Aib. In some embodiments, X₂₀ is absent. In some embodiments, X₂₀ taken together with X₁₁ is represented by Formula (I).

In some embodiments, X₂₁ is Y. In some embodiments, X₂₁ is A. In some embodiments, X₂₁ is absent. In some embodiments, X₂₁ is represented by Formula (II).

In some embodiments, X₂₂ is Q. In some embodiments, X₂₂ is K. In some embodiments, X₂₂ is absent.

In some embodiments, X₂₃ is S. In some embodiments, X₂₃ is Aib. In some embodiments, X₂₃ is absent.

In some embodiments, X₂₄ is S. In some embodiments, X₂₄ is SLAS. In some embodiments, X₂₄ is SGGLGKGDFR. In some embodiments, X₂₄ is SpLGKGDFR. In some embodiments, X₂₄ is SkLGKGDFR. In some embodiments, X₂₄ is SGLGKGDFR. In some embodiments, X₂₄ is SGLGKGCyaFR. In some embodiments, X₂₄ is absent.

In some embodiments, X₂ is E, and X₆ is K. In some embodiments, X₂ is E, and X₁₄ is H. In some embodiments, X₂ is E, and X₁₇ is E. In some embodiments, X₂ is E, and X₂₁ is Y. In some embodiments, X₂ is E, and X₂₂ is Q.

In some embodiments, X₇ is T, and X₉ is L. In some embodiments, X₇ is T, and X₁₁ is K. In some embodiments, X₇ is T, and X₁₁ is W. In some embodiments, X₇ is T, and X₁₁ is represented by Formula (II). In some embodiments, X₇ is T, and X₁₇ is E. In some embodiments, X₇ is T, and X₁₈ is D. In some embodiments, X₇ is T, and X₁₉ is L. In some embodiments, X₇ is T, and X₂₁ is Y.

In some embodiments, X₈ is F, and X₉ is L. In some embodiments, X₈ is F, and X₁₁ is K. In some embodiments, X₈ is F, and X₁₁ is W. In some embodiments, X₈ is F, and X₁ is represented by Formula (II). In some embodiments, X₈ is F, and X₁₇ is E. In some embodiments, X₈ is F, and X₁₈ is D. In some embodiments, X₈ is F, and X₁₉ is L. In some embodiments, X₈ is F, and X₂₁ is Y.

In some embodiments, X₉ is L, and X₁₁ is K. In some embodiments, X₉ is L, and X₁₁ is W. In some embodiments, X₉ is L, and X₁₁ is represented by Formula (II). In some embodiments, X₉ is L, and X₁₇ is E. In some embodiments, X₉ is L, and X₁₈ is D. In some embodiments, X₉ is L, and X₁₉ is L. In some embodiments, X₉ is L, and X₂₁ is Y.

In some embodiments, X₁₁ is W, and X₁₇ is E. In some embodiments, X₁₁ is W, and X₁₈ is D. In some embodiments, X₁₁ is W, and X₁₉ is L. In some embodiments, X₁₁ is W, and X₂₁ is Y.

In some embodiments, X₁₁ is represented by Formula (II), and X₁₇ is E. In some embodiments, X₁₁ is represented by Formula (II), and X₁₈ is D. In some embodiments, X₁₁ is represented by Formula (II), and X₁₉ is L. In some embodiments, X₁₁ is represented by Formula (II), and X₂₁ is Y.

In some embodiments, X₁₇ is E, and X₁₈ is D. In some embodiments, X₁₇ is E, and X₁₉ is L. In some embodiments, X₁₇ is E, and X₂₁ is Y.

In some embodiments, X₇ is T, L, D, Y, A, or Aib;

X₈ is F, S, T, H, N, D, E, A, Aib, or X₈ taken together with X₅, X₁₂, or X₁₆ is represented by Formula (I);

X₉ is L, E, or A;

X₁₀ is D, E, A, or Cya;

X₁₁ is K, Q, W, A, or is represented by Formula (II);

X₁₂ is F, K, A. Aib, or X₂ taken together with X₅, X₈, or X₁₆ is represented by Formula (I);

X₁₃ is N, K, H, D, or A;

X₁₄ is H, E, V, A, Aib, Pip, or Ff;

X₁₅ is E or A;

X₁₆ is A, E, Aib, Pip, or X₁₆ taken together with X₈, X₁₂, or X₂₀ is represented by Formula (I);

X₁₇ is E or A;

X₁₈ is D or A;

wherein when X₁, X₂, X₃, X₄, X₅, or X₆ are absent, all previous residues are also absent; and wherein when X₁₉, X₂₀, X₂₁, X₂₂, or X₂₃ are absent, all subsequent residues are also absent.

In some embodiments, at least 13 consecutive residues are not absent. In some embodiments, at least 14 consecutive residues are not absent. In some embodiments, at least consecutive residues are not absent. In some embodiments, X₇, X₈, X₉, X₁₀, X₁₁, X₁₂, X₁₃, X₁₄, X₁₅, X₁₆, X₁₇, X₁₈, X₁₉, X₂₀, and X₂₁ are not absent.

In some embodiments, the structure of Formula (II) is selected from the group consisting of:

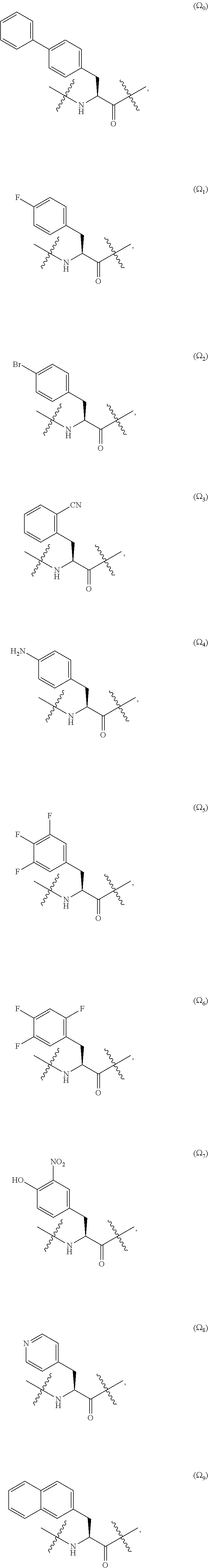

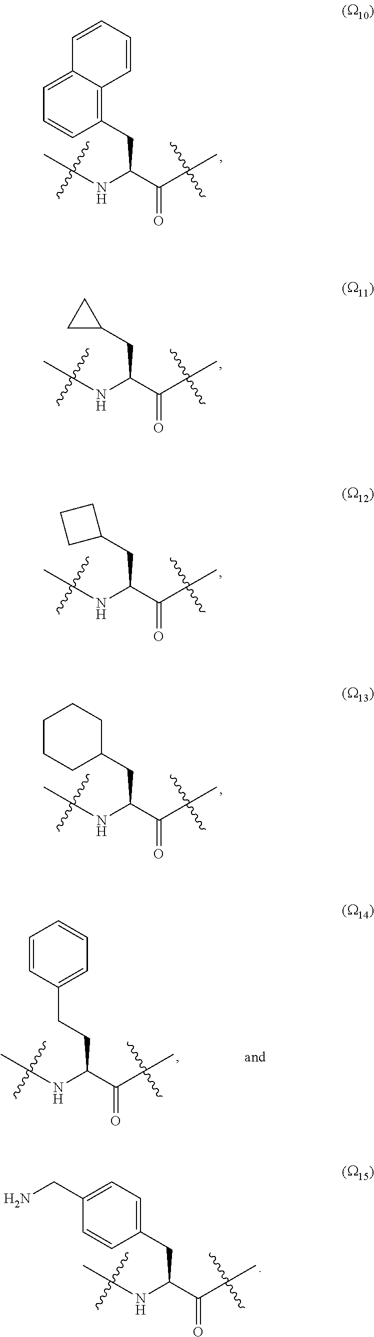 [SMILES: *N[C@@H](Cc1cccc2ccccc12)C(*)=O ; *N[C@@H](CC1CC1)C(*)=O ; *N[C@@H](CC1CCC1)C(*)=O ; *N[C@@H](CC1CCCCC1)C(*)=O ; *N[C@@H](CCc1ccccc1)C(*)=O ; *N[C@@H](Cc1ccc(CN)cc1)C(*)=O]

In some embodiments, the peptide is selected from the peptides described in Tables 1 and 2, below. The peptides of the disclosure, or their pharmaceutically acceptable salts may contain one or more asymmetric centers and may thus give rise to enantiomers, diastereomers, and other stereoisomeric forms that may be defined, in terms of absolute stereochemistry, as (R) or (S) or, as (D) or (L) for amino acids. The present disclosure is meant to include all such possible isomers, as well as their racemic and optically pure forms. Optically active (+) and (−), (R) and (S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the peptides described herein contain olefinic double bonds or other centers of geometric asymmetry, and unless specified otherwise, it is intended that the peptides include both E and Z geometric isomers. Likewise, all tautomeric forms are also intended to be included. (D)-amino acids (also referred to as D-amino acids) are referred to herein in lower case letters (e.g. D-valine is referred to as “v”), while (L)-amino acids (also referred to herein as L-amino acids) are referred to in upper case letters (e.g. L-valine or valine is referred to as “V”). Glycine is non-chiral and is referred to as “G”.

TABLE 1

| | |
|---|---|
| SEQ ID NO: 3 | IEEQAKTFLDKFNHEAEDLFYQS |
| SEQ ID NO: 4 | IEEQAKTFLDKFNHEAEDLFYQ |
| SEQ ID NO: 5 | IEEQAKTFLDKFNHEAEDLFY |
| SEQ ID NO: 6 | IEEQAKTFLDKFNHEAEDLF |
| SEQ ID NO: 7 | IEEQAKTFLDKFNHEAEDL |
| SEQ ID NO: 8 | IEEQAKTFLDKFNHEAED |
| SEQ ID NO: 9 | EEQAKTFLDKFNHEAEDLFYQS |
| SEQ ID NO: 10 | EQAKTFLDKFNHEAEDLFYQS |
| SEQ ID NO: 11 | QAKTFLDKFNHEAEDLFYQS |
| SEQ ID NO: 12 | AKTFLDKFNHEAEDLFYQS |
| SEQ ID NO: 13 | KTFLDKFNHEAEDLFYQS |
| SEQ ID NO: 14 | TFLDKFNHEAEDLFYQS |
| SEQ ID NO: 15 | TFLDKFNHEAED |
| SEQ ID NO: 16 | STIEEQAKTFLDKFNHEAEDLFYQSSLAS |
| | WNYNTNITEENVQNMNNAGDKWSAFLKEQ |
| | STLAQMYPLQEIQ |
| SEQ ID NO: 17 | IEEQAKTFLDKFNHEAEDLFYQS |
| SEQ ID NO: 18 | IEEQAATFLDKFNHEAEDLFYQS |
| SEQ ID NO: 19 | IEEQASTFLDKFNHEAEDLFYQS |
| SEQ ID NO: 20 | IEEQAQTFLDKFNHEAEDLFYQS |
| SEQ ID NO: 21 | IEEQAKTFLDKFNHEAEDLFYQS |
| SEQ ID NO: 22 | IEEQATTFLDKFNHEAEDLFYQS |
| SEQ ID NO: 23 | IEEQAKTSLDKFNHEAEDLFYQS |
| SEQ ID NO: 24 | IEEQAKTTLDKFNHEAEDLFYQS |
| SEQ ID NO: 25 | IEEQAKTHLDKFNHEAEDLFYQS |
| SEQ ID NO: 26 | IEEQAKTNLDKFNHEAEDLFYQS |
| SEQ ID NO: 27 | IEEQAKTDLDKFNHEAEDLFYQS |
| SEQ ID NO: 28 | IEEQAKTFLDΩFNHEAEDLFYQS |
| SEQ ID NO: 29 | IEEQAibKTFLDKFNHEAEDLFYQS |
| SEQ ID NO: 30 | IEEQAKAibFLDKFNHEAEDLFYQS |
| SEQ ID NO: 31 | IEEQAKTFLDKAibNHEAEDLFYQS |
| SEQ ID NO: 32 | IEEQAKTFLDKFNHEAibEDLFYQS |
| SEQ ID NO: 33 | IEEQAKTFLDKFNHEAEDLAibYQS |
| SEQ ID NO: 40 | IEEQAKTFLDKFNHEAE |
| SEQ ID NO: 41 | TCLDKCNH |
| SEQ ID NO: 42 | STIEEQAKTFLDKFNHEAEDLFYQSSLAS |
| | WNYNTNITEENVQNMNNAGDKWSAFLKEQ |
| | STLAQMYPLQEIQNLTVKLQLQALQQN |
| SEQ ID NO: 45 | IEEQAKTFLDKFNHEAEDLFYQSSLAS |
| SEQ ID NO: 46 | QSTIEEQAKTFLDKF |
| SEQ ID NO: 47 | IEEQAKTFLDKFNHE |
| SEQ ID NO: 48 | QAKTFLDKFNHEAED |
| SEQ ID NO: 49 | TFLDKFNHEAEDLFY |
| SEQ ID NO: 50 | DKFNHEAEDLFYQSS |
| SEQ ID NO: 51 | NHEAEDLFYQSSLAS |
| SEQ ID NO: 52 | QSTIEEQAKTFLDKFNHEAEDLFYQSSLASW |
| | NYNTNITEENVQNMNNAGDKWSAFLKEQSTL |
| | IAQMYPLQEQNLTVKLQLQALQQNGS |
| SEQ ID NO: 53 | IEEQAKLFLDKFNHEAEDLFYQS |
| SEQ ID NO: 54 | IEEQAKDFLDKFNHEAEDLFYQS |
| SEQ ID NO: 55 | IEEQAKYFLDKFNHEAEDLFYQS |
| SEQ ID NO: 56 | IEEQAKTFLDWFNHEAEDLFYQS |
| SEQ ID NO: 57 | IEEQAKTFLEKFNHEAEDLFYQS |
| SEQ ID NO: 58 | IEEQKKTFEDKFNHEAEDLFYQS |
| SEQ ID NO: 59 | IEEQAKTELDKKNHEAEDLFYQS |
| SEQ ID NO: 60 | IEEQAKTFEDKFKHEAEDLFYQS |
| SEQ ID NO: 61 | IEEQAKTFLDKFKHEEEDKKYQS |
| SEQ ID NO: 62 | IEEQKKTEEDKKKHEEEDKKYQS |
| SEQ ID NO: 63 | IEEQAKTAibLDKFNHEAEDLFYQS |
| SEQ ID NO: 64 | IEEQAKTFLDKFNHEAEDAibFYQS |
| SEQ ID NO: 65 | IEEQAKTFLDKFNHEAEDLFYQAib |
| SEQ ID NO: 66 | FLDWFNHEAEDLFY |
| SEQ ID NO: 67 | LDWFNHEAEDLFY |
| SEQ ID NO: 68 | DWFNHEAEDLFY |
| SEQ ID NO: 69 | TFLDWFNHEAEDLF |
| SEQ ID NO: 70 | TFLDWFNHEAEDL |
| SEQ ID NO: 71 | TFLDWFNHEAEDLFY |
| SEQ ID NO: 72 | TFLCyaWFHEEAEDLFY |
| SEQ ID NO: 73 | TFLDWFNHEAIbEDLFY |
| SEQ ID NO: 74 | TFLCyaWFNHEAibEDLFY |
| SEQ ID NO: 75 | TFLDWFNVEAEDLFY |
| SEQ ID NO: 76 | TFLDWFNFfEAEDLFY |
| SEQ ID NO: 77 | TFLDWFDHEAEDLFY |
| SEQ ID NO: 78 | AFLDKFNHEAEDLFY |
| SEQ ID NO: 79 | TALDKFNHEAEDLFY |
| SEQ ID NO: 80 | TFADKFNHEAEDLFY |
| SEQ ID NO: 81 | TFLAKFNHEAEDLFY |
| SEQ ID NO: 82 | TFLDAFNHEAEDLFY |
| SEQ ID NO: 83 | TFLDKANHEAEDLFY |
| SEQ ID NO: 84 | TFLDKFAHEAEDLFY |
| SEQ ID NO: 85 | TFLDKFNAEAEDLFY |
| SEQ ID NO: 86 | TFLDKFNHAAEDLFY |
| SEQ ID NO: 87 | TFLDKFNHEAADLFY |
| SEQ ID NO: 88 | TFLDKFNHEAEALFY |
| SEQ ID NO: 89 | TFLDKFNHEAEDAFY |
| SEQ ID NO: 90 | TFLDKFNHEAEDLAY |
| SEQ ID NO: 91 | TFLDKFNHEAEDLFA |
| SEQ ID NO: 92 | EEQAKTFLDKFNHEAEDLFYQSSGGLGKGDFR |
| SEQ ID NO: 93 | EEQAKTFLDKFNHEAEDLFYQSSpLGKGDFR |
| SEQ ID NO: 94 | EEQAKTFLDKFNHEAEDLFYQSSkLGKGDFR |
| SEQ ID NO: 95 | EEQAKTFLCyaKFNHEAEDLFYQSSGLGKGDFR |
| SEQ ID NO: 96 | EEQAKTFLDKFNHEAEDLFYQSSGLGKGCyaFR |
| SEQ ID NO: 97 | EEQAKTFLDKFNFfEAEDLFYQSSGLGKGDFR |
| SEQ ID NO: 98 | EEQAKTFLDKFNVEAEDLFYQSSGLGKGDFR |
| SEQ ID NO: 99 | GLGKGDFR |
| SEQ ID NO: 100 | AQMYPLQEG |
| SEQ ID NO: 101 | AQMYPLQEGIEEQAKTFLDKFNHEAED |
| | LFYQSSGLGKGDFR |
| SEQ ID NO: 102 | AQMYPLQEGGIEEQAKTFLDKFNHEAEDLF |
| | YQSSGLGKGDFR |
| SEQ ID NO: 103 | AQMYPLQEGIEEQAKTFLDKFNHEAEDLFYQSS |
| SEQ ID NO: 104 | YFLDWFNHEAEDLFY |
| SEQ ID NO: 105 | TFLDWFNHEAEDKFY |
| SEQ ID NO: 106 | TFLDWFNAEAEDLFY |
| SEQ ID NO: 107 | TFLDWFNAibEAEDLFY |
| SEQ ID NO: 108 | TFLDWFNHEAEDOrFY |
| SEQ ID NO: 109 | TFLDWFNHEAEDPipFY |
| SEQ ID NO: 110 | TFLDWFNHEPipEDLFY |
| SEQ ID NO: 111 | TFLDWFNPipEAEDLFY |
| SEQ ID NO: 112 | TFLDWFNHEAEDLFYK |
| SEQ ID NO: 113 | SLDQINVTFLDLEYEMKKLEEAIKKLEESYI |
| | DLKELGSGSGSGSGSTFLDWFNHEAEDLFY |
| SEQ ID NO: 114 | SLDQINVTFLDLEYEMKKLEEAIKKLEESYI |
| | DLKELGSGRRARSGSTFLDWFNHEAEDLFY |
| SEQ ID NO: 115 | TFLDΩFNHEAEDLFY |
| SEQ ID NO: 116 | TFLDΩFNHEAEDLFY |
| SEQ ID NO: 117 | TFLDΩFNHEAEDLFY |
| SEQ ID NO: 118 | TFLDΩFNHEAEDLFY |
| SEQ ID NO: 119 | TFLDΩFNHEAEDLFY |
| SEQ ID NO: 120 | TFLDΩFNHEAEDLFY |
| SEQ ID NO: 121 | TFLDΩFNHEAEDLFY |
| SEQ ID NO: 122 | TFLDΩFNHEAEDLFY |
| SEQ ID NO: 123 | TFLDΩsFNHEAEDLFY |
| SEQ ID NO: 124 | TFLDΩFNHEAEDLFY |
| SEQ ID NO: 125 | TFLDΩwFNHEAEDLFY |
| SEQ ID NO: 126 | TFLDΩFNHEAEDLFY |
| SEQ ID NO: 127 | TFLDΩFNHEAEDLFY |
| SEQ ID NO: 128 | TFLDΩbFNHEAEDLFY |
| SEQ ID NO: 129 | TFLDΩwFNHEAEDLFY |
| SEQ ID NO: 130 | TFLDΩIsFNHEAEDLFY |
| SEQ ID NO: 131 | IEEQAKTFLDKFNHEAEDLFYQSS |
| SEQ ID NO: 132 | TFLDWFNHEAEDLFY |

In some embodiments, the peptides are optionally stapled peptides between two X residues as specified in Table 2 below.

TABLE 2

| SEQ ID NO: | IEEQXKTXLDKFNHEAEDLFYQS |
|---|---|
| 34 | |
| SEQ ID NO: | IEEQXKTFLDKXNHEAEDLFYQS |
| 35 | |
| SEQ ID NO: | IEEQAKTXLDKXNHEAEDLFYQS |
| 36 | |
| SEQ ID NO: | IEEQAKTXLDKFNHEXEDLFYQS |
| 37 | |
| SEQ ID NO: | IEEQAKTFLDKXNHEXEDLFYQS |
| 38 | |
| SEQ ID NO: | IEEQAKTFLDKFNHEXEDLXYQS |
| 39 | |

*the two amino acids designated X together form a group represented by Formula (I).

In some embodiments, the peptide specifically binds to a receptor binding domain (RBD) of the spike protein of a coronavirus. In some embodiments, the spike protein specifically binds to the human ACE2 receptor. In some embodiments, the RBD specifically binds to the human ACE2 receptor.

In some embodiments, the peptide specifically binds to the spike protein of a coronavirus selected from the group consisting of human coronavirus 229E (HCoV-229E), human coronavirus OC43 (HCoV-OC43), severe acute respiratory syndrome coronavirus (SARS-CoV), human coronavirus NL63 (HCoV-NL63), human coronavirus HKU1, Middle East respiratory syndrome-related coronavirus (MERS-CoV) and severe acute respiratory syndrome coronavirus 2 (SARS-CoV-2). In some embodiments, the coronavirus is SARS-CoV-2.

Also contemplated herein are compositions comprising one or more peptides of the disclosure and at least one pharmaceutically acceptable carrier.

Multiple peptides of the disclosure may be conjugated together to form multivalent CoV-2 spike binding peptides. Accordingly, also disclosed herein are multivalent CoV-2 spike binding peptides having a structure according to Formula (III):

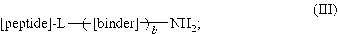

wherein L is an alkyl or heteroalkyl linker between 4 Å and 50 Å in length or L is absent; [binder] for each occurrence is independently selected from the group consisting of

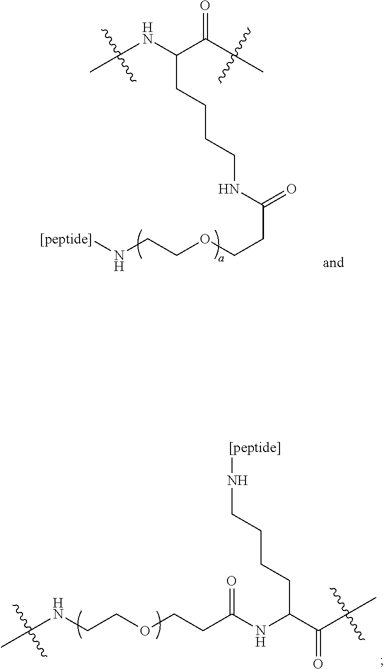

a is for each occurrence independently 1-12;
b is 1-9; and
[peptide], for each occurrence, independently is any one of the peptides disclosed herein.

In some embodiments, L is

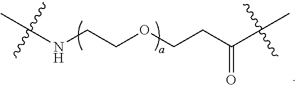

In some embodiments, the C-terminus of the peptide forms an amide bond with an amino group of L or [binder]. In some embodiments, the C-terminus of the peptide is covalently attached to [binder] via a 1,2,3-triazole.

In some embodiments, the CoV-2 spike binding peptide has a structure according to Formula (IV):

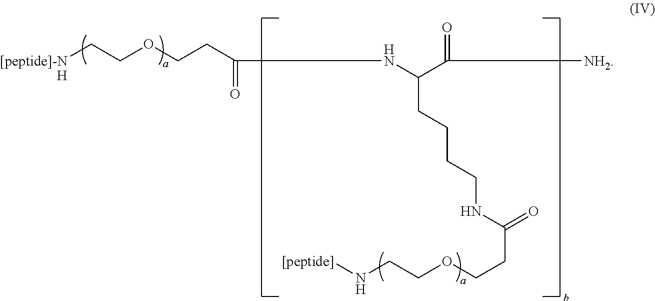

In some embodiments, the CoV-2 spike binding peptide has the following structure:

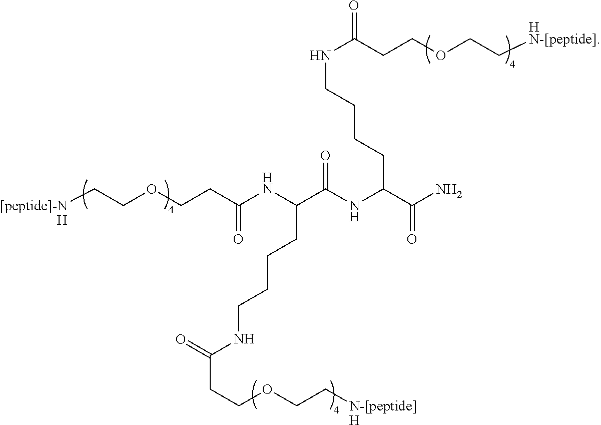

In some embodiments, the CoV-2 spike binding peptide has a structure according to Formula (V):

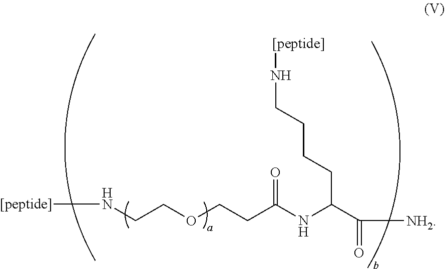

In some embodiments, the CoV-2 spike binding peptide has a structure selected from the group consisting of:

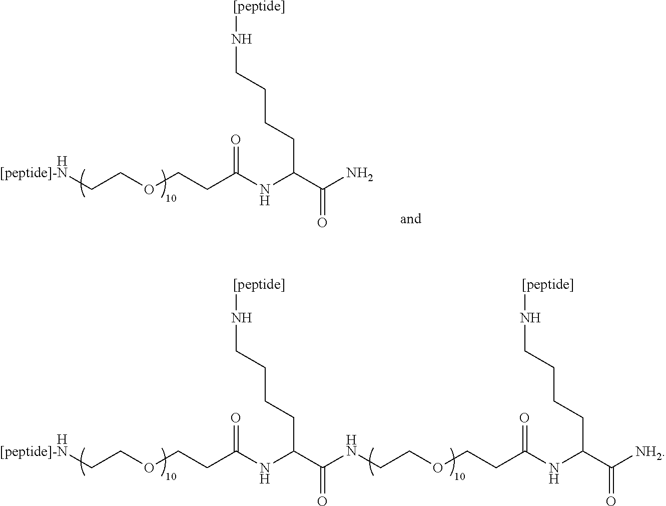

In some embodiment [peptide] is selected from the group consisting of SEQ ID NOS: 3-14, 16-33, 42, 45-98, 101-132.

In some embodiments, the multivalent Cov-2 spike binding protein specifically binds to a receptor binding domain (RBD) of the spike protein. In some embodiments, the spike protein specifically binds to human ACE2 receptor. In some embodiments, the RBD specifically binds to human ACE2 receptor. In some embodiments the multivalent Cov-2 spike binding protein binds the spike protein receptor binding domain of the coronavirus with a K_{D} of less than about 500 nM.

Also contemplated herein are compositions comprising one or more multivalent Cov-2 spike binding protein of the disclosure and at least one pharmaceutically acceptable carrier.

Also contemplated herein are methods of treatment comprising administering one or more of the peptides or multivalent CoV-2 spike binding peptides of the disclosure to a subject in heed thereof.

In another aspect, disclosed herein is a method of reducing probability of viral infection in a human subject, comprising administering a therapeutically effective amount of a peptide or multivalent CoV-2 spike binding peptide of the disclosure. In some embodiments, the viral infection is caused by a coronavirus. In some embodiments, the coronavirus is selected from the group consisting of HCoV-229E, HCoV-OC43, SARS-CoV, HCoV-NL63, human coronavirus HKU1, MERS-CoV and SARS-CoV-2. In some embodiments, the coronavirus is SARS-CoV-2.

In some embodiments, the administration of the peptide or multivalent CoV-2 spike binding peptide comprises intradermal, intramuscular, intraperitoneal, intravenous, subcutaneous, intranasal, epidural, sublingual, intracerebral, intravaginal, transdermal, rectal, by inhalation, or topical administration. In some embodiments, the human subject is at least 50 years of age.

In yet another aspect, disclosed herein is a method of detecting the presence of a viral infection in a human subject in need thereof, comprising (a) contacting a sample from the subject with a peptide or multivalent CoV-2 spike binding peptide of the disclosure, and (b) detecting specific binding between the composition and an antibody that specifically binds the infecting virus; wherein specific binding between the antibody and the composition indicates the presence of viral infection in the subject.

In some embodiments, the peptide or multivalent CoV-2 spike binding peptide is covalently bound to a solid phase substrate. In some embodiments, the detection of the detecting specific binding between the composition and the virus is performed using an immunoassay. In some embodiments, the immunoassay is an enzyme-linked immunosorbent assay (ELISA).

In some embodiments, the viral infection is caused by a coronavirus. In some embodiments, the coronavirus is selected from the group consisting of HCoV-229E, HCoV-OC43, SARS-CoV, HCoV-NL63, human coronavirus HKU1, MERS-CoV and SARS-CoV-2. In some embodiments, the coronavirus is SARS-CoV-2.

In some embodiments, the subject may not have a symptom, or may have a symptom associated with viral infection selected from the group consisting of fever, cough, shortness of breath, pain or pressure in the chest, confusion, bluish lips or face, pneumonia, bronchitis, runny nose, sneezing, chills, exacerbated asthma, acute respiratory distress syndrome (ARDS), RNAaemia, acute cardiac injury, shock, myalgia, fatigue, sputum production, rusty colored sputum, bloody sputum, swelling of lymph nodes, middle ear infection, joint pain, wheezing, headache, hemoptysis, diarrhea, dyspnea, redness, swelling or edema, pain, loss of function, organ dysfunction, multi-organ system failure, acute kidney injury, malnutrition, sepsis, hypotension, hypertension, hypothermia, hypoxemia, leukocytosis, leukopenia, lymphopenia, thrombocytopenia, nasal congestion, sore throat, unwillingness to drink, convulsions, ongoing vomiting, abdominal pain, secondary infection, cytokine release syndrome, and multi-organ failure.

In some embodiments, the human subject is at least 50 years of age.

Stapled peptides are short peptides, typically in an alpha-helical conformation, that are constrained by a synthetic brace (“staple”). The staple is formed by a covalent linkage between two amino acid side-chains, forming a peptide macrocycle. Peptides with multiple, tandem staples are sometimes referred to as stitched peptides. Peptide staples can be formed between residues of natural amino acids, or unnatural amino acids. Among other applications, peptide stapling is notably used to enhance the pharmacologic performance of peptides.

Introducing a synthetic brace (staple) helps to lock a peptide in a specific conformation, reducing conformational entropy. This approach can increase target affinity, increase cell penetration, and protect against proteolytic degradation. Various strategies have been employed for constraining α-helices, including non-covalent and covalent stabilization techniques; however, the all-hydrocarbon covalent link, termed a peptide staple, has been shown to have improved stability and cell penetrability, making this stabilization strategy particularly relevant for clinical applications.

Staples synthesized using ring-closing metathesis (RCM) are common (Journal of Med. Chem, 2014, 57(15): 6275-88.). This variation of olefin metathesis and its application to stapled peptides uses the Grubbs catalyst to cross-link O-allylserine residues via a covalent bond. The first synthesis of an all-hydrocarbon cross-link for peptide α-helix stabilization combined the principles of RCM with α,α-di-substitution of the amino acid chiral carbon and on-resin peptide synthesis. Later, it was demonstrated that stapling BH3 peptides enabled the synthetic peptides to retain their α-helical conformation, further demonstrating that these peptides were taken up by cancer cells and bound their physiologic BCL-2 family targets, which correlated with the induction of cell death. Further, it was discovered that the peptides side-stepped the membrane diffusion issue by crossing the membrane through active endosomal uptake, which deposited the peptides inside of the cell. Since this first proof of principle, peptide stapling technology has been applied to numerous peptide templates, allowing the study of many other PPIs using stapled peptides including cancer targets such as p53, MCL-1 BH3, and PUMA BH3, as well as other therapeutic targets ranging from infectious diseases to metabolism.

Scrambled peptides were used as a control to test binding of the peptides provided herein. Examples of scrambled peptides include EQAEKDNFHILKEYAQTDEFFSL (SEQ ID NO:43) and AIETAQSEHEKFQLNDYLDFKFE (SEQ ID NO:44).

As used herein the term “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are non-superimposable mirror images of one another.

As used herein the term “tautomer” refers to a proton shift from one atom of a molecule to another atom of the same molecule. The present invention includes tautomers of any said compounds.

Often crystallizations produce a solvate of the peptides of the invention. As used herein, the term “solvate” refers to an aggregate that comprises one or more peptides of the invention with one or more molecules of solvent. The solvent may be water, in which case the solvate may be a hydrate. Alternatively, the solvent may be an organic solvent. Thus, the peptides of the present invention may exist as anhydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

The term “epitope” as used herein refers to a distinct molecular surface of a protein. Typically, the epitope is a polypeptide with a finite sequence of 5-40 amino acids.

The terms “polypeptide,” “peptide,” and “protein” are used interchangeably herein to refer to an amino acid sequence comprising a polymer of amino acid residues. The terms apply to amino acid polymers in which one or more amino acid residues is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids, and isomers thereof. Naturally occurring amino acids are those encoded by the genetic code, as well as those amino acids that are later modified, e.g., hydroxyproline, carboxyglutamate, 0-phosphoserine, and isomers thereof. The term “amino acid analogs” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. The term “amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

The term “non-natural amino acid” as used herein refers to an amino acid that is different from the twenty naturally occurring amino acids (alanine, arginine, glycine, asparagine, aspartic acid, cysteine, glutamine, glutamic acid, serine, threonine, histidine, lysine, methionine, proline, valine, isoleucine, leucine, tyrosine, tryptophan, phenylalanine) in its side chain functionality, for example, α-amino isobutyric acid. The non-natural amino acid can be a close analog of one of the twenty natural amino acids, or it can introduce a completely new functionality and chemistry, as long as the hydrophobicity of the non-natural amino acid is either equivalent to or greater than that of the natural amino acid. The non-natural amino acid can either replace an existing amino acid in a protein (substitution), or be an addition to the wild type sequence (insertion). The incorporation of non-natural amino acids can be accomplished by known chemical methods including solid-phase peptide synthesis or native chemical ligation, staple chemistry or by biological methods.

The terms “specific binding,” “selective binding,” “selectively binds,” or “specifically binds” as used herein refer to capture agent binding to an epitope on a predetermined antigen. Typically, the peptide binds with an affinity (K_{D}) of approximately less than 500 nM, such as approximately less than 300 nM, 100 nM, 10 nM, 1 nM or even lower.

The term “K_{D}” as used herein refers to the dissociation equilibrium constant of a particular capture agent-antigen interaction. Typically, the peptides of the invention bind to a spike protein of a coronavirus with a dissociation equilibrium constant (K_{D}) of less than approximately, 500 nM, such as less than 300 nM, 100 nM, 10 nM, 1 nM or even lower, for example, as determined using bio-layer interferometry experiments, a K_{D} that is at least ten-fold lower, such as at least 100 fold lower, for instance at least 1000 fold lower, such as at least 10,000 fold lower, for instance at least 100,000 fold lower than its affinity for binding to a non-specific antigen (e.g., BSA, casein) other than the specific spike protein of a coronavirus or a closely-related protein.

A “pharmaceutical composition” refers to a formulation of a peptide of the invention and a medium generally accepted in the art for the delivery of the biologically active peptide to mammals, e.g., humans. Such a medium includes all pharmaceutically acceptable carriers, diluents or excipients therefor.

The pharmaceutical compositions disclosed herein can be administered by any therapeutically effective route. These routes include intradermal, intramuscular, intraperitoneal, intravenous, subcutaneous, intranasal, epidural, sublingual, intracerebral, intravaginal, transdermal, rectal, by inhalation, or topical administration.

In some embodiments, topical formulations described herein are for applications such as mucosal surface or keratinized skin surface of a mammalian subject, such as a human subject. By mucosal surface is meant a location of a subject that includes a mucosal membrane, such as the inside of the mouth, in the inside of the nose, etc. By keratinized skin surface is meant a skin location of a subject, i.e., a location of the external covering or integument of an animal body. Because the topical formulations of the invention are formulated for delivery to topical location, they are formulated so as to be physiologically compatible with the topical location for which they are formulated. Accordingly, when contacted with the target keratinized skin surface for which they are formulated, the topical compositions do not cause substantial, if any, physiological responses (such as inflammation or irritation) that would render the use of the topical compositions unsuitable for topical application. Topical formulations of the invention include: (a) an amount of the actives (which may or may not be stabilized); and (b) a topical delivery vehicle.

In some embodiments, topical formulations described herein include, but are not limited to, compositions that are suitable for applications via one or more of oral, topical, implantation, ocular, aural, rectal, vaginal, etc., routes. In certain embodiments, the vehicle is formulated for application to a topical region or surface of a subject, such as a keratinized skin surface. The subject compositions may be formulated as stable solutions or suspensions of the components, e.g., in an aqueous solvent. Where desired, the components may be combined with one or more carrier materials to form a solution, suspension, gel, lotion, cream, ointment, aerosol spray, roll-on, foam products, mousses, powders, sticks, or the like, as desired. Of interest in certain embodiments are aqueous delivery vehicles, i.e. aqueous vehicles that include a certain amount of water. Examples of aqueous vehicles include hydrogel vehicles, sprays, serums, etc.

The topical composition may also contain other physiologically acceptable excipients or other minor additives, particularly associated with organoleptic properties, such as fragrances, dyes, buffers, cooling agents (e.g. menthol), coating materials or the like. The excipients and minor additives will be present in conventional amounts, e.g., ranging from about 0.001% to 5%, such as 0.001-2%, by weight, and in some instances not exceeding a total of 10% by weight.

Lotions (as well as other topical formulations) may include one or more of the following components: water, viscosity modifiers, humectants, vegetable oils and hydrogenated vegetable oils, emollients, conditioning agents, emulsifiers, glyceryl esters of fatty acids, silicone, c1-c30 monoesters and polyesters of sugar, conditioning agents, preservatives, depending on the topical formulation. Additional components include abrasives, absorbents, antimicrobial and antifungal agents, astringents, anti-acne agents, anti-wrinkle agents, anti-oxidants, antimicrobials, binders, biological actives, buffering actives, bulking actives, chelating agents, chemical additives, external analgesics, film former agents, opacifying agents, pH adjusters, reducing agents, colorants, fragrances, cosmetic soothing agents, tanning actives and accelerators, skin lightening/whitening agents, sunscreens, surfactants, skin conditioning agents and vitamins.

Topical formulations also include semi-solid delivery compositions, such as gels, creams and ointments. In some embodiments, such compositions are mixtures of (in addition to the active agent) water, water soluble polymers, preservatives, alcohols, polyvalent alcohols, emulsifying agents, wax, solvents, thickeners, plasticizers, pH regulators, water-retaining agents and the like. Furthermore, such compositions may also contain other physiologically acceptable excipients or other minor additives, such as fragrances, dyes, buffers, coating materials or the like.

Topical formulations also include topical patch formulations. In some embodiments, topical patch formulations include an active agent layer, a support and a release liner. The active agent layer may include physiologically acceptable excipients or other minor additives, such as fragrances, dyes, buffers, coating materials or the like. In some embodiments, the support is made of a flexible material which is capable of fitting in the movement of human body and includes, for example, plastic films, various non-woven fabrics, woven fabrics, spandex, and the like. Various inert coverings may be employed, which include the various materials which may find use in plasters, described below. Alternatively, non-woven or woven coverings may be employed, particularly elastomeric coverings, which allow for heat and vapor transport. These coverings allow for cooling of the pain site, which provides for greater comfort, while protecting the gel from mechanical removal. The release liner may be made of any convenient material, where representative release films include polyesters, such as PET or PP, and the like.

The term “Ig constant domain” refers to the constant domain of an immunoglobulin. In some embodiments, the constant region of the immunoglobulin is the heavy chain constant region. In some embodiments, the immunoglobulin is IgG1, IgG2, IgG3, IgG4, IgA, IgE, IgM or IgD.

The terms “treat,” “treating,” or “treatment” as used herein generally refer to preventing a condition or event, slowing the onset or rate of development of a condition or delaying the occurrence of an event, reducing the risk of developing a condition or experiencing an event, preventing or delaying the development of symptoms associated with a condition or event, reducing or ending symptoms associated with a condition or event, generating a complete or partial regression of a condition, lessening the severity of a condition or event, or some combination thereof.

An “effective amount” or “therapeutically effective amount” as used herein refers to an amount effective, at dosages and for periods of time necessary, to achieve a desired therapeutic result. A therapeutically effective amount of a disclosed capture agent may vary according to factors such as the disease state, age, sex, and weight of the individual, and the ability of the capture agent to elicit a desired response in the individual.

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homo polymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination. As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelli genetics, Mountain View, Calif., USA).

As used herein, “reduce probability of viral infection” means preventing a significant percentage of subjects from developing symptoms associated with viral infection, for example, preventing at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantially identical” of polynucleotide sequences means that a polynucleotide comprises a sequence that has between 50-100% sequence identity, at least 50% sequence identity, at least 60% sequence identity, at least 70%, more at least 80%, more at least 90%, and most at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, at least 55%, at least 60%, more at least 70%, 80%, 90% and most at least 95%.

The term “solid phase surface”, “solid phase support”, “solid phase substrate” or other grammatical equivalents herein means any material that is appropriate for or can be modified to be appropriate for the attachment of the peptides, proteins and compositions provided herein. In some embodiments, the solid phase substrate is capable of binding viral particles. Possible surfaces include, but are not limited to, glass and modified or functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. In some embodiments, the solid phase substrate includes magnetic beads and high throughput microtier plates.

The composition and geometry of the solid support varies with its use. In some embodiments, solid supports comprise flat surfaces, microspheres or beads. By “microspheres” or “beads” or “particles” or grammatical equivalents herein is meant small discrete particles. The composition of the beads will vary, depending on the class of bioactive agent and the method of synthesis. Suitable bead compositions include those used in peptide, nucleic acid and organic moiety synthesis, including, but not limited to, plastics, ceramics, glass, polystyrene, methylstyrene, acrylic polymers, paramagnetic materials, thoria sol, carbon graphited, titanium dioxide, latex or cross-linked dextrans such as Sepharose, cellulose, nylon, cross-linked micelles and teflon, as well as any other materials outlined herein for solid supports may all be used.

A biological sample for use in the methods provided herein may be selected from the group consisting of organs, tissue, bodily fluids, and cells. Where the biological sample is a bodily fluid, the fluid may be selected from the group consisting of blood, serum, plasma, urine, sputum, saliva, stool, spinal fluid, cerebral spinal fluid, lymph fluid, skin secretions, respiratory secretions, intestinal secretions, genitourinary tract secretions, tears, and milk. The organs include, e.g., the adrenal glands, bladder, bones, brain, breasts, cervix, esophagus, eyes, gall bladder, genitals, heart, kidneys, large intestine, liver, lungs, lymph nodes, ovaries, pancreas, pituitary7 gland, prostate, salivary glands, skeletal muscles, skin, small intestine, spinal cord, spleen, stomach, thymus gland, trachea, thyroid, testes, ureters, and urethra.

Tissues include, e.g., epithelial, connective, nervous, lung and muscle tissues.

As used herein “immunoassay” refers to a method of detecting a protein target using a specific binding agent such as an antibody or antibody fragment. In one embodiment, the specific binding agent comprises a detectable label, such as an isotope, fluorescent label or enzyme. In one embodiment, the immunoassay produces a detectable color change in the presence of a protein target. In one embodiment, the immunoassay is an Enzyme Linked Immuno-Sorbent Assay (ELISA).

Enzyme Linked Immuno-Sorbent Assay (ELISA) is an antigen antibody reaction. It is a common laboratory technique which is usually used to measure the concentration of antibodies or antigens in blood. Enzyme-linked immunosorbent assay (ELISA) utilizes an enzyme system to show specific combination of an antigen with its antibody.

### EXAMPLES

### Example 1

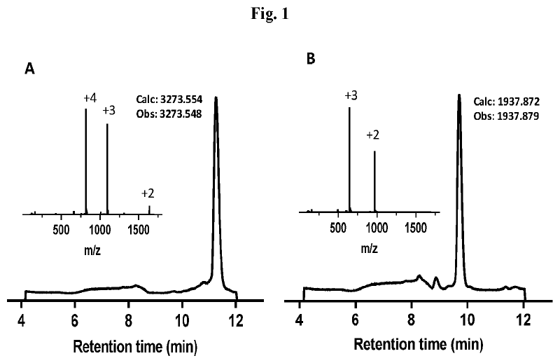

By leveraging expertise in molecular dynamic simulation and automated solid-phase peptide synthesis, we rapidly identified potent peptide binders to the SARS-CoV-2-RBD (FIG. 1), to the best of our knowledge, these are the first-in-class peptide binders to SARS-CoV-2-RBD.

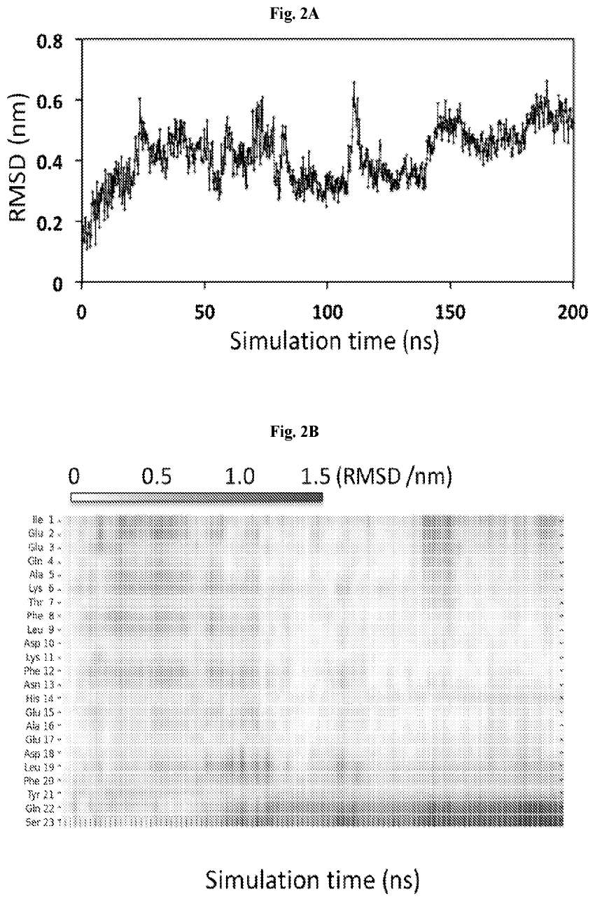

Computational modeling explained several important mutations from SARS-CoV-RBD to SARS-CoV-2-RBD directly involved in the binding to human ACE2 receptor. Specifically, a 200 ns molecular dynamics simulation of human ACE2-PD domain α-helix 1 and the SARS-CoV-2 S protein RBD domain complex revealed that the RMSD from residue 1 to 23 showed overall low fluctuation—an indicator of stable conformations (FIG. 2A, 2B). The model of binding interface between SARS-CoV-2-RBD and ACE2-h1 peptide after 200 ns simulation revealed that the main interaction forces included pi-pi stacking and H-bond from interface residues R403, K417, F456, N487, Y489 and Q493, among which K417, F456 and Q493 were the most prominent alternations from SARS-CoV-RBD to SARS-CoV-2-RBD based on the recent structural comparison (Renhong Yan et al., Science, 2020) and through MD simulation we validate that these SARS-CoV-2-RBD substitutions contribute directly to the binding for human ACE2 receptor (FIG. 2C).

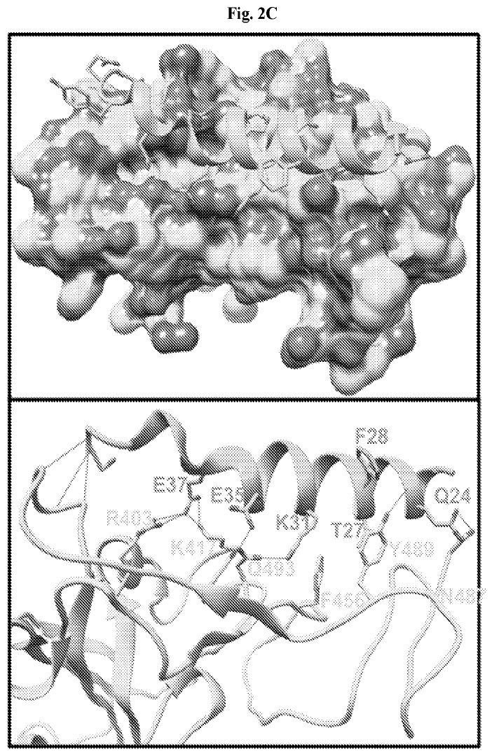

Using bio-layer interferometry experiments, we showed that a peptide extracted from the ACE2-PD al helix (IEEQAKTFLDKFNHEAEDLFYQS, SEQ ID NO: 3) could specifically associate insect-derived SARS-CoV-2-RBD with micromolar affinity (K_{D}=1.3 μM, FIG. 3A). However, although the middle residues of al helix showed relative importance by MD simulation, the peptide corresponding to the middle of al helix alone did not bind SARS-CoV-2-RBD (FIG. 3B), indicating this particular peptide-RBD interaction required a specific length.

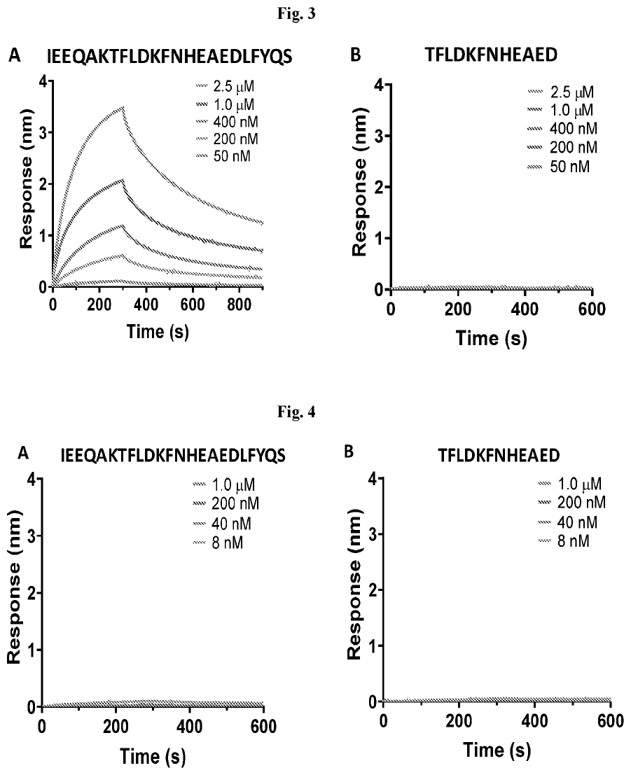

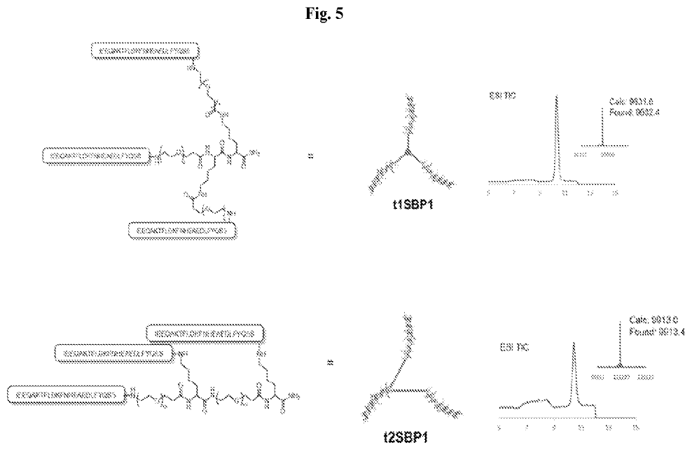

Using bio-layer interferometry experiments, we also confirmed that the ACE2-h1 peptide extracted from the ACE2-PD al helix was specific to SARS-CoV-2-RBD, as it did not bind unrelated human proteins (FIG. 4).

### Example 2

### Experimental Materials and Methods

### GPU-Accelerated Molecular Dynamic Simulation

The cryo-EM structure of ternary complex of SARS-CoV-2-RBD with ACE2-B⁰ATT (PDB: 6M17) was chosen as the initial structure, which was explicitly solvated in an 87 Å³ box, to perform a 200 ns molecular dynamical (MD) simulation using NAMD on MIT's supercomputing clusters (GPU node). The Amber force field was used to model the protein and peptide. The MD simulation system was equilibrated at 300 K for 2 ns. Periodic boundary conditions were used and long-range electrostatic interactions were calculated with particle mesh Ewald method, with non-bonded cutoff set to 12.0 Å. SHAKE algorithm was used to constrain bonds involving hydrogen atoms. Time step is 2 fs and the trajectories were recorded every 10 ps. After simulation production runs, trajectory files were loaded into the VMD software for further analysis.

### Automated Fast-Flow Peptide Synthesis

IEEQAKTFLDKFNHEAEDLFYQS (SEQ ID NO: 3) was synthesized at 90° C. on Rink Amide-ChemMatrix resin with HATU activation using a fully automatic flow-based peptide synthesizer. Amide bond formation was performed in 8 seconds, and Fmoc groups were removed in 8 seconds with 40% (v/v) piperidine in DMF. The overall synthesis cycle was completed in ˜120 seconds per amino acid incorporated. After completion of fast-flow synthesis, the resins were washed with DMF (3×) and then incubated with HATU-activated biotin-PEG4-propionic acid (CAS #721431-18-1) at room temperature for 1.0 h for biotinylation on the peptide N-terminus.

### Peptide Cleavage and Deprotection

After peptide synthesis, the peptidyl resin was rinsed with dichloromethane briefly and then dried in a vacuum chamber overnight. Next day, approximately 5 mL of cleavage solution (94% trifluoroacetic acid (TFA), 1% TIPS, 2.5% EDT, 2.5% water) was added into the syringe containing the resin. The syringe was kept at room temperature for 2 h before injecting the cleavage solution into a 50 mL conical tube. Dry-ice cold diethyl ether (˜50 mL) was added to the cleavage mixture and the precipitate was collected by centrifugation and triturated twice with cold diethyl ether (50 mL). The supernatant was discarded. Residual ether was allowed to evaporate and the peptide was dissolved in water with 0.1% TFA for solid-phase extraction.

### Solid-Phase Extraction (SPE)

After peptide cleavage, peptide precipitates were dissolved in water with 0.1% TFA. Agilent Mega BE C18 column (Part No: 12256130) was conditioned with 5 mL of 100% acetonitrile with 0.1% TFA, and then equilibrated with 15 mL of water with 0.1% TFA. Peptides were loaded onto the column for binding, followed by washing with 15 mL of water with 0.1% TFA, and finally, eluted with 5 mL of 30/70 water/acetonitrile (v/v) with 0.1% TFA.

Liquid Chromatography-Mass Spectrometry (LC-MS) Peptides were dissolved in water with 0.1% TFA followed by LC-MS analysis on an Agilent 6550 iFunnel ESI-Q-ToF instrument using an Agilent Jupiter C4 reverse-phase column (2.1 mm×150 mm, 5 μm particle size). Mobile phases were 0.10% formic acid in water (solvent A) and 0.1% formic acid in acetonitrile (solvent B). Linear gradients of 1 to 61% solvent B over 15 minutes (flow rate: 0.5 mL/min) were used to acquire LC-MS chromatograms.

### Kinetic Binding Assay Using Bio-Layer Interferometry (BLI)

A ForteBio Octet® RED96 Bio-Layer Interferometry system (Octet RED96, ForteBio, CA) was used to characterize the in vitro peptide-protein binding affinity at 30° C. and 1000 rpm. Briefly, streptavidin (SA) tips were dipped in 200 μL of biotinylated peptide solution (2.5 M in 1× kinetic buffer: 1×PBS with 0.1% BSA and 0.05% tween) for the loading step. The tips loaded with peptide were then sampled with commercially sourced SARS-CoV-2-RBD proteins (Sino Biological insect-derived RBD, CAT: 40592-V08B) or menin protein at various concentrations in 1× kinetic buffer to obtain the association curve. Peptide only was used as reference for background subtraction. After association, the tips were dipped back into 1× kinetic buffer to obtain the dissociation curve. The association and dissociation curves were fitted with ForteBio Biosystems using four experimental conditions (n=6, global fitting algorithm, binding model 1:1) to obtain the dissociation constants K_{D}.

1. A peptide that specifically binds to the spike protein of a coronavirus, wherein the peptide comprises an amino acid sequence selected from the group consisting of amino acid sequences represented by the consensus sequence of SEQ ID NO: 133:
X₁X₂X₃X₄X₅X₆X₇X₈LDX₁₁X₁₂NHEX₁₆EDX₁₉X₂₀X₂₁X₂₂X₂₃
(SEQ ID NO: 133):
wherein:
X₁ is I or absent;
X₂ is E or absent;
X₃ is E or absent;
X₄ is Q or absent;
X₅ is A, Aib, absent, or X₅ taken together with X₈ or X₁₂ is represented by Formula (I): 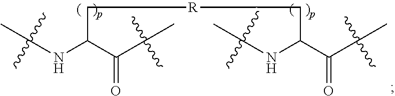
X₆ is K, K*, A, S, Q, H, T, or absent;
X₇ is T or Aib;
X₈ is F, S, T, H, N, D, or X₈ taken together with X₅, X₁₂, or X₁₆ is represented by Formula (I);
X₁₁ is K or Q;
X₁₂ is F, Aib, or X₁₂ taken together with X₅, X₈, or X₁₆ is represented by Formula (I);
X₁₆ is A, Aib, or X₁₆ taken together with X₈, X₁₂, or X₂₀ is represented by Formula (I);
X₁₉ is L or absent;
X₂₀ is F, Aib, absent, or X₂₀ taken together with X₁₆ is represented by Formula (I);
X₂₁ is Y or absent;
X₂₂ is Q or absent;
X₂₃ is S or absent;
wherein when X₂, X₃, X₄, X₅, or X₆ are absent, all previous residues are also absent;
wherein when X₁₉, X₂₀, X₂₁, or X₂₂ are absent, all subsequent residues are also absent;
wherein K* represents a lysine residue covalently bound at its ζ-nitrogen with C(O)(CH₂)ₘCH₃ or 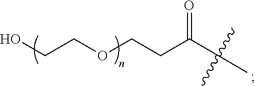
wherein the N-terminal amino group of the peptide is optionally substituted with C(O)(CH₂)ₘCH₃ or 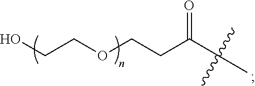
wherein the C-terminus of the peptide comprises a C-terminal acid group or a C-terminal amide group;
wherein R is —NHC(O)—, —S—S—, —S-(aryl)-S—, —S-(aryl)-(aryl)-S—, —S-(perfluoroaryl)-S—, —S-(perfluoroaryl)-(perfluoroaryl)-S— —S—(C₁₋₆ alkyl)-S—, —S—CH₂—C(O)—CH₂—S—, —NH-(aryl)-NH—, —NHC(O)-(aryl)-C(O)NH—, —NHC(O)-(aryl)-(aryl)-C(O)NH—, —NHC(O)-(perfluoroaryl)-C(O)NH—, —NHC(O)-(perfluoroaryl)-(perfluoroaryl)-C(O)NH—, —NHC(O)—(C₁₋₆ alkyl)-C(O)NH—, —NHC(O)—CH₂—C(O)—CH₂—C(O)NH—, C₂₋₆ alkenyl, or heteroaryl;
m is for each occurrence independently 0-16;
n is for each occurrence independently 4-1000; and
p is for each occurrence independently 1-6.

2. The peptide of claim 1, wherein m is 0.

3. The peptide of claim 1, wherein m is 14.

4. The peptide of claim 1, wherein 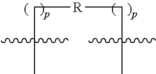
is selected from the group consisting of: 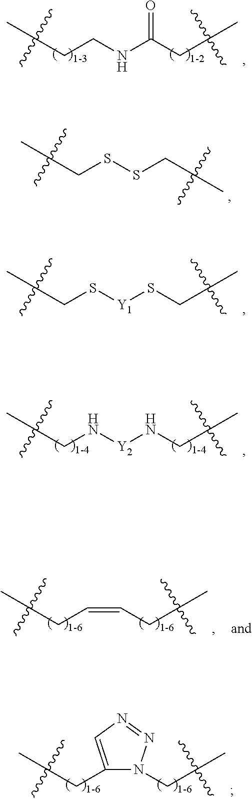
wherein:
Y₁ is aryl, -aryl-aryl-, perfluoroaryl, -perfluoroaryl-perfluoroaryl-, C₁₋₆ alkyl, or —CH₂—C(O)—CH₂—; and
Y₂ is aryl or —C(O)—Y₁—C(O)—.

5. The peptide of claim 4, wherein 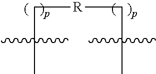
is 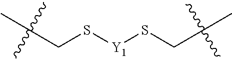
and Y₁ is perfluoroaryl or -perfluoroaryl-perfluoroaryl-.

6. The peptide of claim 5, wherein Y₁ is

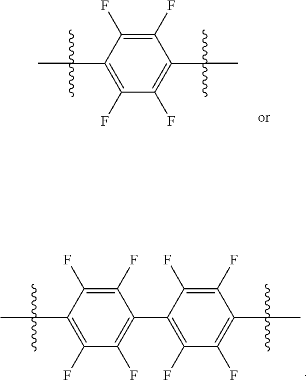 [SMILES: *c1c(F)c(F)c(*)c(F)c1F ; *c1c(F)c(F)c(-c2c(F)c(F)c(*)c(F)c2F)c(F)c1F]

7. The peptide of claim 1, wherein the one or more peptides comprise an amino acid sequence selected from the group consisting of:
(SEQ ID NO: 3)
IEEQAKTFLDKFNHEAEDLFYQS,
(SEQ ID NO: 4)
IEEQAKTFLDKFNHEAEDLFYQ,
(SEQ ID NO: 5)
IEEQAKTFLDKFNHEAEDLFY,
(SEQ ID NO: 6)
IEEQAKTFLDKFNHEAEDLF,
(SEQ ID NO: 7)
IEEQAKTFLDKFNHEAEDL,
(SEQ ID NO: 8)
IEEQAKTFLDKFNHEAED,
(SEQ ID NO: 9)
EEQAKTFLDKFNHEAEDLFYQS,
(SEQ ID NO: 10)
EQAKTFLDKFNHEAEDLFYQS,
(SEQ ID NO: 11)
QAKTFLDKFNHEAEDLFYQS,
(SEQ ID NO: 12)
AKTFLDKFNHEAEDLFYQS,
(SEQ ID NO: 13)
KTFLDKFNHEAEDLFYQS,
(SEQ ID NO: 14)
TFLDKFNHEAEDLFYQS,
(SEQ ID NO: 17)
IEEQAK*TFLDKFNHEAEDLFYQS,
(SEQ ID NO: 18)
IEEQAATFLDKFNHEAEDLFYQS,
(SEQ ID NO: 19)
IEEQASTFLDKFNHEAEDLFYQS,
(SEQ ID NO: 20)
IEEQAQTFLDKFNHEAEDLFYQS,
(SEQ ID NO: 21)
IEEQAHTFLDKFNHEAEDLFYQS,
(SEQ ID NO: 22)
IEEQATTFLDKFNHEAEDLFYQS,
(SEQ ID NO: 23)
IEEQAKTSLDKFNHEAEDLFYQS,
(SEQ ID NO: 24)
IEEQAKTTLDKFNHEAEDLFYQS,
(SEQ ID NO: 25)
IEEQAKTHLDKFNHEAEDLFYQS,
(SEQ ID NO: 26)
IEEQAKTNLDKFNHEAEDLFYQS,
(SEQ ID NO: 27)
IEEQAKTDLDKFNHEAEDLFYQS,
(SEQ ID NO: 28)
IEEQAKTFLDΩFNHEAEDLFYQS,
(SEQ ID NO: 29)
IEEQAibKTFLDKFNHEAEDLFYQS,
(SEQ ID NO: 30)
IEEQAKAibFLDKFNHEAEDLFYQS,
(SEQ ID NO: 31)
IEEQAKTFLDKAIbNHEAEDLFYQS,
(SEQ ID NO: 32)
IEEQAKTFLDKFNHEAibEDLFYQS,
(SEQ ID NO: 33)
IEEQAKTFLDKFNHEAEDLAibYQS,
(SEQ ID NO: 16)
STIEEQAKTFLDKFNHEAEDLFYQSS
LASWNYNTNITEENVQNMNNAGDKW
SAFLKEQSTLAQMYPLQEIQ,
and
(SEQ ID NO: 42)
STIEEQAKTFLDKFNHEAEDLFYQSS
LASWNYNTNITEENVQNMNNAGDKW
SAFLKEQSTLAQMYPLQEIQNLTVKL
QLQALQQN.

8. The peptide of claim 1, wherein one of the one or more peptides comprises the amino acid sequence of IEEQAKTFLDKFNHEAEDLFYQS (SEQ ID NO: 3).

9. The peptide of claim 1, wherein X₅ and X₈ are taken together to form a group represented by Formula (I); X₅ and X₁₂ are taken together to form a group represented by Formula (I); X₅ and X₁₂ are taken together to form a group represented by Formula (I); X₈ and X₁₆ are taken together to form a group represented by Formula (I), X₁₂ and X₁₆ are taken together to form a group represented by Formula (I); or X₁₆ and X₂₀ are taken together to form a group represented by Formula (I).

10. A peptide that specifically binds to the spike protein of a coronavirus, wherein the peptide comprises an amino acid sequence selected from the group consisting of amino acid sequences represented by the consensus sequence of SEQ ID NO: 134:
(SEQ ID NO: 134)
X₀X₁X₂X₃X₄X₅X₆X₇X₈X₉X₁₀X₁₁X₁₂X₁₃X₁₄X₁₅
X₁₆X₁₇X₁₈X₁₉X₂₀X₂₁X₂₂X₂₃X₂₄;
wherein:
X₀ is QST, AQMYPLQEG (SEQ ID NO: 135), AQMYPLQEGG (SEQ ID NO: 136), or absent;
X₁ is I or absent;
X₂ is E or absent;
X₃ is E or absent;
X₄ is Q or absent;
X₅ is A, K, 2-amino isobutyric acid (Aib), absent, or X₅ taken together with X₈ or X₁₂ is represented by Formula (I): 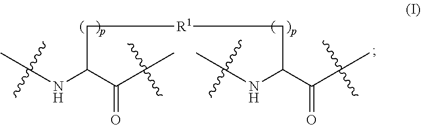
X₆ is K, K*, A, S, Q, H, T, or absent;
X₇ is T, L, D, Y, A, Aib, absent or is represented by Formula (II): 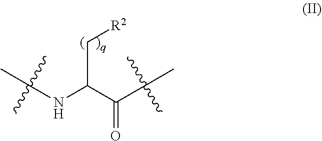
X₈ is F, S, T, H, N, D, E, A, Aib, absent, or X₈ taken together with X₅, X₁₂, or X₁₆ is represented by Formula (I);
X₉ is L, E, A, or absent;
X₁₀ is D, E, A, cysteic acid (Cya), or absent;
X₁₁ is K, Q, W, A, absent, or is represented by Formula (II);
X₁₂ is F, K, A, Aib, absent, or X₁₂ taken together with X₅, X₈, or X₁₆ is represented by Formula (I);
X₁₃ is N, K, H, D, A, or absent;
X₁₄ is H, E, V, A, Aib, 4-aminopiperidine-4-carboxylic acid (Pip), 4-fluorophenylalanine (Ff), or absent;
X₁₅ is E, A, or absent;
X₁₆ is A, E, Aib, Pip, absent, or X₁₆ taken together with X₈, X₁₂, or X₂₀ is represented by Formula (I);
X₁₇ is E, A, or absent;
X₁₈ is D, A, or absent;
X₁₉ is L, K, A, Aib, omithine (Orn), Pip or absent;
X₂₀ is F, K, A, Aib, absent, or X₂₀ taken together with X₁₆ is represented by Formula (I);
X₂₁ is Y, A, absent, or is represented by Formula (II);
X₂₂ is Q, K, or absent;
X₂₃ is S, Aib or absent;
X₂₄ is S, SLAS (SEQ ID NO: 137), SGGLGKGDFR (SEQ ID NO: 138), SpLGKGDFR (SEQ ID NO: 139), SkLGKGDFR (SEQ ID NO: 140), SGLGKGDFR (SEQ ID NO: 141), SGLGKGCyaFR (SEQ ID NO: 142), or absent;
wherein when any of X₁ through X₁₂ are absent, all residues prior to the absent residue are also absent;
wherein when any of X₁₃ through X₂₃ are absent, all residues subsequent to the absent residue are also absent;
wherein at least 12 consecutive residues are not absent;
wherein K* represents a lysine residue covalently bound at its ζ-nitrogen with C(O)(CH₂)ₘCH₃; 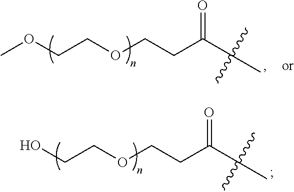
wherein the N-terminal amino group of the peptide is optionally substituted with C(O)(CH₂)ₘCH₃, 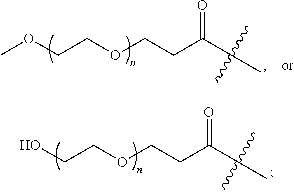
wherein the C-terminus of the peptide comprises a C-terminal acid group or a C-terminal amide group;
wherein R¹ is —NHC(O)—, —S—S—, —S-(aryl)-S—, —S-(aryl)-(aryl)-S—, —S-(perfluoroaryl)-S—, —S-(perfluoroaryl)-(perfluoroaryl)-S— —S—(C₁₋₆ alkyl)-S—, —S—CH₂—C(O)—CH₂—S—, —NH-(aryl)-NH—, —NHC(O)-(aryl)-C(O)NH—, —NHC(O)-(aryl)-(aryl)-C(O)NH—, —NHC(O)-(perfluoroaryl)-C(O)NH—, —NHC(O)-(perfluoroaryl)-(perfluoroaryl)-C(O)NH—, —NHC(O)—(C₁₋₆ alkyl)-C(O)NH—, —NHC(O)—CH₂—C(O)—CH₂—C(O)NH—, C₂₋₆ alkenyl, or heteroaryl;
wherein R² is aryl, heteroaryl, or C₃₋ₛ cycloalkyl, each of which is optionally substituted with 1, 2, or substituents independently selected from the group consisting of C₁₋₆ alkyl, aryl, nitro, halo, cyano, amino, hydroxy, and C₁₋₃ alkylamino;
m is for each occurrence independently 0-16;
n is for each occurrence independently 4-1000; and
p is for each occurrence independently 1-6;
q is 1-3.

11. The peptide of claim 10, wherein
X₇ is T, L, D, Y, A, or Aib;
X₈ is F, S, T, H, N, D, E, A, Aib, or X₈ taken together with X₅, X₁₂, or X₁₆ is represented by Formula (I);
X₉ is L, E, or A;
X₁₀ is D, E, A, or Cya;
X₁₁ is K, Q, W, A, or is represented by Formula (II): 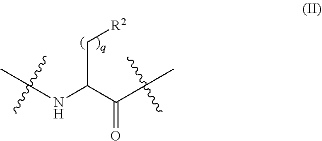
X₁₂ is F, K, A, Aib, or X₁₂ taken together with X₅, X₈, or X₁₆ is represented by Formula (I);
X₁₃ is N, K, H, D, or A;
X₁₄ is H, E, V, A, Aib, Pip, or Ff;
X₁₅ is E or A;
X₁₆ is A, E, Aib, Pip, or X₁₆ taken together with X₈, X₁₂, or X₂₀ is represented by Formula (I);
X₁₇ is E or A;
X₁₈ is D or A;
wherein when X₁, X₂, X₃, X₄, X₅, or X₆ are absent, all previous residues are also absent; and
wherein when X₁₉, X₂₀, X₂₁, X₂₂, or X₂₃ are absent, all subsequent residues are also absent.

12. The peptide of claim 10, wherein at least 15 consecutive residues are not absent.

13. The peptide of claim 10, wherein the structure of Formula (II) is selected from the group consisting of

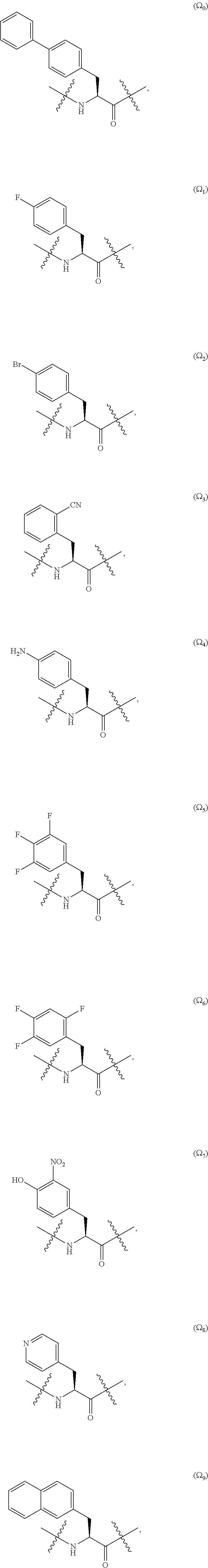

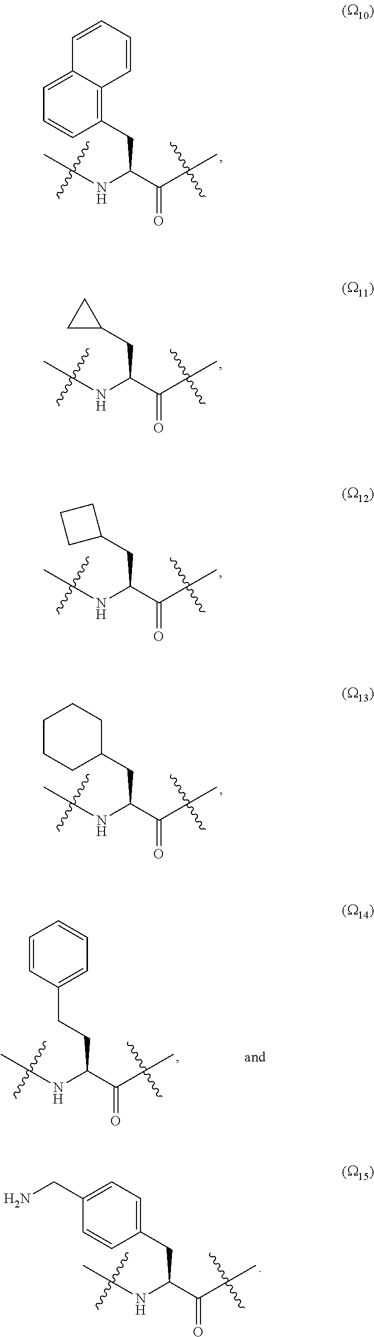 [SMILES: *N[C@@H](Cc1cccc2ccccc12)C(*)=O ; *N[C@@H](CC1CC1)C(*)=O ; *N[C@@H](CC1CCC1)C(*)=O ; *N[C@@H](CC1CCCCC1)C(*)=O ; *N[C@@H](CCc1ccccc1)C(*)=O ; *N[C@@H](Cc1ccc(CN)cc1)C(*)=O]

14. The peptide of claim 10, wherein the one or more peptides comprise a sequence selected from the group consisting of
(SEQ ID NO: 3)
IEEQAKTFLDKFNHEAEDLFYQS;
(SEQ ID NO: 45)
IEEQAKTFLDKFNHEAEDLFYQSSLAS;
(SEQ ID NO: 46)
QSTIEEQAKTFLDKF;
(SEQ ID NO: 47)
IEEQAKTFLDKFNHE;
(SEQ ID NO: 48)
QAKTFLDKFNHEAED;
(SEQ ID NO: 49)
TFLDKFNHEAEDLFY;
(SEQ ID NO: 50)
DKFNHEAEDLFYQSS;
(SEQ ID NO: 51)
NHEAEDLFYQSSLAS;
(SEQ ID NO: 52)
QSTIEEQAKTFLDKFNHEAEDLFYQSSLA
SWNYNTNITEENVQNMNNAGDKWSAFLKE
QSTLAQMYPLQEIQNLTVKLQLQALQQNGS;
(SEQ ID NO: 53)
IEEQAKLFLDKFNHEAEDLFYQS;
(SEQ ID NO: 54)
IEEQAKDFLDKFNHEAEDLFYQS;
(SEQ ID NO: 55)
IEEQAKYFLDKFNHEAEDLFYQS;
(SEQ ID NO: 56)
IEEQAKTFLDWFNHEAEDLFYQS;
(SEQ ID NO: 57)
IEEQAKTFLEKFNHEAEDLFYQS;
(SEQ ID NO: 58)
IEEQKKTFEDKFNHEAEDLFYQS;
(SEQ ID NO: 59)
IEEQAKTELDKKNHEAEDLFYQS;
(SEQ ID NO: 60)
IEEQAKTFEDKFKHEAEDLFYQS;
(SEQ ID NO: 61)
IEEQAKTFLDKFKHEEEDKKYQS;
(SEQ ID NO: 62)
IEEQKKTEEDKKKHEEEDKKYQS;
(SEQ ID NO: 29)
IEEQAibKTFLDKFNHEAEDLFYQS;
(SEQ ID NO: 32)
IEEQAKTFLDKFNHEAibEDLFYQS;
(SEQ ID NO: 63)
IEEQAKTAibLDKFNHEAEDLFYQS;
(SEQ ID NO: 31)
IEEQAKTFLDKAIbNHEAEDLFYQS;
(SEQ ID NO: 64)
IEEQAKTFLDKFNHEAEDAibFYQS;
(SEQ ID NOD 33)
IEEQAKTFLDKFNHEAEDLAibYQS;
(SEQ ID NO: 65)
IEEQAKTFLDKFNHEAEDLFYQAib;
(SEQ ID NO: 66)
FLDWFNHEAEDLFY;
(SEQ ID NO: 67)
LDWFNHEAEDLFY;
(SEQ ID NO: 68)
DWFNHEAEDLFY;
(SEQ ID NO: 69)
TFLDWFNHEAEDLF;
(SEQ ID NO: 70)
TFLDWFNHEAEDL;
(SEQ ID NO: 71)
TFLDWFNHEAEDLFY;
(SEQ ID NO: 72)
TFLCyaWFHEEAEDLFY;
(SEQ ID NO: 73)
TFLDWFNHEAibEDLFY;
(SEQ ID NO: 74)
TFLCydWFNHEAIbEDLFY;
(SEQ ID NO: 75)
TFLDWFNVEAEDLFY;
(SEQ ID NO: 76)
TFLDWFNFJEAEDLFY;
(SEQ ID NO: 77)
TFLDWFDHEAEDLFY;
(SEQ ID NO: 78)
AFLDKENHEAEDLFY;
(SEQ ID NO: 79)
TALDKFNHEAEDLFY;
(SEQ ID NO: 80)
TFADKENHEAEDLFY;
(SEQ ID NO: 81)
TFLAKFNHEAEDLFY;
(SEQ ID NO: 82)
TFLDAFNHEAEDLFY;
(SEQ ID NO: 83)
TFLDKANHEAEDLFY;
(SEQ ID NO: 84)
TFLDKFAHEAEDLFY;
(SEQ ID NO: 85)
TFLDKFNAEAEDLFY;
(SEQ ID NO: 86)
TFLDKFNHAAEDLFY;
(SEQ ID NO: 87)
TFLDKFNHEAADLFY;
(SEQ ID NO: 88)
TFLDKFNHEAEALFY;
(SEQ ID NO: 89)
TFLDKFNHEAEDAFY;
(SEQ ID NO: 90)
TFLDKFNHEAEDLAY;
(SEQ ID NO: 91)
TFLDKFNHEAEDLFA;
(SEQ ID NO: 92)
EEQAKTFLDKFNHEAEDLFYQSSGGLGKGDFR;
(SEQ ID NO: 93)
EEQAKTFLDKFNHEAEDLFYQSSpLGKGDFR;
(SEQ ID NO: 94)
EEQAKTFLDKFNHEAEDLFYQSSkLGKGDFR;
(SEQ ID NO: 95)
EEQAKTFLCyKFNHEAEDLFYQSSGLGKGDFR;
(SEQ ID NO: 96)
EEQAKTFLDKFNHEAEDLFYQSSGLGKGCyGFR;
(SEQ ID NO: 97)
EEQAKTFLDKFNFfEAEDLFYQSSGLGKGDFR;
(SEQ ID NO: 98)
EEQAKTFLDKFNVEAEDLFYQSSGLGKGDFR;
(SEQ ID NO: 101)
AQMYPLQEGIEEQAKTFLDKFNHEAEDLFYQSSGLGKGDFR;
(SEQ ID NO: 102)
AQMYPLQEGGIEEQAKTFLDKFNHEAEDLFYQSSGLGKGDFR;
(SEQ ID NO: 103)
AQMYPLQEGIEEQAKTFLDKFNHEAEDLFYQSS;
(SEQ ID NO: 104)
YFLDWFNHEAEDLFY;
(SEQ ID NO: 105)
TFLDWFNHEAEDKFY;
(SEQ ID NO: 106)
TFLDWFNAEAEDLFY;
(SEQ ID NO: 107)
TFLDWFNAIbEAEDLFY;
(SEQ ID NO: 108)
TFLDWFNHEAEDOrFY;
(SEQ ID NO: 109)
TFLDWFNHEAEDPipFY;
(SEQ ID NO: 110)
TFLDWFNHEPipEDLFY;
(SEQ ID NO: 111)
TFLDWFNPipEAEDLFY;
(SEQ ID NO: 112)
TFLDWFNHEAEDLFYK;
(SEQ ID NO: 113)
SLDQINVTFLDLEYEMKKLEEAIKKLEESYI;
DLKELGSGSGSGSGSTFLDWFNHEAEDLFY;
(SEQ ID NO: 114)
SLDQINVTFLDLEYEMKKLEEAIKKLEESYI
DLKELGSGRRARSGSTFLDWFNHEAEDLFY;
(SEQ ID NO: 115)
TFLDΩ₀FNHEAEDLFY;
(SEQ ID NO: 116)
TFLDΩ₁FNHEAEDLFY;
(SEQ ID NO: 117)
TFLDΩ₂FNHEAEDLFY;
(SEQ ID NO: 118)
TFLDΩ₃FNHEAEDLFY;
(SEQ ID NO: 119)
TFLDΩ₄FNHEAEDLFY;
(SEQ ID NO: 120)
TFLDΩ₅FNHEAEDLFY;
(SEQ ID NO: 121)
TFLDΩ₆FNHEAEDLFY;
(SEQ ID NO: 122)
TFLDΩ₇FNHEAEDLFY;
(SEQ ID NO: 123)
TFLDΩ₈FNHEAEDLFY;
(SEQ ID NO: 124)
TFLDΩ₉FNHEAEDLFY;
(SEQ ID NO: 125)
TFLDΩ₁₀FNHEAEDLFY;
(SEQ ID NO: 126)
TFLDΩ₁₁FNHEAEDLFY;
(SEQ ID NO: 127)
TFLDΩ₁₂FNHEAEDLFY;
(SEQ ID NO: 128)
TFLDΩ₁₃FNHEAEDLFY;
(SEQ ID NO: 129)
TFLDΩ1₄FNHEAEDLFY;
and
(SEQ ID NO: 130)
TFLDΩ₁₅FNHEAEDLFY.

15. A peptide comprising a sequence selected from the group consisting of
GLGKGDFR (SEQ ID NO: 99); and
AQMYPLQEG (SEQ ID NO: 100).

16. A multivalent CoV-2 spike binding peptide have a structure according to Formula (III): 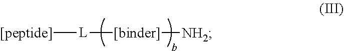
wherein L is an alkyl or heteroalkyl linker between 4 Å and 50 Å in length or L is absent;
[binder] for each occurrence is independently selected from the group consisting of 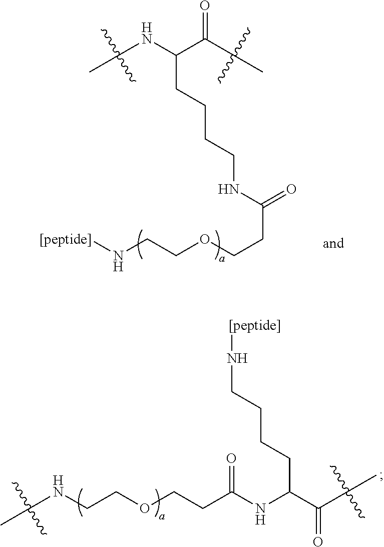
a is for each occurrence independently 1-12;
b is 1-9; and
[peptide], for each occurrence, independently comprises the formula:
(SEQ ID NO: 143)
X₀X₁X₂X₃X₄X₅X₆X₇X₈X₉X₁₀X₁₁X₁₂X₁₃X₁₄X₁₅
X₁₆X₁₇X₁₈X₁₉X₂₀X₂₁X₂₂X₂₃X₂₄;
wherein:
X₀ is QST, AQMYPLQEG (SEQ ID NO: 135), AQMYPLQEGG (SEQ ID NO: 136), or absent;
X₁ is I or absent;
X₂ is E or absent;
X₃ is E or absent;
X₄ is Q or absent;
X₅ is A, K, or 2-amino isobutyric acid (A/b), absent, or X₅ taken together with X₈ or X₁₂ is represented by Formula (I): 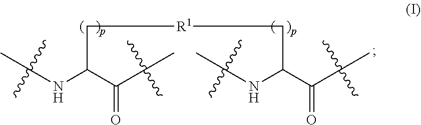
X₆ is K, K*, A, S, Q, H, T, or absent;
X₇ is T, L, D, Y, A, Aib, or absent;
X₈ is F, S, T, H, N, D, E, A, Aib, absent, or X₈ taken together with X₅, X₁₂, or X₁₆ is represented by Formula (I);
X₉ is L, E, A, or absent;
X₁₀ is D, E, A, cysteic acid (Cya), or absent;
X₁₁ is K, Q, W, A, absent, or is represented by Formula (II): 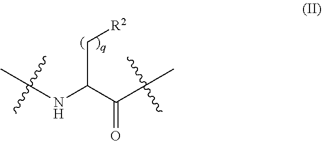
X₁₂ is F, K, A, Aib, absent, or X₁₂ taken together with X₅, X₈, or X₁₆ is represented by Formula (I);
X₁₃ is N, K, H, D, A, or absent;
X₁₄ is H, E, V, A, Aib, 4-aminopiperidine-4-carboxylic acid (Pip), 4-fluorophenylalanine (Ff), or absent;
X₁₅ is E, A, or absent;
X₁₆ is A, E, Aib, Pip, absent, or X₁₆ taken together with X₈, X₁₂, or X₂₀ is represented by Formula (I);
X₁₇ is E, A, or absent;
X₁₈ is D, A, or absent;
X₁₉ is L, K, A, Aib, omithine (Orn), Pip or absent;
X₂₀ is F, K, A, Aib, absent, or X₂₀ taken together with X₁₆ is represented by Formula (I);
X₂₁ is Y, A, or absent;
X₂₂ is Q, K, or absent;
X₂₃ is S, Aib or absent;
X₂₄ is S, SLAS (SEQ ID NO: 137), SGGLGKGDFR (SEQ ID NO: 138), SpLGKGDFR (SEQ ID NO: 139), SkLGKGDFR (SEQ ID NO: 140), SGLGKGDFR (SEQ ID NO: 141), SGLGKGCyaFR (SEQ ID NO: 142), or absent;
wherein when any of X₁ through X₁₂ are absent, all residues prior to the absent residue are also absent;
wherein when any of X₁₃ through X₂₃ are absent, all residues subsequent to the absent residue are also absent;
wherein at least 12 consecutive residues are not absent;
wherein K* represents a lysine residue covalently bound at its ζ-nitrogen with C(O)(CH₂)ₘCH₃ or 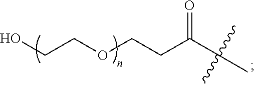
wherein the N-terminal amino group of the peptide is optionally substituted with C(O)(CH₂)ₘCH₃ or 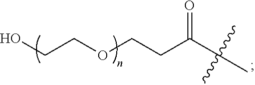
wherein the C-terminus of the peptide forms an amide bond with an amino group of L or [binder];
wherein R¹ is —NHC(O)—, —S—S—, —S-(aryl)-S—, —S-(aryl)-(aryl)-S—, —S-(perfluoroaryl)-S—, —S-(perfluoroaryl)-(perfluoroaryl)-S— —S—(C₁₋₆ alkyl)-S—, —S—CH₂—C(O)—CH₂—S—, —NH-(aryl)-NH—, —NHC(O)-(aryl)-C(O)NH—, —NHC(O)-(aryl)-(aryl)-C(O)NH—, —NHC(O)-(perfluoroaryl)-C(O)NH—, —NHC(O)-(perfluoroaryl)-(perfluoroaryl)-C(O)NH—, —NHC(O)—(C₁₋₆ alkyl)-C(O)NH—, —NHC(O)—CH₂—C(O)—CH₂—C(O)NH—, C₂₋₆ alkenyl, or heteroaryl;
wherein R² is aryl, heteroaryl, or C₃₋₈ cycloalkyl, each of which is optionally substituted with 1, 2, or substituents independently selected from the group consisting of C₁₋₆ alkyl, aryl, nitro, halo, cyano, amino, hydroxy, and C₁₋₃ alkylamino;
m is for each occurrence independently 0-16;
n is for each occurrence independently 4-1000; and
p is for each occurrence independently 1-6;
q is 1-3.

17. The multivalent CoV-2 spike binding peptide of claim 16, wherein L is

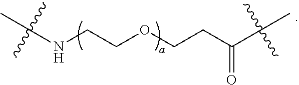

18. The multivalent CoV-2 spike binding peptide of claim 16, wherein the C-terminus of the peptide forms an amide bond with an amino group of L or [binder].

19. The multivalent CoV-2 spike binding peptide of claim 16, wherein the C-terminus of the peptide is covalently attached to [binder] via a 1,2,3-triazole.

20. The multivalent CoV-2 spike binding peptide of claim 16 having a structure according to Formula (IV): 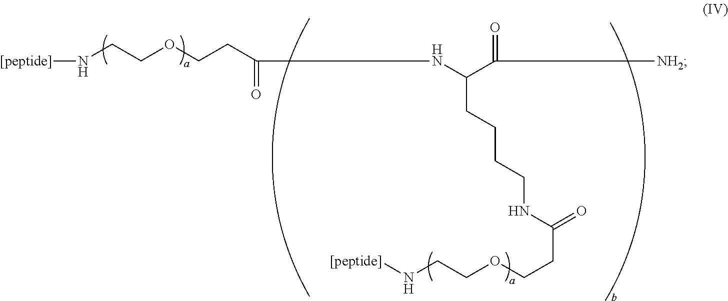
wherein, a, b, and [peptide] are as defined in claim 16.

21. The multivalent Cov-2 spike binding protein of claim 20 having the following structure:

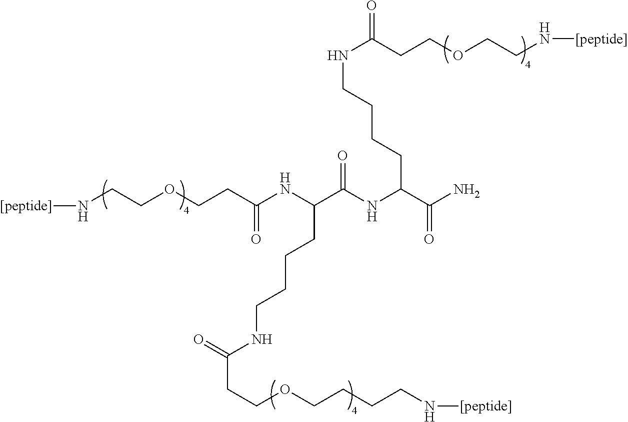

22. The multivalent CoV-2 spike binding peptide of claim 16 having a structure according to Formula (V): 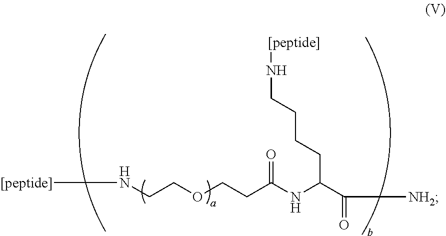
wherein, a, b, and [peptide] are as defined in claim 25.

23. The multivalent Cov-2 spike binding protein of claim 22 having a structure selected from the group consisting of:

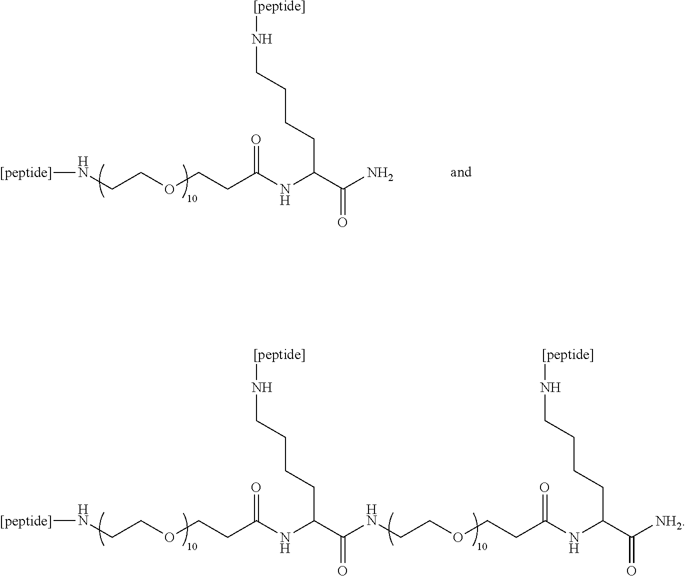

24. The multivalent Cov-2 spike binding protein of claim 16, wherein [peptide] is selected from the group consisting of
(SEQ ID NO: 3)
IEEQAKTFLDKFNHEAEDLFYQS,
(SEQ ID NO: 4)
IEEQAKTFLDKFNHEAEDLFYQ,
(SEQ ID NO: 5)
IEEQAKTFLDKFNHEAEDLFY,
(SEQ ID NO: 6)
IEEQAKTFLDKFNHEAEDLF,
(SEQ ID NO: 7)
IEEQAKTFLDKFNHEAEDL,
(SEQ ID NO: 8)
IEEQAKTFLDKFNHEAED,
(SEQ ID NO: 9)
EEQAKTFLDKFNHEAEDLFYQS,
(SEQ ID NO: 10)
EQAKTFLDKFNHEAEDLFYQS,
(SEQ ID NO: 11)
QAKTFLDKFNHEAEDLFYQS,
(SEQ ID NO: 12)
AKTFLDKFNHEAEDLFYQS,
(SEQ ID NO: 13)
KTFLDKFNHEAEDLFYQS,
(SEQ ID NO: 14)
TFLDKFNHEAEDLFYQS,
(SEQ ID NO: 17)
IEEQAK*TFLDKFNHEAEDLFYQS,
(SEQ ID NO: 18)
IEEQAATFLDKFNHEAEDLFYQS,
(SEQ ID NO: 19)
IEEQASTFLDKFNHEAEDLFYQS,
(SEQ ID NO: 20)
IEEQAQTFLDKFNHEAEDLFYQS,
(SEQ ID NO: 21)
IEEQAKTFLDKFNHEAEDLFYQS,
(SEQ ID NO: 22)
IEEQATTFLDKFNHEAEDLFYQS,
(SEQ ID NO: 23)
IEEQAKTSLDKFNHEAEDLFYQS,
(SEQ ID NO: 24)
IEEQAKTTLDKFNHEAEDLFYQS,
(SEQ ID NO: 25)
IEEQAKTHLDKFNHEAEDLFYQS,
(SEQ ID NO: 26)
IEEQAKTNLDKFNHEAEDLFYQS,
(SEQ ID NO: 27)
IEEQAKTDLDKFNHEAEDLFYQS,
(SEQ ID NO: 28)
IEEQAKTFLDΩFNHEAEDLFYQS,
(SEQ ID NO: 29)
IEEQAzT)KTFLDKFNHEAEDLFYQS,
(SEQ ID NO: 30)
IEEQAKAibFLDKFNHEAEDLFYQS,
(SEQ ID NO: 31)
IEEQAKTFLDKAIbNHEAEDLFYQS,
(SEQ ID NO: 32)
IEEQAKTFLDKFNHEAibEDLFYQS,
(SEQ ID NO: 33)
IEEQAKTFLDKFNHEAEDLAibYQS,
(SEQ ID NO: 16)
STIEEQAKTFLDKFNHEAEDLFYQSS
LASWNYNTNITEENVQNMNNAGDKW
SAFLKEQSTLAQMYPLQEIQ,
(SEQ ID NO: 42)
STIEEQAKTFLDKFNHEAEDLFYQSSL
ASWNYNTNITEENVQNMNNAGDKW
SAFLKEQSTLAQMYPLQEIQNLTVKLQLQALQQN;
(SEQ ID NO: 131)
IEEQAKTFLDKFNHEAEDLFYQSS;
(SEQ ID NO: 45)
IEEQAKTFLDKFNHEAEDLFYQSSLAS;
(SEQ ID NO: 46)
QSTIEEQAKTFLDKF;
(SEQ ID NO: 47)
IEEQAKTFLDKFNHE;
(SEQ ID NO: 48)
QAKTFLDKFNHEAED;
(SEQ ID NO: 49)
TFLDKFNHEAEDLFY;
(SEQ ID NO: 50)
DKFNHEAEDLFYQSS;
(SEQ ID NO: 51)
NHEAEDLFYQSSLAS;
(SEQ ID NO: 52)
QSTIEEQAKTFLDKFNHEAEDLFYQS
SLASWNYNTNITEENVQNMNNAGDK
WSAFLKEQSTLAQMYPLQEIQNLTVK
LQLQALQQNGS;
(SEQ ID NO: 53)
IEEQAKLFLDKFNHEAEDLFYQS;
(SEQ ID NO: 54)
IEEQAKDFLDKFNHEAEDLFYQS;
(SEQ ID NO: 55)
IEEQAKYFLDKFNHEAEDLFYQS;
(SEQ ID NO: 56)
IEEQAKTFLDWFNHEAEDLFYQS;
(SEQ ID NO: 57)
IEEQAKTFLEKFNHEAEDLFYQS;
(SEQ ID NO: 58)
IEEQKKTFEDKFNHEAEDLFYQS;
(SEQ ID NO: 59)
IEEQAKTELDKKNHEAEDLFYQS;
(SEQ ID NO: 60)
IEEQAKTFEDKFKHEAEDLFYQS;
(SEQ ID NO: 61)
IEEQAKTFLDKFKHEEEDKKYQS;
(SEQ ID NO: 62)
IEEQKKTEEDKKKHEEEDKKYQS;
(SEQ ID NO: 63)
IEEQAKTAibLDKFNHEAEDLFYQS;
(SEQ ID NO: 64)
IEEQAKTFLDKFNHEAEDAibFYQS;
(SEQ ID NO: 65)
IEEQAKTFLDKFNHEAEDLFYQAib;
(SEQ ID NO: 66)
FLDWFNHEAEDLFY;
(SEQ ID NO: 67)
LDWFNHEAEDLFY;
(SEQ ID NO: 68)
DWFNHEAEDLFY;
(SEQ ID NO: 69)
TFLDWFNHEAEDLF;
(SEQ ID NO: 70)
TFLDWFNHEAEDL;
(SEQ ID NO: 71)
TFLDWFNHEAEDLFY;
(SEQ ID NO: 72)
TFLCyaWFHEEAEDLFY;
(SEQ ID NO: 73)
TFLDWFNHEAibEDLFY;
(SEQ ID NO: 74)
TFLCyadWFNHEAIbEDLFY;
(SEQ ID NO: 75)
TFLDWFNVEAEDLFY;
(SEQ ID NO: 76)
TFLDWFNFfEAEDLFY;
(SEQ ID NO: 77)
TFLDWFDHEAEDLFY;
(SEQ ID NO: 78)
AFLDKENHEAEDLFY;
(SEQ ID NO: 79)
TALDKFNHEAEDLFY;
(SEQ ID NO: 80)
TFADKENHEAEDLFY;
(SEQ ID NO: 81)
TFLAKFNHEAEDLFY;
(SEQ ID NO: 82)
TFLDAFNHEAEDLFY;
(SEQ ID NO: 83)
TFLDKANHEAEDLFY;
(SEQ ID NO: 84)
TFLDKFAHEAEDLFY;
(SEQ ID NO: 85)
TFLDKFNAEAEDLFY;
(SEQ ID NO: 86)
TFLDKFNHAAEDLFY;
(SEQ ID NO: 87)
TFLDKFNHEAADLFY;
(SEQ ID NO: 88)
TFLDKFNHEAEALFY;
(SEQ ID NO: 89)
TFLDKFNHEAEDAFY;
(SEQ ID NO: 90)
TFLDKFNHEAEDLAY;
(SEQ ID NO: 91)
TFLDKFNHEAEDLFA;
(SEQ ID NO: 92)
EEQAKTFLDKFNHEAEDLFYQSSGGLGKGDFR;
(SEQ ID NO: 93)
EEQAKTFLDKFNHEAEDLFYQSSpLGKGDFR;
(SEQ ID NO: 94)
EEQAKTFLDKFNHEAEDLFYQSSkLGKGDFR;
(SEQ ID NO: 95)
EEQAKTFLCyaKFNHEAEDLFYQSSGLGKGDFR;
(SEQ ID NO: 96)
EEQAKTFLDKFNHEAEDLFYQSSGLGKGCyaGFR;
(SEQ ID NO: 97)
EEQAKTFLDKFNFfEAEDLFYQSSGLGKGDFR;
(SEQ ID NO: 98)
EEQAKTFLDKFNVEAEDLFYQSSGLGKGDFR;
(SEQ ID NO: 101)
AQMYPLQEGIEEQAKTFLDKFNHEAEDLFYQSSGLGKGDFR;
(SEQ ID NO: 102)
AQMYPLQEGGIEEQAKTFLDKFNHEAEDLFYQSSGLGKGDFR;
(SEQ ID NO: 103)
AQMYPLQEGIEEQAKTFLDKFNHEAEDLFYQSS;
(SEQ ID NO: 104)
YFLDWFNHEAEDLFY;
(SEQ ID NO: 105)
TFLDWFNHEAEDKFY;
(SEQ ID NO: 106)
TFLDWFNAEAEDLFY;
(SEQ ID NO: 107)
TFLDWFNAibEAEDLFY;
(SEQ ID NO: 108)
TFLDWFNHEAEDOrnFY;
(SEQ ID NO: 109)
TFLDWFNHEAEDPipFY;
(SEQ ID NO: 110)
TFLDWFNHEPipEDLFY;
(SEQ ID NO: 111)
TFLDWFNPipEAEDLFY;
(SEQ ID NO: 112)
TFLDWFNHEAEDLFYK;
(SEQ ID NO: 113)
SLDQINVTFLDLEYEMKKLEEAIKKL
EESYIDLKELGSGSGSGSGSTFLDWF
NHEAEDLFY;
(SEQ ID NO: 114)
SLDQINVTFLDLEYEMKKLEEAIKKLE
ESYIDLKELGSGRRARSGSTFLDWFN
HEAEDLFY;
(SEQ ID NO: 115)
TFLDΩ₁FNHEAEDLFY;
(SEQ ID NO: 116)
TFLDΩ₂FNHEAEDLFY;
(SEQ ID NO: 117)
TFLDΩ₃FNHEAEDLFY;
(SEQ ID NO: 118)
TFLDΩ₄FNHEAEDLFY;
(SEQ ID NO: 119)
TFLDΩ₅FNHEAEDLFY;
(SEQ ID NO: 120)
TFLDΩ₆FNHEAEDLFY;
(SEQ ID NO: 121)
TFLDΩ₇FNHEAEDLFY;
(SEQ ID NO: 122)
TFLDΩ₈FNHEAEDLFY;
(SEQ ID NO: 123)
TFLDΩ₉FNHEAEDLFY;
(SEQ ID NO: 124)
TFLDΩ₁₀FNHEAEDLFY;
(SEQ ID NO: 125)
TFLDΩ₁₁FNHEAEDLFY;
(SEQ ID NO: 126)
TFLDΩ₁₂FNHEAEDLFY;
(SEQ ID NO: 127)
TFLDΩ₁₃FNHEAEDLFY;
(SEQ ID NO: 128)
TFLDΩ₁₄FNHEAEDLFY;
(SEQ ID NO: 129)
TFLDΩ₁₅FNHEAEDLFY;
and
(SEQ ID NO: 130)
TFLDΩ₁₆FNHEAEDLFY.

25. The multivalent Cov-2 spike binding protein of claim 16, wherein [peptide] is selected from the group consisting of
(SEQ ID NO: 131)
IEEQAKTFLDKFNHEAEDLFYQSS
and
(SEQ ID NO: 132)
TFLDWFNHEAEDLFY.

26. A pharmaceutical composition comprising the multivalent Cov-2 spike binding protein of claim 16 and a pharmaceutically acceptable carrier.

27. The pharmaceutical composition of claim 26, wherein the multivalent Cov-2 spike binding protein specifically binds to a receptor binding domain (RBD) of the spike protein.

28. The pharmaceutical composition of claim 26, wherein the multivalent Cov-2 spike binding protein specifically binds to human ACE2 receptor.

29. The pharmaceutical composition of claim 26, wherein the RBD specifically binds to human ACE2 receptor.

30. The pharmaceutical composition of claim 26, wherein the coronavirus is selected from the group consisting of human coronavirus 229E (HCoV-229E), human coronavirus OC43 (HCoV-OC43), severe acute respiratory syndrome coronavirus (SARS-CoV), human coronavirus NL63 (HCoV-NL63), human coronavirus HKU1, Middle East respiratory syndrome-related coronavirus (MERS-CoV) and severe acute respiratory syndrome coronavirus 2 (SARS-CoV-2).

31. The pharmaceutical composition of claim 30, wherein the coronavirus is SARS-CoV-2.

32. The pharmaceutical composition of claim 26, wherein the peptide binds the spike protein receptor binding domain of the coronavirus with a K_{D} of less than about 500 nM.

33. A pharmaceutical composition comprising one or more peptide of claim 1 and a pharmaceutically acceptable carrier.

34. A pharmaceutical composition comprising one or more peptide of claim 10 and a pharmaceutically acceptable carrier.

35. A method of reducing probability of viral infection in a human subject, comprising administering a therapeutically effective amount of the pharmaceutical composition of claim 34.

36. The method of claim 35, wherein the viral infection is caused by a coronavirus.

37. The method of claim 35, wherein the coronavirus is selected from the group consisting of HCoV-229E, HCoV-OC43, SARS-CoV, HCoV-NL63, human coronavirus HKU1, MERS-CoV and SARS-CoV-2.

38. The method of claim 35, wherein the coronavirus is SARS-CoV-2.

39. The method of claim 35, wherein the administration of the pharmaceutical composition comprises intradermal, intramuscular, intraperitoneal, intravenous, subcutaneous, intranasal, epidural, sublingual, intracerebral, intravaginal, transdermal, rectal, by inhalation, or topical administration.

40. The method of claim 35, wherein the human subject is at least 50 years of age.

41. A method of detecting the presence of a viral infection in a human subject in need thereof, comprising
(a) contacting a sample from the subject with the composition of claim 34, and
(b) detecting specific binding between the composition and an antibody that specifically binds the infecting virus,
wherein specific binding between the antibody and the composition indicates the presence of viral infection in the subject.

42. The method of claim 41, wherein the peptide is covalently bound to a solid phase substrate.

43. The method of claim 42, wherein the detection of the detecting specific binding between the composition and the virus is performed using an immunoassay.

44. The method of claim 42, wherein the immunoassay is an enzyme-linked immunosorbent assay (ELISA).

45. The method of claim 41, wherein the viral infection is caused by a coronavirus.

46. The method of claim 41, wherein the coronavirus is selected from the group consisting of HCoV-229E, HCoV-OC43, SARS-CoV, HCoV-NL63, human coronavirus HKU1, MERS-CoV and SARS-CoV-2.

47. The method of claim 41, wherein the coronavirus is SARS-CoV-2.

48. The method of claim 41, wherein the subject may not have a symptom, or may have a symptom associated with viral infection selected from the group consisting of fever, cough, shortness of breath, pain or pressure in the chest, confusion, bluish lips or face, pneumonia, bronchitis, runny nose, sneezing, chills, exacerbated asthma, acute respiratory distress syndrome (ARDS), RNAaemia, acute cardiac injury, shock, myalgia, fatigue, sputum production, rusty colored sputum, bloody sputum, swelling of lymph nodes, middle ear infection, joint pain, wheezing, headache, hemoptysis, diarrhea, dyspnea, redness, swelling or edema, pain, loss of function, organ dysfunction, multi-organ system failure, acute kidney injury, malnutrition, sepsis, hypotension, hypertension, hypothermia, hypoxemia, leukocytosis, leukopenia, lymphopenia, thrombocytopenia, nasal congestion, sore throat, unwillingness to drink, convulsions, ongoing vomiting, abdominal pain, secondary infection, cytokine release syndrome, and multi-organ failure.

49. The method of claim 41, wherein the human subject is at least 50 years of age.

50. A kit comprising the composition of claim 34 and a solid phase substrate.

51. The kit of claim 50 further comprising a monoclonal antibody that specifically binds to a human Ig constant domain.

52. The kit of claim 50, wherein the monoclonal antibody comprises a detectable marker.


SEQUENCE LISTING (143 sequences)

SEQ ID NO: 1 - protein, 223 aa, Severe acute respiratory syndrome coronavirus 2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGPNCYFPLQSYGFQPTNGVGYQPYRVVVLSFELLHAPATVCGPKKSTNLVKNKCVNF

SEQ ID NO: 2 - protein, 805 aa, Homo sapiens [sequence omitted]

SEQ ID NO: 3 - protein, 23 aa, Artificial Sequence
  Description of Artificial Sequence Synthetic peptide
IEEQAKTFLDKFNHEAEDLFYQS

SEQ ID NO: 4 - protein, 22 aa, Artificial Sequence
  Description of Artificial Sequence Synthetic peptide
IEEQAKTFLDKFNHEAEDLFYQ

SEQ ID NO: 5 - protein, 21 aa, Artificial Sequence
  Description of Artificial Sequence Synthetic peptide
IEEQAKTFLDKFNHEAEDLFY

SEQ ID NO: 6 - protein, 20 aa, Artificial Sequence
  Description of Artificial Sequence Synthetic peptide
IEEQAKTFLDKFNHEAEDLF

SEQ ID NO: 7 - protein, 19 aa, Artificial Sequence
  Description of Artificial Sequence Synthetic peptide
IEEQAKTFLDKFNHEAEDL

SEQ ID NO: 8 - protein, 18 aa, Artificial Sequence
  Description of Artificial Sequence Synthetic peptide
IEEQAKTFLDKFNHEAED

SEQ ID NO: 9 - protein, 22 aa, Artificial Sequence
  Description of Artificial Sequence Synthetic peptide
EEQAKTFLDKFNHEAEDLFYQS

SEQ ID NO: 10 - protein, 21 aa, Artificial Sequence
  Description of Artificial Sequence Synthetic peptide
EQAKTFLDKFNHEAEDLFYQS

SEQ ID NO: 11 - protein, 20 aa, Artificial Sequence
  Description of Artificial Sequence Synthetic peptide
QAKTFLDKFNHEAEDLFYQS

SEQ ID NO: 12 - protein, 19 aa, Artificial Sequence
  Description of Artificial Sequence Synthetic peptide
AKTFLDKFNHEAEDLFYQS

SEQ ID NO: 13 - protein, 18 aa, Artificial Sequence
  Description of Artificial Sequence Synthetic peptide
KTFLDKFNHEAEDLFYQS

SEQ ID NO: 14 - protein, 17 aa, Artificial Sequence
  Description of Artificial Sequence Synthetic peptide
TFLDKFNHEAEDLFYQS

SEQ ID NO: 15 - protein, 12 aa, Artificial Sequence
  Description of Artificial Sequence Synthetic peptide
TFLDKFNHEAED

SEQ ID NO: 16 - protein, 71 aa, Artificial Sequence
  Description of Artificial Sequence Synthetic polypeptide
STIEEQAKTFLDKFNHEAEDLFYQSSLASWNYNTNITEENVQNMNNAGDKWSAFLKEQSTLAQMYPLQEIQ

SEQ ID NO: 17 - protein, 23 aa, Artificial Sequence
  Description of Artificial Sequence Synthetic peptide
  MOD_RES (6)..(6): Modified Lys (K*)
IEEQAKTFLDKFNHEAEDLFYQS

SEQ ID NO: 18 - protein, 23 aa, Artificial Sequence
  Description of Artificial Sequence Synthetic peptide
IEEQAATFLDKFNHEAEDLFYQS

SEQ ID NO: 19 - protein, 23 aa, Artificial Sequence
  Description of Artificial Sequence Synthetic peptide
IEEQASTFLDKFNHEAEDLFYQS

SEQ ID NO: 20 - protein, 23 aa, Artificial Sequence
  Description of Artificial Sequence Synthetic peptide
IEEQAQTFLDKFNHEAEDLFYQS

SEQ ID NO: 21 - protein, 23 aa, Artificial Sequence
  Description of Artificial Sequence Synthetic peptide
IEEQAHTFLDKFNHEAEDLFYQS

SEQ ID NO: 22 - protein, 23 aa, Artificial Sequence
  Description of Artificial Sequence Synthetic peptide
IEEQATTFLDKFNHEAEDLFYQS

SEQ ID NO: 23 - protein, 23 aa, Artificial Sequence
  Description of Artificial Sequence Synthetic peptide
IEEQAKTSLDKFNHEAEDLFYQS

SEQ ID NO: 24 - protein, 23 aa, Artificial Sequence
  Description of Artificial Sequence Synthetic peptide
IEEQAKTTLDKFNHEAEDLFYQS

SEQ ID NO: 25 - protein, 23 aa, Artificial Sequence
  Description of Artificial Sequence Synthetic peptide
IEEQAKTHLDKFNHEAEDLFYQS

SEQ ID NO: 26 - protein, 23 aa, Artificial Sequence
  Description of Artificial Sequence Synthetic peptide
IEEQAKTNLDKFNHEAEDLFYQS

SEQ ID NO: 27 - protein, 23 aa, Artificial Sequence
  Description of Artificial Sequence Synthetic peptide
IEEQAKTDLDKFNHEAEDLFYQS

SEQ ID NO: 28 - protein, 23 aa, Artificial Sequence
  Description of Artificial Sequence Synthetic peptide
IEEQAKTFLDQFNHEAEDLFYQS

SEQ ID NO: 29 - protein, 23 aa, Artificial Sequence
  Description of Artificial Sequence Synthetic peptide
  MOD_RES (5)..(5): Aib
IEEQXKTFLDKFNHEAEDLFYQS

SEQ ID NO: 30 - protein, 23 aa, Artificial Sequence
  Description of Artificial Sequence Synthetic peptide
  MOD_RES (7)..(7): Aib
IEEQAKXFLDKFNHEAEDLFYQS

SEQ ID NO: 31 - protein, 23 aa, Artificial Sequence
  Description of Artificial Sequence Synthetic peptide
  MOD_RES (12)..(12): Aib
IEEQAKTFLDKXNHEAEDLFYQS

SEQ ID NO: 32 - protein, 23 aa, Artificial Sequence
  Description of Artificial Sequence Synthetic peptide
  MOD_RES (16)..(16): Aib
IEEQAKTFLDKFNHEXEDLFYQS

SEQ ID NO: 33 - protein, 23 aa, Artificial Sequence
  Description of Artificial Sequence Synthetic peptide
  MOD_RES (20)..(20): Aib
IEEQAKTFLDKFNHEAEDLXYQS

SEQ ID NO: 34 - protein, 23 aa, Artificial Sequence
  Description of Artificial Sequence Synthetic peptide
  MOD_RES (5)..(5): Any amino acid
  MOD_RES (5)..(8): Residues at positions 5 and 8 may be stapled
  MOD_RES (8)..(8): Any amino acid
IEEQXKTXLDKFNHEAEDLFYQS

SEQ ID NO: 35 - protein, 23 aa, Artificial Sequence
  Description of Artificial Sequence Synthetic peptide
  MOD_RES (5)..(5): Any amino acid
  MOD_RES (5)..(12): Residues at positions 5 and 12 may be stapled
  MOD_RES (12)..(12): Any amino acid
IEEQXKTFLDKXNHEAEDLFYQS

SEQ ID NO: 36 - protein, 23 aa, Artificial Sequence
  Description of Artificial Sequence Synthetic peptide
  MOD_RES (8)..(8): Any amino acid
  MOD_RES (8)..(12): Residues at positions 8 and 12 may be stapled
  MOD_RES (12)..(12): Any amino acid
IEEQAKTXLDKXNHEAEDLFYQS

SEQ ID NO: 37 - protein, 23 aa, Artificial Sequence
  Description of Artificial Sequence Synthetic peptide
  MOD_RES (8)..(8): Any amino acid
  MOD_RES (8)..(16): Residues at positions 8 and 16 may be stapled
  MOD_RES (16)..(16): Any amino acid
IEEQAKTXLDKFNHEXEDLFYQS

SEQ ID NO: 38 - protein, 23 aa, Artificial Sequence
  Description of Artificial Sequence Synthetic peptide
  MOD_RES (12)..(12): Any amino acid
  MOD_RES (12)..(16): Residues at positions 12 and 16 may be stapled
  MOD_RES (16)..(16): Any amino acid
IEEQAKTFLDKXNHEXEDLFYQS

SEQ ID NO: 39 - protein, 23 aa, Artificial Sequence
  Description of Artificial Sequence Synthetic peptide
  MOD_RES (16)..(16): Any amino acid
  MOD_RES (16)..(20): Residues at positions 16 and 20 may be stapled
  MOD_RES (20)..(20): Any amino acid
IEEQAKTFLDKFNHEXEDLXYQS

SEQ ID NO: 40 - protein, 17 aa, Artificial Sequence
  Description of Artificial Sequence Synthetic peptide
IEEQAKTFLDKFNHEAE

SEQ ID NO: 41 - protein, 8 aa, Artificial Sequence
  Description of Artificial Sequence Synthetic peptide
TCLDKCNH

SEQ ID NO: 42 - protein, 85 aa, Artificial Sequence
  Description of Artificial Sequence Synthetic polypeptide
STIEEQAKTFLDKFNHEAEDLFYQSSLASWNYNTNITEENVQNMNNAGDKWSAFLKEQSTLAQMYPLQEIQNLTVKLQLQALQQN

SEQ ID NO: 43 - protein, 23 aa, Artificial Sequence
  Description of Artificial Sequence Synthetic peptide
EQAEKDNFHILKEYAQTDEFFSL

SEQ ID NO: 44 - protein, 23 aa, Artificial Sequence
  Description of Artificial Sequence Synthetic peptide
AIETAQSEHEKFQLNDYLDFKFE

SEQ ID NO: 45 - protein, 27 aa, Artificial Sequence
  Description of Artificial Sequence Synthetic peptide
IEEQAKTFLDKFNHEAEDLFYQSSLAS

SEQ ID NO: 46 - protein, 15 aa, Artificial Sequence
  Description of Artificial Sequence Synthetic peptide
QSTIEEQAKTFLDKF

SEQ ID NO: 47 - protein, 15 aa, Artificial Sequence
  Description of Artificial Sequence Synthetic peptide
IEEQAKTFLDKFNHE

SEQ ID NO: 48 - protein, 15 aa, Artificial Sequence
  Description of Artificial Sequence Synthetic peptide
QAKTFLDKFNHEAED

SEQ ID NO: 49 - protein, 15 aa, Artificial Sequence
  Description of Artificial Sequence Synthetic peptide
TFLDKFNHEAEDLFY

SEQ ID NO: 50 - protein, 15 aa, Artificial Sequence
  Description of Artificial Sequence Synthetic peptide
DKFNHEAEDLFYQSS

SEQ ID NO: 51 - protein, 15 aa, Artificial Sequence
  Description of Artificial Sequence Synthetic peptide
NHEAEDLFYQSSLAS

SEQ ID NO: 52 - protein, 88 aa, Artificial Sequence
  Description of Artificial Sequence Synthetic polypeptide
QSTIEEQAKTFLDKFNHEAEDLFYQSSLASWNYNTNITEENVQNMNNAGDKWSAFLKEQSTLAQMYPLQEIQNLTVKLQLQALQQNGS

SEQ ID NO: 53 - protein, 23 aa, Artificial Sequence
  Description of Artificial Sequence Synthetic peptide
IEEQAKLFLDKFNHEAEDLFYQS

SEQ ID NO: 54 - protein, 23 aa, Artificial Sequence
  Description of Artificial Sequence Synthetic peptide
IEEQAKDFLDKFNHEAEDLFYQS

SEQ ID NO: 55 - protein, 23 aa, Artificial Sequence
  Description of Artificial Sequence Synthetic peptide
IEEQAKYFLDKFNHEAEDLFYQS

SEQ ID NO: 56 - protein, 23 aa, Artificial Sequence
  Description of Artificial Sequence Synthetic peptide
IEEQAKTFLDWFNHEAEDLFYQS

SEQ ID NO: 57 - protein, 23 aa, Artificial Sequence
  Description of Artificial Sequence Synthetic peptide
IEEQAKTFLEKFNHEAEDLFYQS

SEQ ID NO: 58 - protein, 23 aa, Artificial Sequence
  Description of Artificial Sequence Synthetic peptide
IEEQKKTFEDKFNHEAEDLFYQS

SEQ ID NO: 59 - protein, 23 aa, Artificial Sequence
  Description of Artificial Sequence Synthetic peptide
IEEQAKTELDKKNHEAEDLFYQS

SEQ ID NO: 60 - protein, 23 aa, Artificial Sequence
  Description of Artificial Sequence Synthetic peptide
IEEQAKTFEDKFKHEAEDLFYQS

SEQ ID NO: 61 - protein, 23 aa, Artificial Sequence
  Description of Artificial Sequence Synthetic peptide
IEEQAKTFLDKFKHEEEDKKYQS

SEQ ID NO: 62 - protein, 23 aa, Artificial Sequence
  Description of Artificial Sequence Synthetic peptide
IEEQKKTEEDKKKHEEEDKKYQS

SEQ ID NO: 63 - protein, 23 aa, Artificial Sequence
  Description of Artificial Sequence Synthetic peptide
  MOD_RES (8)..(8): Aib
IEEQAKTXLDKFNHEAEDLFYQS

SEQ ID NO: 64 - protein, 23 aa, Artificial Sequence
  Description of Artificial Sequence Synthetic peptide
  MOD_RES (19)..(19): Aib
IEEQAKTFLDKFNHEAEDXFYQS

SEQ ID NO: 65 - protein, 23 aa, Artificial Sequence
  Description of Artificial Sequence Synthetic peptide
  MOD_RES (23)..(23): Aib
IEEQAKTFLDKFNHEAEDLFYQX

SEQ ID NO: 66 - protein, 14 aa, Artificial Sequence
  Description of Artificial Sequence Synthetic peptide
FLDWFNHEAEDLFY

SEQ ID NO: 67 - protein, 13 aa, Artificial Sequence
  Description of Artificial Sequence Synthetic peptide
LDWFNHEAEDLFY

SEQ ID NO: 68 - protein, 12 aa, Artificial Sequence
  Description of Artificial Sequence Synthetic peptide
DWFNHEAEDLFY

SEQ ID NO: 69 - protein, 14 aa, Artificial Sequence
  Description of Artificial Sequence Synthetic peptide
TFLDWFNHEAEDLF

SEQ ID NO: 70 - protein, 13 aa, Artificial Sequence
  Description of Artificial Sequence Synthetic peptide
TFLDWFNHEAEDL

SEQ ID NO: 71 - protein, 15 aa, Artificial Sequence
  Description of Artificial Sequence Synthetic peptide
TFLDWFNHEAEDLFY

SEQ ID NO: 72 - protein, 15 aa, Artificial Sequence
  Description of Artificial Sequence Synthetic peptide
  MOD_RES (4)..(4): Cya
TFLXWFHEEAEDLFY

SEQ ID NO: 73 - protein, 15 aa, Artificial Sequence
  Description of Artificial Sequence Synthetic peptide
  MOD_RES (10)..(10): Aib
TFLDWFNHEXEDLFY

SEQ ID NO: 74 - protein, 15 aa, Artificial Sequence
  Description of Artificial Sequence Synthetic peptide
  MOD_RES (4)..(4): Cya
  MOD_RES (10)..(10): Aib
TFLXWFNHEXEDLFY

SEQ ID NO: 75 - protein, 15 aa, Artificial Sequence
  Description of Artificial Sequence Synthetic peptide
TFLDWFNVEAEDLFY

SEQ ID NO: 76 - protein, 15 aa, Artificial Sequence
  Description of Artificial Sequence Synthetic peptide
  MOD_RES (8)..(8): 4-fluorophenylalanine
TFLDWFNFEAEDLFY

SEQ ID NO: 77 - protein, 15 aa, Artificial Sequence
  Description of Artificial Sequence Synthetic peptide
TFLDWFDHEAEDLFY

SEQ ID NO: 78 - protein, 15 aa, Artificial Sequence
  Description of Artificial Sequence Synthetic peptide
AFLDKFNHEAEDLFY

SEQ ID NO: 79 - protein, 15 aa, Artificial Sequence
  Description of Artificial Sequence Synthetic peptide
TALDKFNHEAEDLFY

SEQ ID NO: 80 - protein, 15 aa, Artificial Sequence
  Description of Artificial Sequence Synthetic peptide
TFADKFNHEAEDLFY

SEQ ID NO: 81 - protein, 15 aa, Artificial Sequence
  Description of Artificial Sequence Synthetic peptide
TFLAKFNHEAEDLFY

SEQ ID NO: 82 - protein, 15 aa, Artificial Sequence
  Description of Artificial Sequence Synthetic peptide
TFLDAFNHEAEDLFY

SEQ ID NO: 83 - protein, 15 aa, Artificial Sequence
  Description of Artificial Sequence Synthetic peptide
TFLDKANHEAEDLFY

SEQ ID NO: 84 - protein, 15 aa, Artificial Sequence
  Description of Artificial Sequence Synthetic peptide
TFLDKFAHEAEDLFY

SEQ ID NO: 85 - protein, 15 aa, Artificial Sequence
  Description of Artificial Sequence Synthetic peptide
TFLDKFNAEAEDLFY

SEQ ID NO: 86 - protein, 15 aa, Artificial Sequence
  Description of Artificial Sequence Synthetic peptide
TFLDKFNHAAEDLFY

SEQ ID NO: 87 - protein, 15 aa, Artificial Sequence
  Description of Artificial Sequence Synthetic peptide
TFLDKFNHEAADLFY

SEQ ID NO: 88 - protein, 15 aa, Artificial Sequence
  Description of Artificial Sequence Synthetic peptide
TFLDKFNHEAEALFY

SEQ ID NO: 89 - protein, 15 aa, Artificial Sequence
  Description of Artificial Sequence Synthetic peptide
TFLDKFNHEAEDAFY

SEQ ID NO: 90 - protein, 15 aa, Artificial Sequence
  Description of Artificial Sequence Synthetic peptide
TFLDKFNHEAEDLAY

SEQ ID NO: 91 - protein, 15 aa, Artificial Sequence
  Description of Artificial Sequence Synthetic peptide
TFLDKFNHEAEDLFA

SEQ ID NO: 92 - protein, 32 aa, Artificial Sequence
  Description of Artificial Sequence Synthetic polypeptide
EEQAKTFLDKFNHEAEDLFYQSSGGLGKGDFR

SEQ ID NO: 93 - protein, 31 aa, Artificial Sequence
  Description of Artificial Sequence Synthetic polypeptide
  MOD_RES (24)..(24): D-amino acid
EEQAKTFLDKFNHEAEDLFYQSSPLGKGDFR

SEQ ID NO: 94 - protein, 31 aa, Artificial Sequence
  Description of Artificial Sequence Synthetic polypeptide
  MOD_RES (24)..(24): D-amino acid
EEQAKTFLDKFNHEAEDLFYQSSKLGKGDFR

SEQ ID NO: 95 - protein, 31 aa, Artificial Sequence
  Description of Artificial Sequence Synthetic polypeptide
  MOD_RES (9)..(9): Cya
EEQAKTFLXKFNHEAEDLFYQSSGLGKGDFR

SEQ ID NO: 96 - protein, 31 aa, Artificial Sequence
  Description of Artificial Sequence Synthetic polypeptide
  MOD_RES (29)..(29): Cya
EEQAKTFLDKFNHEAEDLFYQSSGLGKGXFR

SEQ ID NO: 97 - protein, 31 aa, Artificial Sequence
  Description of Artificial Sequence Synthetic polypeptide
  MOD_RES (13)..(13): 4-fluorophenylalanine
EEQAKTFLDKFNFEAEDLFYQSSGLGKGDFR

SEQ ID NO: 98 - protein, 31 aa, Artificial Sequence
  Description of Artificial Sequence Synthetic polypeptide
EEQAKTFLDKFNVEAEDLFYQSSGLGKGDFR

SEQ ID NO: 99 - protein, 8 aa, Artificial Sequence
  Description of Artificial Sequence Synthetic peptide
GLGKGDFR

SEQ ID NO: 100 - protein, 9 aa, Artificial Sequence
  Description of Artificial Sequence Synthetic peptide
AQMYPLQEG

SEQ ID NO: 101 - protein, 41 aa, Artificial Sequence
  Description of Artificial Sequence Synthetic polypeptide
AQMYPLQEGIEEQAKTFLDKFNHEAEDLFYQSSGLGKGDFR

SEQ ID NO: 102 - protein, 42 aa, Artificial Sequence
  Description of Artificial Sequence Synthetic polypeptide
AQMYPLQEGGIEEQAKTFLDKFNHEAEDLFYQSSGLGKGDFR

SEQ ID NO: 103 - protein, 33 aa, Artificial Sequence
  Description of Artificial Sequence Synthetic polypeptide
AQMYPLQEGIEEQAKTFLDKFNHEAEDLFYQSS

SEQ ID NO: 104 - protein, 15 aa, Artificial Sequence
  Description of Artificial Sequence Synthetic peptide
YFLDWFNHEAEDLFY

SEQ ID NO: 105 - protein, 15 aa, Artificial Sequence
  Description of Artificial Sequence Synthetic peptide
TFLDWFNHEAEDKFY

SEQ ID NO: 106 - protein, 15 aa, Artificial Sequence
  Description of Artificial Sequence Synthetic peptide
TFLDWFNAEAEDLFY

SEQ ID NO: 107 - protein, 15 aa, Artificial Sequence
  Description of Artificial Sequence Synthetic peptide
  MOD_RES (8)..(8): Aib
TFLDWFNXEAEDLFY

SEQ ID NO: 108 - protein, 15 aa, Artificial Sequence
  Description of Artificial Sequence Synthetic peptide
  MOD_RES (13)..(13): Orn
TFLDWFNHEAEDXFY

SEQ ID NO: 109 - protein, 15 aa, Artificial Sequence
  Description of Artificial Sequence Synthetic peptide
  MOD_RES (13)..(13): Pip
TFLDWFNHEAEDXFY

SEQ ID NO: 110 - protein, 15 aa, Artificial Sequence
  Description of Artificial Sequence Synthetic peptide
  MOD_RES (10)..(10): Pip
TFLDWFNHEXEDLFY

SEQ ID NO: 111 - protein, 15 aa, Artificial Sequence
  Description of Artificial Sequence Synthetic peptide
  MOD_RES (8)..(8): Pip
TFLDWFNXEAEDLFY

SEQ ID NO: 112 - protein, 16 aa, Artificial Sequence
  Description of Artificial Sequence Synthetic peptide
TFLDWFNHEAEDLFYK

SEQ ID NO: 113 - protein, 61 aa, Artificial Sequence
  Description of Artificial Sequence Synthetic polypeptide
SLDQINVTFLDLEYEMKKLEEAIKKLEESYIDLKELGSGSGSGSGSTFLDWFNHEAEDLFY

SEQ ID NO: 114 - protein, 61 aa, Artificial Sequence
  Description of Artificial Sequence Synthetic polypeptide
SLDQINVTFLDLEYEMKKLEEAIKKLEESYIDLKELGSGRRARSGSTFLDWFNHEAEDLFY

SEQ ID NO: 115 - protein, 15 aa, Artificial Sequence
  Description of Artificial Sequence Synthetic peptide
  MOD_RES (5)..(5): 4-phenyl-L-phenylalanine
TFLDFFNHEAEDLFY

SEQ ID NO: 116 - protein, 15 aa, Artificial Sequence
  Description of Artificial Sequence Synthetic peptide
  MOD_RES (5)..(5): 4-fluoro-L-phenylalanine
TFLDFFNHEAEDLFY

SEQ ID NO: 117 - protein, 15 aa, Artificial Sequence
  Description of Artificial Sequence Synthetic peptide
  MOD_RES (5)..(5): 4-bromo-L-phenylalanine
TFLDFFNHEAEDLFY

SEQ ID NO: 118 - protein, 15 aa, Artificial Sequence
  Description of Artificial Sequence Synthetic peptide
  MOD_RES (5)..(5): 3-cyano-L-phenylalanine
TFLDFFNHEAEDLFY

SEQ ID NO: 119 - protein, 15 aa, Artificial Sequence
  Description of Artificial Sequence Synthetic peptide
  MOD_RES (5)..(5): 4-amino-L-phenylalanine
TFLDFFNHEAEDLFY

SEQ ID NO: 120 - protein, 15 aa, Artificial Sequence
  Description of Artificial Sequence Synthetic peptide
  MOD_RES (5)..(5): 3,4,5-trifluoro-L-phenylalanine
TFLDFFNHEAEDLFY

SEQ ID NO: 121 - protein, 15 aa, Artificial Sequence
  Description of Artificial Sequence Synthetic peptide
  MOD_RES (5)..(5): 2,4,5-trifluoro-L-phenylalanine
TFLDFFNHEAEDLFY

SEQ ID NO: 122 - protein, 15 aa, Artificial Sequence
  Description of Artificial Sequence Synthetic peptide
  MOD_RES (5)..(5): Nitrotyrosine
TFLDYFNHEAEDLFY

SEQ ID NO: 123 - protein, 15 aa, Artificial Sequence
  Description of Artificial Sequence Synthetic peptide
  MOD_RES (5)..(5): L-4-pyridylalanine
TFLDAFNHEAEDLFY

SEQ ID NO: 124 - protein, 15 aa, Artificial Sequence
  Description of Artificial Sequence Synthetic peptide
  MOD_RES (5)..(5): L-2-naphthylalanine
TFLDAFNHEAEDLFY

SEQ ID NO: 125 - protein, 15 aa, Artificial Sequence
  Description of Artificial Sequence Synthetic peptide
  MOD_RES (5)..(5): L-1-naphthylalanine
TFLDAFNHEAEDLFY

SEQ ID NO: 126 - protein, 15 aa, Artificial Sequence
  Description of Artificial Sequence Synthetic peptide
  MOD_RES (5)..(5): (S)-2-amino-cyclopropanepropanoic acid
TFLDXFNHEAEDLFY

SEQ ID NO: 127 - protein, 15 aa, Artificial Sequence
  Description of Artificial Sequence Synthetic peptide
  MOD_RES (5)..(5): (S)-2-amino-3-cyclobutylpropanoic acid
TFLDXFNHEAEDLFY

SEQ ID NO: 128 - protein, 15 aa, Artificial Sequence
  Description of Artificial Sequence Synthetic peptide
  MOD_RES (5)..(5): L-cyclohexylalanine
TFLDAFNHEAEDLFY

SEQ ID NO: 129 - protein, 15 aa, Artificial Sequence
  Description of Artificial Sequence Synthetic peptide
  MOD_RES (5)..(5): L-homophenylalanine
TFLDFFNHEAEDLFY

SEQ ID NO: 130 - protein, 15 aa, Artificial Sequence
  Description of Artificial Sequence Synthetic peptide
  MOD_RES (5)..(5): 4-(aminomethyl)-l-phenylalanine
TFLDFFNHEAEDLFY

SEQ ID NO: 131 - protein, 24 aa, Artificial Sequence
  Description of Artificial Sequence Synthetic peptide
IEEQAKTFLDKFNHEAEDLFYQSS

SEQ ID NO: 132 - protein, 15 aa, Artificial Sequence
  Description of Artificial Sequence Synthetic peptide
TFLDWFNHEAEDLFY

SEQ ID NO: 133 - protein, 23 aa, Artificial Sequence
  Description of Artificial Sequence Synthetic peptide
  MOD_RES (1)..(1): I or absent
  SITE (1)..(6): When positions 2-6 are absent, all previous residues are also absent
  SITE (1)..(23): The residue at position 5 may be stapled with the residue at either position 8 or 12
  SITE (1)..(23): The residue at position 8 may be stapled with the residue at either position 5, 12, or 16
  SITE (1)..(23): The residue at position 12 may be stapled with the residue at either position 5, 8, or 16
  SITE (1)..(23): The residue at position 16 may be stapled with the residue at either position 8, 12, or 20
  SITE (1)..(23): The residue at position 20 may be stapled with the residue at position 16
  MOD_RES (2)..(2): E or absent
  MOD_RES (3)..(3): E or absent
  MOD_RES (4)..(4): Q or absent
  MOD_RES (5)..(5): A, Aib, or absent
  MOD_RES (6)..(6): K, modified Lys (K*), A, S, Q, H, T, or absent
  MOD_RES (7)..(7): T or Aib
  MOD_RES (8)..(8): F, S, T, H, N, or D
  MOD_RES (11)..(11): K or Q
  MOD_RES (12)..(12): F or Aib
  MOD_RES (16)..(16): A or Aib
  MOD_RES (19)..(19): L or absent
  SITE (19)..(23): When positions 19-22 are absent, all subsequent residues are also absent
  MOD_RES (20)..(20): F, Aib, or absent
  MOD_RES (21)..(21): Y or absent
  MOD_RES (22)..(22): Q or absent
  MOD_RES (23)..(23): S or absent
  See specification as filed for detailed description of substitutions and preferred embodiments
XXXXXXXXLDXXNHEXEDXXXXX

SEQ ID NO: 134 - protein, 43 aa, Artificial Sequence
  Description of Artificial Sequence Synthetic polypeptide
  MOD_RES (1)..(10): This region may encompass one of the following sequences “QST” or “AQMYPLQEG” or “AQMYPLQEGG” or absent
  SITE (1)..(22): When any of positions 11-22 are absent, all residues prior to the absent residue are also absent
  SITE (1)..(43): Wherein at least 12 consecutive residues are not absent
  SITE (1)..(43): The residue at position 15 may be stapled with the residue at either position 18 or 22
  SITE (1)..(43): The residue at position 18 may be stapled with the residue at either position 15, 22, or 26
  SITE (1)..(43): The residue at position 22 may be stapled with the residue at either position 15, 18, or 26
  SITE (1)..(43): The residue at position 26 may be stapled with the residue at either position 18, 22, or 30
  SITE (1)..(43): The residue at position 30 may be stapled with the residue at position 26
  MOD_RES (11)..(11): I or absent
  MOD_RES (12)..(12): E or absent
  MOD_RES (13)..(13): E or absent
  MOD_RES (14)..(14): Q or absent
  MOD_RES (15)..(15): A, K, or 2-amino isobutyric acid (Aib), or absent
  MOD_RES (16)..(16): K, modified Lys (K*), A, S, Q, H, T, or absent
  MOD_RES (17)..(17): T, L, D, Y, A, Aib, absent, or a modified/substituted alanine
  MOD_RES (18)..(18): F, S, T, H, N, D, E, A, Aib, or absent
  MOD_RES (19)..(19): L, E, A, or absent
  MOD_RES (20)..(20): D, E, A, cysteic acid (Cya), or absent
  MOD_RES (21)..(21): K, Q, W, A, absent, or a modified/substituted alanine
  MOD_RES (22)..(22): F, K, A, Aib, or absent
  MOD_RES (23)..(23): N, K, H, D, A, or absent
  SITE (23)..(43): When any of positions 23-33 are absent, all residues subsequent to the absent residue are also absent
  MOD_RES (24)..(24): H, E, V, A, Aib, 4-aminopiperidine-4-carboxylic acid (Pip), 4-fluorophenylalanine (Ff), or absent
  MOD_RES (25)..(25): E, A, or absent
  MOD_RES (26)..(26): A, E, Aib, Pip, or absent
  MOD_RES (27)..(27): E, A, or absent
  MOD_RES (28)..(28): D, A, or absent
  MOD_RES (29)..(29): L, K, A, Aib, ornithine (Orn), Pip or absent
  MOD_RES (30)..(30): F, K, A, Aib, or absent
  MOD_RES (31)..(31): Y, A, absent, or a modified/substituted alanine
  MOD_RES (32)..(32): Q, K, or absent
  MOD_RES (33)..(33): S, Aib or absent
  MOD_RES (34)..(43): This region may encompass one of the following sequences “S” or “SLAS” or “SGGLGKGDFR” or “SpLGKGDFR” or “SkLGKGDFR” or “SGLGKGDFR” or “SGLGKGCyaFR” or absent
  See specification as filed for detailed description of substitutions and preferred embodiments
XXXXXXXXXXXXXXXXXXXXXXXXXXXXXXXXXXXXXXXXXXX

SEQ ID NO: 135 - protein, 9 aa, Artificial Sequence
  Description of Artificial Sequence Synthetic peptide
AQMYPLQEG

SEQ ID NO: 136 - protein, 10 aa, Artificial Sequence
  Description of Artificial Sequence Synthetic peptide
AQMYPLQEGG

SEQ ID NO: 137 - protein, 4 aa, Artificial Sequence
  Description of Artificial Sequence Synthetic peptide
SLAS

SEQ ID NO: 138 - protein, 10 aa, Artificial Sequence
  Description of Artificial Sequence Synthetic peptide
SGGLGKGDFR

SEQ ID NO: 139 - protein, 9 aa, Artificial Sequence
  Description of Artificial Sequence Synthetic peptide
  MOD_RES (2)..(2): D-amino acid
SPLGKGDFR

SEQ ID NO: 140 - protein, 9 aa, Artificial Sequence
  Description of Artificial Sequence Synthetic peptide
  MOD_RES (2)..(2): D-amino acid
SKLGKGDFR

SEQ ID NO: 141 - protein, 9 aa, Artificial Sequence
  Description of Artificial Sequence Synthetic peptide
SGLGKGDFR

SEQ ID NO: 142 - protein, 9 aa, Artificial Sequence
  Description of Artificial Sequence Synthetic peptide
  MOD_RES (7)..(7): Cya
SGLGKGXFR

SEQ ID NO: 143 - protein, 43 aa, Artificial Sequence; identical to SEQ ID NO: 134
  Description of Artificial Sequence Synthetic polypeptide
  MOD_RES (1)..(10): This region may encompass one of the following sequences “QST” or “AQMYPLQEG” or “AQMYPLQEGG” or absent
  SITE (1)..(22): When any of positions 11-22 are absent, all residues prior to the absent residue are also absent
  SITE (1)..(43): Wherein at least 12 consecutive residues are not absent
  SITE (1)..(43): The residue at position 15 may be stapled with the residue at either position 18 or 22
  SITE (1)..(43): The residue at position 18 may be stapled with the residue at either position 15, 22, or 26
  SITE (1)..(43): The residue at position 22 may be stapled with the residue at either position 15, 18, or 26
  SITE (1)..(43): The residue at position 26 may be stapled with the residue at either position 18, 22, or 30
  SITE (1)..(43): The residue at position 30 may be stapled with the residue at position 26
  MOD_RES (11)..(11): I or absent
  MOD_RES (12)..(12): E or absent
  MOD_RES (13)..(13): E or absent
  MOD_RES (14)..(14): Q or absent
  MOD_RES (15)..(15): A, K, or 2-amino isobutyric acid (Aib), or absent
  MOD_RES (16)..(16): K, modified Lys (K*), A, S, Q, H, T, or absent
  MOD_RES (17)..(17): T, L, D, Y, A, Aib, or absent
  MOD_RES (18)..(18): F, S, T, H, N, D, E, A, Aib, or absent
  MOD_RES (19)..(19): L, E, A, or absent
  MOD_RES (20)..(20): D, E, A, cysteic acid (Cya), or absent
  MOD_RES (21)..(21): K, Q, W, A, absent, or a modified/substituted alanine
  MOD_RES (22)..(22): F, K, A, Aib, or absent
  MOD_RES (23)..(23): N, K, H, D, A, or absent
  SITE (23)..(43): When any of positions 23-33 are absent, all residues subsequent to the absent residue are also absent
  MOD_RES (24)..(24): H, E, V, A, Aib, 4-aminopiperidine-4-carboxylic acid (Pip), 4-fluorophenylalanine (Ff), or absent
  MOD_RES (25)..(25): E, A, or absent
  MOD_RES (26)..(26): A, E, Aib, Pip, or absent
  MOD_RES (27)..(27): E, A, or absent
  MOD_RES (28)..(28): D, A, or absent
  MOD_RES (29)..(29): L, K, A, Aib, ornithine (Orn), Pip or absent
  MOD_RES (30)..(30): F, K, A, Aib, or absent
  MOD_RES (31)..(31): Y, A, or absent
  MOD_RES (32)..(32): Q, K, or absent
  MOD_RES (33)..(33): S, Aib or absent
  MOD_RES (34)..(43): This region may encompass one of the following sequences “S” or “SLAS” or “SGGLGKGDFR” or “SpLGKGDFR” or “SkLGKGDFR” or “SGLGKGDFR” or “SGLGKGCyaFR” or absent
  See specification as filed for detailed description of substitutions and preferred embodiments